(19) United States
(12) Patent Application Publication
(10) Pub. No.: US 2013/0096136 A1
(43) Pub. Date: Apr. 18, 2013

(54) Inhibitors of Protein Tyrosine Kinase Activity

(71) Applicant: METHYLGENE INC., Montreal (CA)

(72) Inventors: Seiji Hata, Osaka (JP); Yohei Yuki, Osaka (JP); Stephen William Claridge, Montreal (CA); Stéphane Raeppel, St. Lazare (CA); Arkadii Vaisburg, Kirkland (CA)

(73) Assignee: METHYLGENE INC., Montreal (CA)

(21) Appl. No.: 13/629,019

(22) Filed: Sep. 27, 2012

Related U.S. Application Data
(60) Provisional application No. 61/541,330, filed on Sep. 30, 2011.

(51) Int. Cl. C07D 495/04

(52) U.S. Cl. CPC C07D 495/04
USPC 514/253.04; 546/114; 514/301; 544/362

(57) ABSTRACT

The present invention provides new compounds and methods for treating a disease responsive to inhibition of kinase activity, for example a disease responsive to inhibition of protein tyrosine kinase activity, for example a disease responsive to inhibition of protein tyrosine kinase activity of growth factor receptors, for example a disease responsive to inhibition of receptor type tyrosine kinase signaling, or for example, a disease responsive to inhibition of VEGF receptor signaling.

### FIELD OF THE INVENTION

This invention relates to compounds that inhibit protein tyrosine kinase activity. In particular the invention relates to compounds that inhibit the protein tyrosine kinase activity of growth factor receptors, resulting in the inhibition of receptor signaling, for example, the inhibition of VEGF receptor signaling. More particularly, the invention relates to compounds, compositions and methods for the inhibition of VEGF receptor signaling.

### SUMMARY OF THE RELATED ART

Tyrosine kinases may be classified as growth factor receptor (e.g. EGFR, PDGFR, FGFR and erbB2) or non-receptor (e.g. c-src and bcr-abl) kinases. The receptor type tyrosine kinases make up about 20 different subfamilies. The non-receptor type tyrosine kinases make up numerous subfamilies. These tyrosine kinases have diverse biological activity. Receptor tyrosine kinases are large enzymes that span the cell membrane and possess an extracellular binding domain for growth factors, a transmembrane domain, and an intracellular portion that functions as a kinase to phosphorylate a specific tyrosine residue in proteins and hence to influence cell proliferation. Aberrant or inappropriate protein kinase activity can contribute to the rise of disease states associated with such aberrant kinase activity.

Angiogenesis is an important component of certain normal physiological processes such as embryogenesis and wound healing, but aberrant angiogenesis contributes to some pathological disorders and in particular to tumor growth. VEGF-A (vascular endothelial growth factor A) is a key factor promoting neovascularization (angiogenesis) of tumors. VEGF induces endothelial cell proliferation and migration by signaling through two high affinity receptors, the fms-like tyrosine kinase receptor, Flt-1, and the kinase insert domain-containing receptor, KDR. These signaling responses are critically dependent upon receptor dimerization and activation of intrinsic receptor tyrosine kinase (RTK) activity. The binding of VEGF as a disulfide-linked homodimer stimulates receptor dimerization and activation of the RTK domain. The kinase autophosphorylates cytoplasmic receptor tyrosine residues, which then serve as binding sites for molecules involved in the propagation of a signaling cascade. Although multiple pathways are likely to be elucidated for both receptors, KDR signaling is most extensively studied, with a mitogenic response suggested to involve ERK-1 and ERK-2 mitogen-activated protein kinases.

Disruption of VEGF receptor signaling is a highly attractive therapeutic target in cancer, as angiogenesis is a prerequisite for all solid tumor growth, and that the mature endothelium remains relatively quiescent (with the exception of the female reproductive system and wound healing). A number of experimental approaches to inhibiting VEGF signaling have been examined, including use of neutralizing antibodies, receptor antagonists, soluble receptors, antisense constructs and dominant-negative strategies.

Tyrosine kinases also contribute to the pathology of ophthalmic diseases, disorders and conditions, such as age-related macular degeneration (AMD) and diabetic retinopathy (DR). Blindness from such diseases has been linked to anomalies in retinal neovascularization. The formation of new blood vessels is regulated by growth factors such as VEGF and HGF that activate receptor tyrosine kinases resulting in the initiation of signaling pathways leading to plasma leakage into the macula, causing vision loss. Kinases are thus attractive targets for the treatment of eye diseases involving neovascularization.

Thus, there is a need to develop a strategy for controlling neovascularization of the eye and to develop medicines for the treatment of ocular diseases.

Here we describe small molecules that are potent inhibitors of protein tyrosine kinase activity.

### BRIEF SUMMARY OF THE INVENTION

The present invention provides new compounds and methods for treating a disease responsive to inhibition of kinase activity, for example a disease responsive to inhibition of protein tyrosine kinase activity, for example a disease responsive to inhibition of protein tyrosine kinase activity of growth factor receptors, for example a disease responsive to inhibition of receptor type tyrosine kinase signaling, or for example, a disease responsive to inhibition of VEGF receptor signaling. In some embodiments the disease is a cell proliferative disease. In other embodiments, the disease is an ophthalmic disease. The compounds of the invention are inhibitors of kinase activity, such as protein tyrosine kinase activity, for example protein tyrosine kinase activity of growth factor receptors, or for example receptor type tyrosine kinase signaling.

In a first aspect, the invention provides compounds of Formula (I) that are useful as kinase inhibitors:

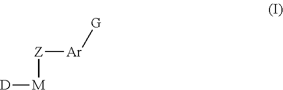

and N-oxides, hydrates, solvates, tautomers, pharmaceutically acceptable salts, prodrugs and complexes thereof, and racemic and scalemic mixtures, diastereomers and enantiomers thereof, wherein D, M, Z, Ar and G are as defined herein. Because compounds of the present invention are useful as kinase inhibitors they are, therefore, useful research tools for the study of the role of kinases in both normal and disease states. In some embodiments, the invention provides compounds that are useful as inhibitors of VEGF receptor signaling and, therefore, are useful research tools for the study of the role of VEGF in both normal and disease states.

In a second aspect, the invention provides compositions comprising a compound according to the present invention and a pharmaceutically acceptable carrier, excipient or diluent. For example, the invention provides compositions comprising a compound that is an inhibitor of VEGF receptor signaling, or a pharmaceutically acceptable salt thereof, and a pharmaceutically acceptable carrier, excipient, or diluent.

In a third aspect, the invention provides a method of inhibiting kinase activity, for example protein tyrosine kinase, for example tyrosine kinase activity of a growth factor receptor, the method comprising contacting the kinase with a compound according to the present invention, or with a composition according to the present invention. In some embodiments of this aspect, the invention provides a method of inhibiting receptor type tyrosine kinase signaling, for example inhibiting VEGF receptor signaling. Inhibition can be in a cell or a multicellular organism. If in a cell, the method according to this aspect of the invention comprises contacting the cell with a compound according to the present invention, or with a composition according to the present invention. If in a multicellular organism, the method according to this aspect of the invention comprises administering to the organism a compound according to the present invention, or a composition according to the present invention. In some embodiments the organism is a mammal, for example a primate, for example a human.

In a fourth aspect, the invention provides a method of inhibiting angiogenesis, the method comprising administering to a patient in need thereof a therapeutically effective amount of a compound according to the present invention, or a therapeutically effective amount of a composition according to the present invention. In some embodiments of this aspect, the angiogenesis to be inhibited is involved in tumor growth. In some other embodiments the angiogenesis to be inhibited is retinal angiogenesis. In some embodiments of this aspect, the patient is a mammal, for example a primate, for example a human.

In a fifth aspect, the invention provides a method of treating a disease responsive to inhibition of kinase activity, for example a disease responsive to inhibition of protein tyrosine kinase activity, for example a disease responsive to inhibition of protein tyrosine kinase activity of growth factor receptors. In some embodiments of this aspect, the invention provides a method of treating a disease responsive to inhibition of receptor type tyrosine kinase signaling, for example a disease responsive to inhibition of VEGF receptor signaling, the method comprising administering to an organism in need thereof a therapeutically effective amount of a compound according to the present invention, or a composition according to the present invention. In some embodiments of this aspect, the organism is a mammal, for example a primate, for example a human.

In a sixth aspect, the invention provides a method of treating a cell proliferative disease, the method comprising administering to a patient in need thereof a therapeutically effective amount of a compound according to the present invention, or a therapeutically effective amount of a composition according to the present invention. In some embodiments of this aspect, the cell proliferative disease is cancer. In some embodiments, the patient is a mammal, for example a primate, for example a human.

In a seventh aspect, the invention provides a method of treating an ophthalmic disease, disorder or condition, the method comprising administering to a patient in need thereof a therapeutically effective amount of a compound according to the present invention, or a therapeutically effective amount of a composition according to the present invention. In some embodiments of this aspect, the disease is caused by choroidal angiogenesis. In some embodiments of this aspect, the patient is a mammal, for example a primate, for example a human.

In an eighth aspect, the invention provides for the use of a compound according to the present invention for or in the manufacture of a medicament to inhibit kinase activity, for example to inhibit protein tyrosine kinase activity, for example to inhibit protein tyrosine kinase activity of growth factor receptors. In some embodiments of this aspect, the invention provides for the use of a compound according to the present invention for or in the manufacture of a medicament to inhibit receptor type tyrosine kinase signaling, for example to inhibit VEGF receptor signaling. In some embodiments of this aspect, the invention provides for the use of a compound according to the present invention for or in the manufacture of a medicament to treat a disease responsive to inhibition of kinase activity. In some embodiments of this aspect, the disease is responsive to inhibition of protein tyrosine kinase activity, for example inhibition of protein tyrosine kinase activity of growth factor receptors. In some embodiments of this aspect, the disease is responsive to inhibition of receptor type tyrosine kinase signaling, for example VEGF receptor signaling. In some embodiments of this aspect, the disease is a cell proliferative disease, for example cancer. In some embodiments of this aspect, the disease is an ophthalmic disease, disorder or condition. In some embodiments of this aspect, the ophthalmic disease, disorder or condition is caused by choroidal angiogenesis. In some embodiments of this aspect, the disease is age-related macular degeneration, diabetic retinopathy or retinal edema.

In a ninth aspect, the invention provides for the use of a compound according to the present invention, or a composition thereof, to inhibit kinase activity, for example to inhibit receptor type tyrosine kinase activity, for example to inhibit protein tyrosine kinase activity of growth factor receptors. In some embodiments of this aspect, the invention provides for the use of a compound according to the present invention, or a composition thereof, to inhibit receptor type tyrosine kinase signaling, for example to inhibit VEGF receptor signaling.

In a tenth aspect, the invention provides for the use of a compound according to the present invention, or a composition thereof, to treat a disease responsive to inhibition of kinase activity, for example a disease responsive to inhibition of protein tyrosine kinase activity, for example a disease responsive to inhibition or protein tyrosine kinase activity of growth factor receptors. In some embodiments of this aspect, the invention provides for the use of a compound according to the present invention, or a composition thereof, to treat a disease responsive to inhibition of receptor type tyrosine kinase signaling, for example a disease responsive to inhibition of VEGF receptor signaling. In some embodiments of this aspect, the disease is a cell proliferative disease, for example cancer. In some embodiments of this aspect, the disease is an ophthalmic disease, disorder or condition. In some embodiments of this aspect, the ophthalmic disease, disorder or condition is caused by choroidal angiogenesis.

The foregoing merely summarizes some aspects of the invention and is not intended to be limiting in nature. These aspects and other aspects and embodiments are described more fully below.

### DETAILED DESCRIPTION

The invention provides compounds, compositions and methods for inhibiting kinase activity, for example protein tyrosine kinase activity, for example receptor protein kinase activity, for example the VEGF receptor KDR. The invention also provides compounds, compositions and methods for inhibiting angiogenesis, treating a disease responsive to inhibition of kinase activity, treating cell proliferative diseases and conditions and treating ophthalmic diseases, disorders and conditions. The patent and scientific literature referred to herein reflects knowledge that is available to those with skill in the art. The issued patents, published patent applications, and references that are cited herein are hereby incorporated by reference to the same extent as if each was specifically and individually indicated to be incorporated by reference. In the case of inconsistencies, the present disclosure will prevail.

For purposes of the present invention, the following abbreviations will be used (unless expressly stated otherwise)

| | |
|---|---|
| Ac | acetyl |
| AcOEt | ethyl acetate |
| AcOH | acetic acid |
| aq | aqueous |
| bd | broad doublet (NMR) |
| Bn | benzyl |
| Boc | tert-butoxycarbonyl |
| br s | broad singlet (NMR) |
| CV | column volume |
| d | doublet (NMR) |
| dd | doublet of doublets (NMR) |
| DCC | dicyclohexyl carbodiimide |
| DCM | dichloromethane |
| DEAD | diethyl diazenedicarboxylate |
| DIPEA | diisopropyl ethylamine |
| DMAP | N, N-dimethylamino pyridine |
| DMF | N, N-dimethylformamide |
| DMSO | dimethylsulfoxide |
| DMSO-d₆ | dimethylsulfoxide-d₆ |
| EDC | 1-(3-dimethylaminopropyl)- |
| | 3-ethyl-carbodiimide |
| Et | ethyl |
| EDCI | 1-(3-dimethylaminopropyl)- |
| | 3-ethyl-carbodiimide |
| Et₃N | triethylamine |
| EtOH | ethanol |
| EtOAc | ethyl acetate |
| Et₂O | diethyl ether |
| equiv | equivalent |
| g | gram (grams) |
| h | hour (hours) |
| HOBT | 1-hydroxybenzotriazole |
| m | multiplet (NMR) |
| mL | milliliter |
| μL | microliter |
| Me | methyl |
| MeOH | methanol |
| MeOH-d₄ | methanol-d₄ |
| mg | milligram (milligrams) |
| min | minute (minutes) |
| MS | mass-spectroscopy |
| m/z | mass-to-charge ratio |
| NMP | N-methyl-2-pyrrolidone |
| NMR | nuclear magnetic resonance |
| | spectroscopy |
| PEG | polyethylene glycol |
| Ph | phenyl |
| Ppm | parts per million(NMR) |
| rt | room temperature |
| s | singlet (NMR) |
| t | triplet (NMR) |
| TFA | trifluoroacetic acid |
| THF | tetrahydrofuran |

For purposes of the present invention, the following definitions will be used (unless expressly stated otherwise):

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” Similarly, in circumstances in which a divalent moiety is required and is stated as being “aryl,” those skilled in the art will understand that the term “aryl” refers to the corresponding divalent moiety, arylene. All atoms are understood to have their normal number of valences for bond formation (i.e., 4 for carbon, 3 for nitrogen, 2 for oxygen, and 2, 4, or 6 for sulfur, depending on the oxidation state of the S). On occasion a moiety may be defined, for example, as (A)ₐ-B-, wherein a is 0 or 1. In such instances, when a is 0 the moiety is B- and when a is 1 the moiety is A-B-.

For simplicity, reference to a “Cₙ-Cₘ” heterocyclyl or “Cₙ-Cₘ” heteroaryl means a heterocyclyl or heteroaryl having from “n” to “m” annular atoms, where “n” and “m” are integers. Thus, for example, a C₅-C₆heterocyclyl is a 5- or 6-membered ring having at least one heteroatom, and includes pyrrolidinyl (C₅) and piperazinyl and piperidinyl (C₆); C₆heteroaryl includes, for example, pyridyl and pyrimidyl.

The term “hydrocarbyl” refers to a straight, branched, or cyclic alkyl, alkenyl, or alkynyl, each as defined herein. A “C₀” hydrocarbyl is used to refer to a covalent bond. Thus, “C₀-C₃ hydrocarbyl” includes a covalent bond, methyl, ethyl, ethenyl, ethynyl, propyl, propenyl, propynyl, and cyclopropyl.

The term “alkyl” is intended to mean a straight chain or branched aliphatic group having from 1 to 12 carbon atoms, alternatively 1-8 carbon atoms, and alternatively 1-6 carbon atoms. In some embodiments, the alkyl group has 1-4 carbon atoms. In some embodiments, the alkyl groups have from 2 to 12 carbon atoms, alternatively 2-8 carbon atoms and alternatively 2-6 carbon atoms. In some embodiments, the alkyl group has 2-4 carbon atoms. Examples of alkyl groups include, without limitation, methyl, ethyl, propyl, isopropyl, butyl, isobutyl, sec-butyl, tert-butyl, pentyl, hexyl and the like. A “C₀” alkyl (as in “C₀-C₃alkyl”) is a covalent bond.

The term “alkenyl” is intended to mean an unsaturated straight chain or branched aliphatic group with one or more carbon-carbon double bonds, having from 2 to 12 carbon atoms, alternatively 2-8 carbon atoms, and alternatively 2-6 carbon atoms. In some embodiments, the alkenyl group has 2-4 carbon atoms. Examples alkenyl groups include, without limitation, ethenyl, propenyl, butenyl, pentenyl, and hexenyl.

The term “alkynyl” is intended to mean an unsaturated straight chain or branched aliphatic group with one or more carbon-carbon triple bonds, having from 2 to 12 carbon atoms, alternatively 2-8 carbon atoms, and alternatively 2-6 carbon atoms. In some embodiments, the alkynyl group has 2-4 carbon atoms. Examples of alkynyl groups include, without limitation, ethynyl, propynyl, butynyl, pentynyl, and hexynyl.

The terms “alkylene,” “alkenylene,” or “alkynylene” as used herein are intended to mean an alkyl, alkenyl, or alkynyl group, respectively, as defined hereinabove, that is positioned between and serves to connect two other chemical groups. Examples of alkylene groups include, without limitation, methylene, ethylene, propylene, and butylene. Examples of alkenylene groups include, without limitation, ethenylene, propenylene, and butenylene. Examples of alkynylene groups include, without limitation, ethynylene, propynylene, and butynylene.

The term “carbocycle” as employed herein is intended to mean a cycloalkyl or aryl moiety.

The term “cycloalkyl” is intended to mean a saturated, partially unsaturated or unsaturated mono-, bi-, tri- or poly-cyclic hydrocarbon group having about 3 to 15 carbons, alternatively having 3 to 12 carbons, alternatively 3 to 8 carbons, alternatively 3 to 6 carbons, and alternatively 5 or 6 carbons. In some embodiments, the cycloalkyl group is fused to an aryl, heteroaryl or heterocyclic group. Examples of cycloalkyl groups include, without limitation, cyclopenten-2-enone, cyclopenten-2-enol, cyclohex-2-enone, cyclohex-2-enol, cyclopropyl, cyclobutyl, cyclobutenyl, cyclopentyl, cyclopentenyl, cyclohexyl, cyclohexenyl, cycloheptyl, cyclooctyl, etc.

The term “heteroalkyl” is intended to mean a saturated, partially unsaturated or unsaturated, straight chain or branched aliphatic group, wherein one or more carbon atoms in the group are independently replaced by a heteroatom selected from the group consisting of O, S, and N.

The term “aryl” is intended to mean a mono-, bi-, tri- or polycyclic aromatic moiety, comprising one to three aromatic rings. In some embodiments the aryl is a C₆-C₁₄aromatic moiety, alternatively the aryl group is a C₆-C₁₀aryl group, alternatively a C₆ aryl group. Examples of aryl groups include, without limitation, phenyl, naphthyl, anthracenyl, and fluorenyl.

The terms “aralkyl” or “arylalkyl” are intended to mean a group comprising an aryl group covalently linked to an alkyl group. If an aralkyl group is described as “optionally substituted”, it is intended that either or both of the aryl and alkyl moieties may independently be optionally substituted or unsubstituted. In some embodiments, the aralkyl group is (C₁-C₆)alk(C₆-C₁₀)aryl, including, without limitation, benzyl, phenethyl, and naphthylmethyl. For simplicity, when written as “arylalkyl” this term, and terms related thereto, is intended to indicate the order of groups in a compound as “aryl-alkyl”. Similarly, “alkyl-aryl” is intended to indicate the order of the groups in a compound as “alkyl-aryl”.

The terms “heterocyclyl”, “heterocyclic” or “heterocycle” are intended to mean a group which is a mono-, bi-, or polycyclic structure having from about 3 to about 14 atoms, alternatively 3 to 8 atoms, alternatively 4 to 7 atoms, alternatively 5 or 6 atoms wherein one or more atoms, for example 1 or 2 atoms, are independently selected from the group consisting of N, O, and S, the remaining ring-constituting atoms being carbon atoms. The ring structure may be saturated, unsaturated or partially unsaturated. In some embodiments, the heterocyclic group is non-aromatic, in which case the group is also known as a heterocycloalkyl. In some embodiments the heterocyclyl is a spiro-heterocyclyl, such as 2,7-diazaspiro[4.4]nonane, 2,8-diazaspiro[5.5]undecane, 2,8-diazaspiro[4.5]decane, 2,7-diazaspiro[3.5]nonane, 2,6-diazaspiro[3.4]octane, 2-oxa-7-azaspiro[4.4]nonane, 2-oxa-8-azaspiro[5.5]undecane, 8-oxa-2-azaspiro[4.5]decane, 7-oxa-2-azaspiro[3.5]nonane, 6-oxa-2-azaspiro[3.4]octane, 1-oxa-7-azaspiro[4.4]nonane, 2-oxa-8-azaspiro[5.5]undecane, 2-oxa-8-azaspiro[4.5]decane, 2-oxa-7-azaspiro[3.5]nonane and 2-oxa-6-azaspiro[3.4]octane. In a bicyclic or polycyclic structure, one or more rings may be aromatic; for example, one ring of a bicyclic heterocycle or one or two rings of a tricyclic heterocycle may be aromatic, as in indan and 9,10-dihydro anthracene. Examples of heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, morpholino, thienyl, pyridyl, 1,2,3-triazolyl, imidazolyl, isoxazolyl, pyrazolyl, piperazino, piperidyl, piperidino, morpholinyl, homopiperazinyl, homopiperazino, thiomorpholinyl, thiomorpholino, tetrahydropyrrolyl, and azepanyl. In some embodiments, the heterocyclic group is fused to an aryl, heteroaryl, or cycloalkyl group. Examples of such fused heterocycles include, without limitation, tetrahydroquinoline and dihydrobenzofuran. Specifically excluded from the scope of this term are compounds where an annular O or S atom is adjacent to another O or S atom.

In some embodiments, the heterocyclic group is a heteroaryl group. As used herein, the term “heteroaryl” is intended to mean a mono-, bi-, tri- or polycyclic group having 5 to 14 ring atoms, alternatively 5, 6, 9, or 10 ring atoms; having for example 6, 10, or 14 pi electrons shared in a cyclic array; and having, in addition to carbon atoms, between one or more heteroatoms independently selected from the group consisting of N, O, and S. For example, a heteroaryl group includes, without limitation, pyrimidinyl, pyridinyl, benzimidazolyl, thienyl, benzothiazolyl, benzofuranyl and indolinyl. Other examples of heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, and isoxazolyl.

The terms “arylene,” “heteroarylene,” or “heterocyclylene” are intended to mean an aryl, heteroaryl, or heterocyclyl group, respectively, as defined hereinabove, that is positioned between and serves to connect two other chemical groups.

Examples of heterocyclyls and heteroaryls include, but are not limited to, azepinyl, azetidinyl, acridinyl, azocinyl, benzidolyl, benzimidazolyl, benzofuranyl, benzofurazanyl, benzofuryl, benzothiofuranyl, benzothiophenyl, benzoxazolyl, benzothiazolyl, benzothienyl, benztriazolyl, benztetrazolyl, benzisoxazolyl, benzisothiazolyl, benzimidazolinyl, benzoxazolyl, benzoxadiazolyl, benzopyranyl, carbazolyl, 4aH-carbazolyl, carbolinyl, chromanyl, chromenyl, cinnolinyl, coumarinyl, decahydroquinolinyl, 1,3-dioxolane, 2H,6H-1,5,2-dithiazinyl, dihydrofuro[2,3-b]tetrahydrofuran, dihydroisoindolyl, dihydroquinazolinyl (such as 3,4-dihydro-4-oxo-quinazolinyl), furanyl, furopyridinyl (such as furo[2,3-c]pyridinyl, furo[3,2-b]pyridinyl or furo[2,3-b]pyridinyl), furyl, furazanyl, hexahydrodiazepinyl, imidazolidinyl, imidazolinyl, imidazolyl, indazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolidinyl, isothiazolyl, isoxazolinyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, oxetanyl, 2-oxoazepinyl, 2-oxopiperazinyl, 2-oxopiperidinyl, 2-oxopyrrolodinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolopyridyl, 2H-pyrrolyl, pyrrolyl, quinazolinyl, quinolinyl, 4H-quinolizinyl, quinoxalinyl, quinuclidinyl, tetrahydro-1,1-dioxothienyl, tetrahydrofuranyl, tetrahydrofuryl, tetrahydroisoquinolinyl, tetrahydroquinolinyl, tetrahydropyranyl, tetrazolyl, thiazolidinyl, 6H-1,2,5-thiadiazinyl, thiadiazolyl (e.g., 1,2,3-thiadiazolyl, 1,2,4-thiadiazolyl, 1,2,5-thiadiazolyl, 1,3,4-thiadiazolyl), thiamorpholinyl, thiamorpholinyl sulfoxide, thiamorpholuiyl sulfone, thianthrenyl, thiazolyl, thienyl, thienothiazolyl, thienooxazolyl, thienoimidazolyl, thiophenyl, triazinyl, triazinylazepinyl, triazolyl (e.g., 1,2,3-triazolyl, 1,2,4-triazolyl, 1,2,5-triazolyl, 1,3,4-triazolyl), and xanthenyl.

The term “azolyl” as employed herein is intended to mean a five-membered saturated or unsaturated heterocyclic group containing two or more hetero-atoms, as ring atoms, selected from the group consisting of nitrogen, sulfur and oxygen, wherein at least one of the hetero-atoms is a nitrogen atom. Examples of azolyl groups include, but are not limited to, optionally substituted imidazolyl, oxazolyl, thiazolyl, pyrazolyl, isoxazolyl, isothiazolyl, 1,3,4-thiadiazolyl, 1,2,4-thiadiazolyl, 1,2,4-oxadiazolyl, and 1,3,4-oxadiazolyl.

As employed herein, and unless stated otherwise, when a moiety (e.g., alkyl, heteroalkyl, cycloalkyl, aryl, heteroaryl, heterocyclyl, etc.) is described as “optionally substituted” it is meant that the group optionally has from one to four, alternatively from one to three, alternatively one or two, independently selected non-hydrogen substituents. Suitable substituents include, without limitation, halogen, hydroxy, oxo (e.g., an annular —CH— substituted with oxo is —C(O)—) nitro, halohydrocarbyl, hydrocarbyl, alkyl, cycloalkyl, heterocyclyl, aryl, heteroaryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups.

Examples of substituents, which are themselves not further substituted (unless expressly stated otherwise) are:
(a) halogen, cyano, oxo, carboxy, formyl, nitro, amino, amidino, guanidino,
(b) C₁-C₅alkyl or alkenyl or arylalkylimino, carbamoyl, azido, carboxamido, mercapto, hydroxy, hydroxyalkyl, alkylaryl, arylalkyl, C₁-C₈alkyl, C₂-C₈alkenyl, C₁-C₈alkoxy, C₁-C₈alkylamino, C₁-C₈alkoxycarbonyl, aryloxycarbonyl, C₂-C₈acyl, C₂-C₈acylamino, C₁-C₈alkylthio, arylalkylthio, arylthio, C₁-C₈alkylsulfinyl, arylalkylsulfinyl, arylsulfinyl, C₁-C₈alkylsulfonyl, arylalkylsulfonyl, arylsulfonyl, C₀-C₆N-alkyl carbamoyl, C₂-C₁₅N,N-dialkylcarbamoyl, C₃-C₇ cycloalkyl, aroyl, aryloxy, arylalkyl ether, aryl, aryl fused to a cycloalkyl or heterocycle or another aryl ring, C₃-C₇heterocycle, C₅-C₁₅heteroaryl or any of these rings fused or spiro-fused to a cycloalkyl, heterocyclyl, or aryl, wherein each of the foregoing is further optionally substituted with one more moieties listed in (a), above; and
(c) —(CR³²R³³)ₛ—NR³⁰R³¹,
   wherein s is from 0 (in which case the nitrogen is directly bonded to the moiety that is substituted) to 6,
   R³² and R³³ are each independently hydrogen, halogen, hydroxyl or C₁-C₄alkyl, and
   R³⁰ and R³¹ are each independently hydrogen, cyano, oxo, hydroxyl, C₁-C₈alkyl, C₁-C₈heteroalkyl, C₂-C₈alkenyl, carboxamido, C₁-C₃alkyl-carboxamido, carboxamido-C₁-C₃alkyl, amidino, C₂-C₈hydroxyalkyl, C₁-C₃alkylaryl, aryl-C₁-C₃alkyl, C₁-C₃alkylheteroaryl, hetero aryl-C₁-C₃alkyl, C₁-C₃alkylheterocyclyl, heterocyclyl-C₁-C₃alkyl C₁-C₃alkylcycloalkyl, cyclo alkyl-C₁-C₃alkyl, C₂-C₈alkoxy, C₂-C₈alkoxy-C₁-C₄alkyl, C₁-C₈alkoxycarbonyl, aryloxycarbonyl, aryl-C₁-C₃alkoxycarbonyl, heteroaryloxycarbonyl, hetero aryl-C₁-C₃alkoxycarbonyl, C₁-C₈acyl, C₀-C₈alkyl-carbonyl, aryl-C₀-C₈alkyl-carbonyl, heteroaryl-C₀-C₈alkyl-carbonyl, cycloalkyl-C₀-C₈alkyl-carbonyl, C₀-C₈alkyl-NH-carbonyl, aryl-C₀-C₈alkyl-NH-carbonyl, heteroaryl-C₀-C₈alkyl-NH-carbonyl, cycloalkyl-C₀-C₈alkyl-NH-carbonyl, C₀-C₈alkyl-O-carbonyl, aryl-C₀-C₈alkyl-O-carbonyl, heteroaryl-C₀-C₈alkyl-O-carbonyl, cycloalkyl-C₀-C₈alkyl-O-Carbonyl, C₁-C₈alkylsulfonyl, arylalkylsulfonyl, arylsulfonyl, heteroarylalkylsulfonyl, heteroarylsulfonyl, C₁-C₈alkyl-NH-sulfonyl, arylalkyl-NH-sulfonyl, aryl-NH-sulfonyl, heteroarylalkyl-NH-sulfonyl, heteroaryl-NH-sulfonyl aroyl, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, or protecting group, wherein each of the foregoing is further optionally substituted with one more moieties listed in (a), above; or
   R³⁰ and R³¹ taken together with the N to which they are attached form a heterocyclyl or heteroaryl, each of which is optionally substituted with from 1 to 3 substituents selected from the group consisting of (a) above, a protecting group, and (X³⁰—Y³¹—), wherein said heterocyclyl may also be bridged (forming a bicyclic moiety with a methylene, ethylene or propylene bridge); wherein
   X³⁰ is selected from the group consisting of C₁-C₈alkyl, C₂-C₈alkenyl-, C₂-C₈alkynyl-, —C₀-C₃alkyl-C₂-C₈alkenyl-C₀-C₃alkyl, C₀-C₃alkyl-C₂-C₈alkynyl-C₀-C₃alkyl, C₀-C₃alkyl-O—C₀-C₃alkyl-, HO—C₀-C₃alkyl-, C₀-C₄alkyl-N(R³⁰)—C₀-C₃alkyl-, N(R³⁰)(R³¹)—C₀-C₃alkyl-, N(R³⁰)(R³¹)—C₀-C₃alkenyl-, N(R³⁰)(R³¹)—C₀-C₃alkynyl-, (N(R³⁰)(R³¹))₂—C═N—, C₀-C₃alkyl-S(O)₀₋₂—C₀-C₃alkyl-, CF₃—C₀-C₃alkyl-, C₁-C₈heteroalkyl, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, N(R³⁰)(R³¹)-heterocyclyl-C₁-C₃alkyl-, wherein the aryl, cycloalkyl, heteroaryl and heterocyclyl are optionally substituted with from 1 to 3 substituents from (a); and
   Y³¹ is selected from the group consisting of a direct bond, —O—, —N(R³⁰)—, —C(O)—, —O—C(O)—, —C(O)—O—, —N(R³⁰)—C(O)—, —C(O)—N(R³⁰)—, —N(R³⁰)—C(S)—, —C(S)—N(R³⁰)—, —N(R³⁰)—C(O)—N(R³¹)—, —N(R³⁰)—C(NR³⁰)—N(R³¹)—, —N(R³⁰)—C(NR³¹)—, —C(NR³¹)—N(R³⁰)—, —N(R³⁰)—C(S)—N(R³¹)—, —N(R³⁰)—C(O)—O—, —O—C(O)—N(R³¹)—, —N(R³⁰)—C(S)—O—, —O—C(S)—N(R³¹)—, —S(O)₀₋₂—, —SO₂N(R³¹)—, —N(R³¹)—SO₂— and —N(R³⁰)—SO₂N(R³¹)—.

A moiety that is substituted is one in which one or more (for example one to four, alternatively from one to three and alternatively one or two), hydrogens have been independently replaced with another chemical substituent. As a non-limiting example, substituted phenyls include 2-fluorophenyl, 3,4-dichlorophenyl, 3-chloro-4-fluoro-phenyl, 2-fluoro-3-propylphenyl. As another non-limiting example, substituted n-octyls include 2,4-dimethyl-5-ethyl-octyl and 3-cyclopentyl-octyl. Included within this definition are methylenes (—CH₂—) substituted with oxygen to form carbonyl (—CO—).

When there are two optional substituents bonded to adjacent atoms of a ring structure, such as for example a phenyl, thiophenyl, or pyridinyl, the substituents, together with the atoms to which they are bonded, optionally form a 5- or 6-membered cycloalkyl or heterocycle having 1, 2, or 3 annular heteroatoms.

In some embodiments, a hydrocarbyl, heteroalkyl, heterocyclic and/or aryl group is unsubstituted.

In some embodiments, a hydrocarbyl, heteroalkyl, heterocyclic and/or aryl group is substituted with from 1 to 3 independently selected substituents.

Examples of substituents on alkyl groups include, but are not limited to, hydroxyl, halogen (e.g., a single halogen substituent or multiple halogen substituents; in the latter case, groups such as CF₃ or an alkyl group bearing Cl₃), oxo, cyano, nitro, alkyl, cycloalkyl, alkenyl, cycloalkenyl, alkynyl, heterocycle, aryl, —OR^{a}, —SR^{a}, —S(═O)R^{e}, —S(═O)₂R^{e}, —P(═O)₂R^{e}, —S(═O)₂OR^{e}, —P(═O)₂OR^{e}, —NR^{b}R^{c}, —NR^{b}S(═O)₂R^{e}, —NR^{b}P(═O)₂R^{e}, —S(═O)₂NR^{b}R^{c}, —P(═O)₂NR^{b}R^{c}, —C(═O)OR^{e}, —C(═O)R^{a}, —C(═O)NR^{b}R^{c}, —OC(═O)R^{a}, —OC(═O)NR^{b}R^{c}, —NR^{b}C(═O)OR^{e}, —NR^{d}C(═O)NR^{b}R^{c}, —NR^{d}S(═O)₂NR^{b}R^{c}, —NR^{d}P(═O)₂NR^{b}R^{c}, —NR^{b}C(═O)R^{a} or —NR^{b}P(═O)₂R^{e}, wherein R^{a} is hydrogen, alkyl, cycloalkyl, alkenyl, cycloalkenyl, alkynyl, heterocycle or aryl; R^{b}, R^{c} and R^{d} are independently hydrogen, alkyl, cycloalkyl, heterocycle or aryl, or said R^{b} and R^{c} together with the N to which they are bonded optionally form a heterocycle; and R^{e} is alkyl, cycloalkyl, alkenyl, cycloalkenyl, alkynyl, heterocycle or aryl. In the aforementioned exemplary substituents, groups such as alkyl, cycloalkyl, alkenyl, alkynyl, cycloalkenyl, heterocycle and aryl can themselves be optionally substituted.

Examples of substituents on alkenyl and alkynyl groups include, but are not limited to, alkyl or substituted alkyl, as well as those groups recited as examples of alkyl substituents.

Examples of substituents on cycloalkyl groups include, but are not limited to, nitro, cyano, alkyl or substituted alkyl, as well as those groups recited above as examples of alkyl substituents. Other examples of substituents include, but are not limited to, spiro-attached or fused cyclic substituents, for example, spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

Examples of substituents on cycloalkenyl groups include, but are not limited to, nitro, cyano, alkyl or substituted alkyl, as well as those groups recited as examples of alkyl substituents. Other examples of substituents include, but are not limited to, spiro-attached or fused cyclic substituents, for examples spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

Examples of substituents on aryl groups include, but are not limited to, nitro, cycloalkyl or substituted cycloalkyl, cycloalkenyl or substituted cycloalkenyl, cyano, alkyl or substituted alkyl, as well as those groups recited above as examples of alkyl substituents. Other examples of substituents include, but are not limited to, fused cyclic groups, such as fused cycloalkyl, fused cycloalkenyl, fused heterocycle, or fused aryl, where the aforementioned cycloalkyl, cycloalkenyl, heterocycle and aryl substituents can themselves be optionally substituted. Still other examples of substituents on aryl groups (phenyl, as a non-limiting example) include, but are not limited to, haloalkyl and those groups recited as examples of alkyl substituents.

Examples of substituents on heterocyclic groups include, but are not limited to, cycloalkyl, substituted cycloalkyl, cycloalkenyl, substituted cycloalkenyl, nitro, oxo (i.e., ═O), cyano, alkyl, substituted alkyl, as well as those groups recited as examples of alkyl substituents. Other examples of substituents on heterocyclic groups include, but are not limited to, spiro-attached or fused cyclic substituents at any available point or points of attachment, for example spiro-attached cycloalkyl, spiro-attached cycloalkenyl, spiro-attached heterocycle (excluding heteroaryl), fused cycloalkyl, fused cycloalkenyl, fused heterocycle and fused aryl, where the aforementioned cycloalkyl, cycloalkenyl, heterocycle and aryl substituents can themselves be optionally substituted.

In some embodiments, a heterocyclic group is substituted on carbon, nitrogen and/or sulfur at one or more positions. Examples of substituents on nitrogen include, but are not limited to alkyl, aryl, aralkyl, alkylcarbonyl, alkylsulfonyl, arylcarbonyl, arylsulfonyl, alkoxycarbonyl, or aralkoxycarbonyl. Examples of substituents on sulfur include, but are not limited to, oxo and C₁₋₆alkyl. In some embodiments, nitrogen and sulfur heteroatoms may independently be optionally oxidized and nitrogen heteroatoms may independently be optionally quaternized.

In some embodiments, substituents on ring groups, such as aryl, heteroaryl, cycloalkyl and heterocyclyl, include halogen, alkoxy and/or alkyl.

In some embodiments, substituents on alkyl groups include halogen and/or hydroxy.

A “halohydrocarbyl” as employed herein is a hydrocarbyl moiety, in which from one to all hydrogens have been replaced with halogen.

The term “halogen” or “halo” as employed herein refers to chlorine, bromine, fluorine, or iodine. As herein employed, the term “acyl” refers to an alkylcarbonyl or arylcarbonyl substituent. The term “acylamino” refers to an amide group attached at the nitrogen atom (i.e., R—CO—NH—). The term “carbamoyl” refers to an amide group attached at the carbonyl carbon atom (i.e., NH₂—CO—). The nitrogen atom of an acylamino or carbamoyl substituent is additionally optionally substituted. The term “sulfonamido” refers to a sulfonamide substituent attached by either the sulfur or the nitrogen atom. The term “amino” is meant to include NH₂, alkylamino, dialkylamino (wherein each alkyl may be the same or different), arylamino, and cyclic amino groups. The term “ureido” as employed herein refers to a substituted or unsubstituted urea moiety.

The term “radical” as used herein means a chemical moiety comprising one or more unpaired electrons.

Where optional substituents are chosen from “one or more” groups it is to be understood that this definition includes all substituents being chosen from within one of the specified groups or from within the combination of all of the specified groups.

In addition, substituents on cyclic moieties (i.e., cycloalkyl, heterocyclyl, aryl, heteroaryl) include 5- to 6-membered mono- and 9- to 14-membered bi-cyclic moieties fused to the parent cyclic moiety to form a bi- or tri-cyclic fused ring system. Substituents on cyclic moieties also include 5- to 6-membered mono- and 9- to 14-membered bi-cyclic moieties attached to the parent cyclic moiety by a covalent bond to form a bi- or tri-cyclic bi-ring system. For example, an optionally substituted phenyl includes, but is not limited to, the following:

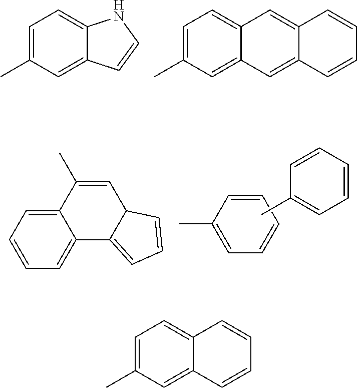

An “unsubstituted” moiety (e.g., unsubstituted cycloalkyl, unsubstituted heteroaryl, etc.) means a moiety as defined above that does not have any optional substituents.

A saturated, partially unsaturated or unsaturated three- to eight-membered carbocyclic ring is for example a four- to seven-membered, alternatively a five- or six-membered, saturated or unsaturated carbocyclic ring. Examples of saturated or unsaturated three- to eight-membered carbocyclic rings include phenyl, cyclopropyl, cyclobutyl, cyclopentyl, cyclohexyl, and cycloheptyl.

A saturated or unsaturated carbocyclic and heterocyclic group may condense with another saturated or heterocyclic group to form a bicyclic group, for example a saturated or unsaturated nine- to twelve-membered bicyclic carbocyclic or heterocyclic group. Bicyclic groups include naphthyl, quinolyl, 1,2,3,4-tetrahydroquinolyl, 1,4-benzoxanyl, indanyl, indolyl, and 1,2,3,4-tetrahydronaphthyl.

When a carbocyclic or heterocyclic group is substituted by two C₁-C₆alkyl groups, the two alkyl groups may combine together to form an alkylene chain, for example a C₁-C₃alkylene chain. Carbocyclic or heterocyclic groups having this crosslinked structure include bicyclo[2.2.2]octanyl and norbornanyl.

The terms “kinase inhibitor” and “inhibitor of kinase activity”, and the like, are used to identify a compound which is capable of interacting with a kinase and inhibiting its enzymatic activity.

The term “inhibiting kinase enzymatic activity” and the like is used to mean reducing the ability of a kinase to transfer a phosphate group from a donor molecule, such as adenosine tri-phosphate (ATP), to a specific target molecule (substrate). For example, the inhibition of kinase activity may be at least about 10%. In some embodiments of the invention, such reduction of kinase activity is at least about 25%, alternatively at least about 50%, alternatively at least about 75%, and alternatively at least about 90%. In other embodiments, kinase activity is reduced by at least 95% and alternatively by at least 99%. The IC₅₀ value is the concentration of kinase inhibitor which reduces the activity of a kinase to 50% of the uninhibited enzyme.

The terms “inhibitor of VEGF receptor signaling” is used to identify a compound having a structure as defined herein, which is capable of interacting with a VEGF receptor and inhibiting the activity of the VEGF receptor. In some embodiments, such reduction of activity is at least about 50%, alternatively at least about 75%, and alternatively at least about 90%. In some embodiments, activity is reduced by at least 95% and alternatively by at least 99%.

The term “inhibiting effective amount” is meant to denote a dosage sufficient to cause inhibition of kinase activity. The amount of a compound of the invention which constitutes an “inhibiting effective amount” will vary depending on the compound, the kinase, and the like. The inhibiting effective amount can be determined routinely by one of ordinary skill in the art. The kinase may be in a cell, which in turn may be in a multicellular organism. The multicellular organism may be, for example, a plant, a fungus or an animal, for example a mammal and for example a human. The fungus may be infecting a plant or a mammal, for example a human, and could therefore be located in and/or on the plant or mammal.

In an exemplary embodiment, such inhibition is specific, i.e., the kinase inhibitor reduces the ability of a kinase to transfer a phosphate group from a donor molecule, such as ATP, to a specific target molecule (substrate) at a concentration that is lower than the concentration of the inhibitor that is required to produce another, unrelated biological effect. For example, the concentration of the inhibitor required for kinase inhibitory activity is at least 2-fold lower, alternatively at least 5-fold lower, alternatively at least 10-fold lower, and alternatively at least 20-fold lower than the concentration required to produce an unrelated biological effect.

Thus, the invention provides a method for inhibiting kinase enzymatic activity, comprising contacting the kinase with an inhibiting effective amount of a compound or composition according to the invention. In some embodiments, the kinase is in an organism. Thus, the invention provides a method for inhibiting kinase enzymatic activity in an organism, comprising administering to the organism an inhibiting effective amount of a compound or composition according to the invention. In some embodiments, the organism is a mammal, for example a domesticated mammal. In some embodiments, the organism is a human.

The term “therapeutically effective amount” as employed herein is an amount of a compound of the invention, that when administered to a patient, elicits the desired therapeutic effect. The therapeutic effect is dependent upon the disease being treated and the results desired. As such, the therapeutic effect can be treatment of a disease-state. Further, the therapeutic effect can be inhibition of kinase activity. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art.

In some embodiments, the therapeutic effect is inhibition of angiogenesis. The phrase “inhibition of angiogenesis” is used to denote an ability of a compound according to the present invention to retard the growth of blood vessels, such as blood vessels contacted with the inhibitor as compared to blood vessels not contacted. In some embodiments, angiogenesis is tumor angiogenesis. The phrase “tumor angiogenesis” is intended to mean the proliferation of blood vessels that penetrate into or otherwise contact a cancerous growth, such as a tumor. In some embodiments, angiogenesis is abnormal blood vessel formation in the eye.

In an exemplary embodiment, angiogenesis is retarded by at least 25% as compared to angiogenesis of non-contacted blood vessels, alternatively at least 50%, alternatively at least 75%, alternatively at least 90%, alternatively at least 95%, and alternatively, at least 99%. Alternatively, angiogenesis is inhibited by 100% (i.e., the blood vessels do not increase in size or number). In some embodiments, the phrase “inhibition of angiogenesis” includes regression in the number or size of blood vessels, as compared to non-contacted blood vessels. Thus, a compound according to the invention that inhibits angiogenesis may induce blood vessel growth retardation, blood vessel growth arrest, or induce regression of blood vessel growth.

Thus, the invention provides a method for inhibiting angiogenesis in an animal, comprising administering to an animal in need of such treatment a therapeutically effective amount of a compound or composition of the invention. In some embodiments, the animal is a mammal, for example a domesticated mammal. In some embodiments, the animal is a human.

In some embodiments, the therapeutic effect is treatment of an ophthalmic disease, disorder or condition. The phrase “treatment of an ophthalmic disease, disorder or condition” is intended to mean the ability of a compound according to the present invention to treat (a) a disease disorder or condition caused by choroidal angiogenesis, including, without limitation, age-related macular degeneration, or (b) diabetic retinopathy or retinal edema. In some embodiments the phrase “treatment of an ophthalmic disease, disorder or condition” is intended to mean the ability of a compound according to the present invention to treat an exudative and/or inflammatory ophthalmic disease, disorder or condition, a disorder related to impaired retinal vessel permeability and/or integrity, a disorder related to retinal microvessel rupture leading to focal hemorrhage, a disease of the back of the eye, a retinal disease, or a disease of the front of the eye, or other ophthalmic disease, disorder or condition.

In some embodiments, the ophthalmic disease, disorder or condition includes but is not limited to Age Related Macular Degeneration (ARMD), exudative macular degeneration (also known as “wet” or neovascular age-related macular degeneration (wet-AMD), macular oedema, aged disciform macular degeneration, cystoid macular oedema, palpebral oedema, retinal oedema, diabetic retinopathy, Acute Macular Neuroretinopathy, Central Serous Chorioretinopathy, chorioretinopathy, Choroidal Neovascularization, neovascular maculopathy, neovascular glaucoma, obstructive arterial and venous retinopathies (e.g. Retinal Venous Occlusion or Retinal Arterial Occlusion), Central Retinal Vein Occlusion, Disseminated Intravascular Coagulopathy, Branch Retinal Vein Occlusion, Hypertensive Fundus Changes, Ocular Ischemic Syndrome, Retinal Arterial Microaneurysms, Coat's Disease, Parafoveal Telangiectasis, Hemi-Retinal Vein Occlusion, Papillophlebitis, Central Retinal Artery Occlusion, Branch Retinal Artery Occlusion, Carotid Artery Disease(CAD), Frosted Branch Angitis, Sickle Cell Retinopathy and other Hemoglobinopathies, Angioid Streaks, macular oedema occurring as a result of aetiologies such as disease (e.g. Diabetic Macular Oedema), eye injury or eye surgery, retinal ischemia or degeneration produced for example by injury, trauma or tumours, uveitis, iritis, retinal vasculitis, endophthalmitis, panophthalmitis, metastatic ophthalmia, choroiditis, retinal pigment epithelitis, conjunctivitis, cyclitis, scleritis, episcleritis, optic neuritis, retrobulbar optic neuritis, keratitis, blepharitis, exudative retinal detachment, corneal ulcer, conjunctival ulcer, chronic nummular keratitis, Thygeson keratitis, progressive Mooren's ulcer, an ocular inflammatory disease caused by bacterial or viral infection or by an ophthalmic operation, an ocular inflammatory disease caused by a physical injury to the eye, and a symptom caused by an ocular inflammatory disease including itching, flare, oedema and ulcer, erythema, erythema exsudativum multiforme, erythema nodosum, erythema annulare, scleroedema, dermatitis, angioneurotic oedema, laryngeal oedema, glottic oedema, subglottic laryngitis, bronchitis, rhinitis, pharyngitis, sinusitis, laryngitis or otitis media.

In some embodiments, the ophthalmic disease, disorder or condition is (a) a disease disorder or condition caused by choroidal angiogenesis, including, without limitation, age-related macular degeneration, or (b) diabetic retinopathy or retinal edema.

In some embodiments, the ophthalmic disease, disorder or condition includes but is not limited to age-related macular degeneration, diabetic retinopathy, retinal edema, retinal vein occlusion, neovascular glaucoma, retinopathy of prematurity, pigmentary retinal degeneration, uveitis, corneal neovascularization or proliferative vitreoretinopathy.

In some embodiments, the ophthalmic disease, disorder or condition is age-related macular degeneration, diabetic retinopathy or retinal edema.

Thus, the invention provides a method for treating an ophthalmic disease, disorder or condition in an animal, comprising administering to an animal in need of such

In some embodiments, the therapeutic effect is inhibition of retinal neovascularization. The phrase “inhibition of retinal neovascularization” is intended to mean the ability of a compound according to the present invention to retard the growth of blood vessels in the eye, for example new blood vessels originating from retinal veins, for example, to retard the growth of new blood vessels originating from retinal veins and extending along the inner (vitreal) surface of the retina.

In an exemplary embodiment, retinal neovascularization is retarded by at least 25% as compared to retinal neovascularization of non-contacted blood vessels, alternatively at least 50%, alternatively at least 75%, alternatively at least 90%, alternatively at least 95%, and alternatively, at least 99%. Alternatively, retinal neovascularization is inhibited by 100% (i.e., the blood vessels do not increase in size or number). In some embodiments, the phrase “inhibition of retinal neovascularization” includes regression in the number or size of blood vessels, as compared to non-contacted blood vessels. Thus, a compound according to the invention that inhibits retinal neovascularization may induce blood vessel growth retardation, blood vessel growth arrest, or induce regression of blood vessel growth.

Thus, the invention provides a method for inhibiting retinal neovascularization in an animal, comprising administering to an animal in need of such treatment a therapeutically effective amount of a compound or composition of the invention. In some embodiments, the animal is a mammal, for example a domesticated mammal. In some embodiments, the animal is a human.

In some embodiments, the therapeutic effect is inhibition of cell proliferation. The phrase “inhibition of cell proliferation” is used to denote an ability of a compound according to the present invention to retard the growth of cells contacted with the inhibitor as compared to cells not contacted. An assessment of cell proliferation can be made by counting contacted and non-contacted cells using a Coulter Cell Counter (Coulter, Miami, Fla.) or a hemacytometer. Where the cells are in a solid growth (e.g., a solid tumor or organ), such an assessment of cell

In an exemplary embodiment, growth of cells contacted with the inhibitor is retarded by at least 25% as compared to growth of non-contacted cells, alternatively at least 50%, alternatively at least 75%, alternatively at least 90%, alternatively at least 95%, and alternatively, at least 99%. Alternatively, cell proliferation is inhibited by 100% (i.e., the contacted cells do not increase in number). In some embodiments, the phrase “inhibition of cell proliferation” includes a reduction in the number or size of contacted cells, as compared to non-contacted cells. Thus, a compound according to the invention that inhibits cell proliferation in a contacted cell may induce the contacted cell to undergo growth retardation, to undergo growth arrest, to undergo programmed cell death (i.e., to apoptose), or to undergo necrotic cell death.

In some embodiments, the contacted cell is a neoplastic cell. The term “neoplastic cell” is used to denote a cell that shows aberrant cell growth. In some embodiments, the aberrant cell growth of a neoplastic cell is increased cell growth. A neoplastic cell may be a hyperplastic cell, a cell that shows a lack of contact inhibition of growth in vitro, a benign tumor cell that is incapable of metastasis in vivo, or a cancer cell that is capable of metastasis in vivo and that may recur after attempted removal. The term “tumorigenesis” is used to denote the induction of cell proliferation that leads to the development of a neoplastic growth.

In some embodiments, the contacted cell is in an animal. Thus, the invention provides a method for treating a cell proliferative disease or condition in an animal, comprising administering to an animal in need of such treatment a therapeutically effective amount of a compound or composition of the invention. In some embodiments, the animal is a mammal, for example a domesticated mammal. In some embodiments, the animal is a human.

The term “cell proliferative disease or condition” is meant to refer to any condition characterized by aberrant cell growth, such as abnormally increased cellular proliferation. Examples of such cell proliferative diseases or conditions amenable to inhibition and treatment include, but are not limited to, cancer. Examples of particular types of cancer include, but are not limited to, breast cancer, lung cancer, colon cancer, rectal cancer, bladder cancer, prostate cancer, leukemia and renal cancer. In some embodiments, the invention provides a method for inhibiting neoplastic cell proliferation in an animal comprising

The term “patient” as employed herein for the purposes of the present invention includes humans and other animals, for example mammals, and other organisms. Thus the compounds, compositions and methods of the present invention are applicable to both human therapy and veterinary applications. In some embodiments the patient is a mammal, for example a human.

The terms “treating”, “treatment”, or the like, as used herein cover the treatment of a disease-state in an organism, and includes at least one of: (i) preventing the disease-state from occurring, in particular, when such animal is predisposed to the disease-state but has not yet been diagnosed as having it; (ii) inhibiting the disease-state, i.e., partially or completely arresting its development; (iii) relieving the disease-state, i.e., causing regression of symptoms of the disease-state, or ameliorating a symptom of the disease; and (iv) reversal or regression of the disease-state, such as eliminating or curing of the disease. In some embodiments of the present invention the organism is an animal, for example a mammal, for example a primate, for example a human. As is known in the art, adjustments for systemic versus localized delivery, age, body weight, general health, sex, diet, time of administration, drug interaction, the severity of the condition, etc., may be necessary, and will be ascertainable with routine experimentation by one of ordinary skill in the art. In some embodiments, the terms “treating”, “treatment”, or the like, as used herein cover the treatment of a disease-state in an organism and includes at least one of (ii), (iii) and (iv) above.

Administration for non-ophthalmic diseases, disorders or conditions may be by any route, including, without limitation, parenteral, oral, sublingual, transdermal, topical, intranasal, intratracheal, or intrarectal. In some embodiments, compounds of the invention are administered intravenously in a hospital setting. In some embodiments, administration may be by the oral route.

Examples of routes of administration for ophthalmic diseases, disorders and conditions include but are not limited to, systemic, periocular, retrobulbar, intracanalicular, intravitral injection, topical (for example, eye drops), subconjunctival injection, subtenon, transcleral, intracameral, subretinal, electroporation, and sustained-release implant. Other routes of administration, other injection sites or other forms of administration for ophthalmic situations will be known or contemplated by one skilled in the art and are intended to be within the scope of the present invention.

In some embodiments of the present invention, routes of administration for ophthalmic diseases, disorders and conditions include topical, subconjunctival injection, intravitreal injection, or other ocular routes, systemically, or other methods known to one skilled in the art to a patient following ocular surgery.

In some other embodiments of the present invention, routes of administration for ophthalmic diseases, disorders and conditions include topical, intravitreal, transcleral, periocular, conjunctival, subtenon, intracameral, subretinal, subconjunctival, retrobulbar, or intracanalicular.

In some embodiments of the present invention, routes of administration for ophthalmic diseases, disorders and conditions include topical administration (for example, eye drops), systemic administration (for example, oral or intravenous), subconjunctival injection, periocular injection, intravitreal injection, and surgical implant for local delivery.

In some embodiments of the present invention, routes of administration for ophthalmic diseases, disorders and conditions include intravitreal injection, periocular injection, and sustained-release implant for local delivery.

In some embodiments of the present invention, an intraocular injection may be into the vitreous (intravitreal), under the conjunctiva (subconjunctival), behind the eye (retrobulbar), into the sclera, under the Capsule of Tenon (sub-Tenon), or may be in a depot form.

In some embodiments of the present invention, administration is local, including without limitation, topical, intravitreal, periorbital, intraocular, and other local administration to the eye, the ocular and/or periocular tissues and spaces, including without limitation, via a delivery device.

The compounds of the present invention form salts which are also within the scope of this invention.

The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., non-toxic (exhibiting minimal or no undesired toxicological effects), physiologically acceptable) salts are preferred, although other salts are also useful, e.g., in isolation or purification steps which may be employed during preparation. Salts of the compounds of the invention may be formed, for example, by reacting a compound of the present invention with an amount of acid or base, such as an equivalent amount, in a medium such as one in which the salts precipitates or in an aqueous medium followed by lyophilization.

The compounds of the present invention which contain a basic moiety, such as but not limited to an amine or a pyridine or imidazole ring, may form salts with a variety of organic and inorganic acids. Examples of acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfanotes (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of the present invention which contain an acidic moiety, such as but not limited to a carboxylic acid, may form salts with a variety of organic and inorganic bases. Examples of basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glycamides, t-butyl amines, and salts with amino acids such as arginine, lysine and the like. Basic nitrogen-containing groups may be quaternized with agents such as lower alkyl halides (e.g. methyl, ethyl, propyl and butyl chlorides, bromides and iodides), dialkyl sulfates (e.g. dimethyl, diethyl, dibuty and diamyl sulfates), long chain halides (e.g. decyl, lauryl, myristyl and stearyl chlorides, bromides and iodides), aralkyl halides (e.g. benzyl and phenethyl bromides), and others.

As used herein, the term “pharmaceutically acceptable salts” is intended to mean salts that retain the desired biological activity of the above-identified compounds and exhibit minimal or no undesired toxicological effects. Examples of such salts include, but are not limited to, salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, palmoic acid, alginic acid, polyglutamic acid, naphthalenesulfonic acid, naphthalenedisulfonic acid, methanesulfonic acid, p-toluenesulfonic acid and polygalacturonic acid. Other salts include pharmaceutically acceptable quaternary salts known by those skilled in the art, which specifically include the quaternary ammonium salt of the formula—NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

Another aspect of the invention provides compositions comprising a compound according to the present invention. For example, in some embodiments of the invention, a composition comprises a compound, or an N-oxide, hydrate, solvate, pharmaceutically acceptable salt, complex or prodrug of a compound according to the present invention present in at least about 30% enantiomeric or diastereomeric excess. In some embodiments of the invention, the compound, N-oxide, hydrate, solvate, pharmaceutically acceptable salt, complex or prodrug is present in at least about 50%, at least about 80%, or even at least about 90% enantiomeric or diastereomeric excess. In some embodiments of the invention, the compound, N-oxide, hydrate, solvate, pharmaceutically acceptable salt, complex or prodrug is present in at least about 95%, alternatively at least about 98% and alternatively at least about 99% enantiomeric or diastereomeric excess. In other embodiments of the invention, a compound, N-oxide, hydrate, solvate, pharmaceutically acceptable salt, complex or prodrug is present as a substantially racemic mixture.

Some compounds of the invention may have chiral centers and/or geometric isomeric centers (E- and Z-isomers), and it is to be understood that the invention encompasses all such optical, enantiomeric, diastereoisomeric and geometric isomers. The invention also comprises all tautomeric forms of the compounds disclosed herein. Where compounds of the invention include chiral centers, the invention encompasses the enantiomerically and/or diasteromerically pure isomers of such compounds, the enantiomerically and/or diastereomerically enriched mixtures of such compounds, and the racemic and scalemic mixtures of such compounds. For example, a composition may include a mixture of enantiomers or diastereomers of a compound of Formula (I) in at least about 30% diastereomeric or enantiomeric excess. In some embodiments of the invention, the compound is present in at least about 50% enantiomeric or diastereomeric excess, in at least about 80% enantiomeric or diastereomeric excess, or even in at least about 90% enantiomeric or diastereomeric excess. In some embodiments of the invention, the compound is present in at least about 95%, alternatively in at least about 98% enantiomeric or diastereomeric excess, and alternatively in at least about 99% enantiomeric or diastereomeric excess.

The chiral centers of the present invention may have the S or R configuration. The racemic forms can be resolved by physical methods, such as, for example, fractional crystallization, separation or crystallization of diastereomeric derivates or separation by chiral column chromatography. The individual optical isomers can be obtained either starting from chiral precursors/intermediates or from the racemates by any suitable method, including without limitation, conventional methods, such as, for example, salt formation with an optically active acid followed by crystallization.

The present invention also includes prodrugs of compounds of the invention. The term “prodrug” is intended to represent a compound covalently bonded to a carrier, which prodrug is capable of releasing the active ingredient when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of the invention include compounds wherein a hydroxy, an amino, a carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of the present invention), amides (e.g., trifluoroacetylamino, acetylamino, and the like), and the like.

The compounds of the invention may be administered, for example, as is or as a prodrug, for example in the form of an in vivo hydrolyzable ester or in vivo hydrolyzable amide. An in vivo hydrolyzable ester of a compound of the invention containing a carboxy or hydroxy group is, for example, a pharmaceutically acceptable ester which is hydrolyzed in the human or animal body to produce the parent acid or alcohol. Suitable pharmaceutically acceptable esters for carboxy include C₁-C₆alkoxymethyl esters (e.g., methoxymethyl), C₁-C₆alkanoyloxymethyl esters (e.g., for example pivaloyloxymethyl), phthalidyl esters, C₃-C₈cycloalkoxycarbonyloxy-C₁-C₆alkyl esters (e.g., 1-cyclohexylcarbonyloxyethyl); 1,3-dioxolen-2-onylmethyl esters (e.g., 5-methyl-1,3-dioxolen-2-onylmethyl; and C₁-C₆alkoxycarbonyloxyethyl esters (e.g., 1-methoxycarbonyloxyethyl) and may be formed at any appropriate carboxy group in the compounds of this invention.

An in vivo hydrolyzable ester of a compound of the invention containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolyzable ester forming groups for hydroxy include alkanoyl, benzoyl, phenylacetyl and substituted benzoyl and phenylacetyl, alkoxycarbonyl (to give alkyl carbonate esters), dialkylcarbamoyl and N—(N,N-dialkylamino ethyl)-N-alkylcarbamoyl (to give carbamates), N,N-dialkylaminoacetyl and carboxyacetyl. Examples of substituents on benzoyl include morpholino and piperazino linked from a ring nitrogen atom via a methylene group to the 3- or 4-position of the benzoyl ring. A suitable value for an in vivo hydrolyzable amide of a compound of the invention containing a carboxy group is, for example, a N—C₁-C₆alkyl or N,N-di-C₁-C₆alkyl amide such as N-methyl, N-ethyl, N-propyl, N,N-dimethyl, N-ethyl-N-methyl or N,N-diethyl amide.

Upon administration to a subject, the prodrug undergoes chemical conversion by metabolic or chemical processes to yield a compound of the present invention.

The present invention is also directed to solvates and hydrates of the compounds of the present invention. The term “solvate” refers to a molecular complex of a compound with one or more solvent molecules in a stoichiometric or non-stoichiometric amount. A molecular complex of a compound or moiety of a compound and a solvent can be stabilized by non-covalent intra-molecular forces such as, for example, electrostatic forces, van der Waals forces, or hydrogen bonds. Those skilled in the art of organic chemistry will appreciate that many organic compounds can form such complexes with solvents in which they are obtained, prepared or synthesized, or from which they are precipitated or crystallized. The term “hydrate” refers to a complex in which the one or more solvent molecules are water and includes monohydrates, hemi-hydrates, dihydrates, hexahydrates, and the like. The meaning of the words “solvate” and “hydrate” are well known to those skilled in the art. Techniques for the preparation of solvates are well established in the art (see, for example, Brittain, Polymorphism in Pharmaceutical solids. Marcel Dekker, New York, 1999; Hilfiker, Polymorphism in the Pharmaceutical Industry, Wiley, Weinheim, Germany, 2006).

In some embodiments of this aspect, the solvent is an inorganic solvent (for example, water). In some embodiments of this aspect, the solvent is an organic solvent (such as, but not limited to, alcohols, such as, without limitation, methanol, ethanol, isopropanol, and the like, acetic acid, ketones, esters, and the like). In certain embodiments, the solvent is one commonly used in the pharmaceutical art, is known to be innocuous to a recipient to which such solvate is administered (for example, water, ethanol, and the like) and in preferred embodiments, does not interfere with the biological activity of the solute.

Throughout the specification, embodiments of one or more chemical substituents are identified. Also encompassed are combinations of various embodiments. For example, the invention describes some embodiments of D in the compounds and describes some embodiments of group G. Thus, as an example, also contemplated as within the scope of the invention are compounds in which examples of D are as described and in which examples of group G are as described.

### Compounds

According to one aspect, the invention is directed to compounds having the Formula (I):

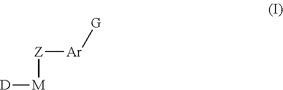

including N-oxides, hydrates, solvates, tautomers, pharmaceutically acceptable salts, prodrugs and complexes thereof, and racemic and scalemic mixtures, diastereomers and enantiomers thereof, wherein,
D is selected from the group consisting of an aromatic, heteroaromatic, cycloalkyl or heterocyclic ring system, C₁-C₆alkyl-heterocyclyl-C(O)—, C₁-C₆alkyl-heterocyclyl-C₁-C₆alkyl-N(R⁶)—C(O)—, (R⁶)(R⁶)N—C(O)—O-heterocyclyl-C(O)—, heterocyclyl-C(O)—, PivO-heterocyclyl-C(O)—, C₁-C₆alkyl-O—C(O)-heterocyclyl-C(O)—, C₁-C₆alkyl-C(O)—N(R⁶)-heterocyclyl-C(O)—, (C₁-C₆alkyl)(Box)N-heterocyclyl-C(O)—, HO-heterocyclyl-C(O)—, HO—C(O)-heterocyclyl-C(O)—, C₁-C₆alkyl-C(O)—O-heterocyclyl-C(O)—, (R⁶)(R⁶)N—C₁-C₆alky-N(R⁶)—C(O)-heterocyclyl-C(O)—, C₁-C₆alkyl-heterocyclyl-C(O)-heterocyclyl-C(O)— and (R⁶)(R⁶)N-heterocyclyl-C(O)—, wherein each of the aromatic, heteroaromatic, cycloalkyl and heterocyclic groups is optionally substituted with 1 or more independently selected R³⁸;
M is an optionally substituted fused heterocyclic moiety;
Z is selected from the group consisting of —O—, —S(O)₀₋₂— and —NR⁵—, wherein R⁵ is selected from the group consisting of H, optionally substituted C₁-C₅alkyl, an optionally substituted (C₁-C₅)acyl and C₁-C₆ alkyl-O—C(O), wherein C₁-C₆ alkyl is optionally substituted;
Ar is a group of the formulas C-1 or C-2,

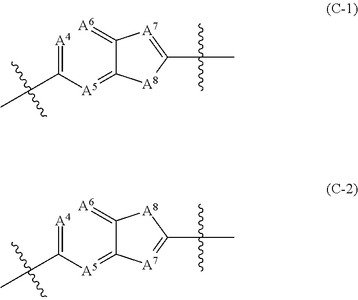

wherein A⁴, A⁵, A⁶ and A⁷ are independently selected from the group consisted of —CH— or N, and A⁸ is O, S(O)₀₋₂, CH₂, NH, NC₁₋₄-alkyl or NC₁₋₄-cycloalkyl;
G is a group B-L, wherein
   B is selected from the group consisting of O, S(O)₀₋₂, CH₂, NH, NC₁₋₄-alkyl or NC₁₋₄-cycloalkyl;
   L is independently selected from the group consisted of cycloalkyl, heterocyclyl, aryl or heteroaryl wherein the cycloalkyl, heterocyclyl, aryl or heteroaryl can be optionally substituted by 1-3 R²⁰;
wherein
R³⁸ is selected from the group consisting of C₂-C₆alkynyl-heterocyclyl, H(O)C— and C₁-C₆alkyl-C(O)—O—C₁-C₆alkyl-C(O)—, R³⁷O—C₁-C₆alkyl-C(O)-heterocyclyl-C₁-C₆alkyl-, R³⁷O—(CH₂)₁₋₆—N(A)-(CH₂)₁₋₄—, C₁-C₆alkyl-S(O)₂—(CH₂)₂—N(A)-CH₂—, R³⁷O—(CH₂)ⱼ—[(CH₂)ᵢO]ₓ—(CH₂)ᵢ₁—N(A)-(CH₂)ⱼ₁—, R³⁷O—C(O)—C₀-C₆alkyl-heterocyclyl-CH₂—, R³⁷O—(CH₂)ⱼ—[(CH₂)ᵢO]ₓ—(CH₂)ᵢ₁—N(R³⁹)—C(O)—, R³⁷—O—C(O)—C₁-C₆alkyl-heterocyclyl-C(O)—, HOOC—C₁-C₆alkyl-N(A)-CH₂—, (HOOC)(NR⁹R¹⁰)—C₁-C₆alkyl-N(A)-CH₂—, R³⁷O—C(O)—C₁-C₆alkyl-C(O)—, (R⁹)(R¹⁰)N—C₁-C₆alkyl-C(O)-heterocyclyl-CH₂—, cycloalkyl-N(R³⁹)—C(O)—O—C₁-C₆alkyl-, R³⁷—O—C₁-C₆alkyl-O—C₁-C₆alkyl-C(O)—, (R⁹)(R¹⁰)N—C(O)—C₁-C₆alkyl-heterocyclyl-CH₂—, (R⁹)(R¹⁰)N—C₁-C₆alkyl-C(O)—O—C₁-C₆alkyl-heterocyclyl-CH₂—, NC—C₁-C₆alkyl-heterocyclyl-CH₂—, F₃C—C₁-C₆alkyl-heterocyclyl-CH₂—, C₁-C₆alkyl-C(O)—O—C₁-C₆alkyl-C(O)-(5 to 10-membered heterocyclyl)-C₁-C₆alkyl-, (optionally substituted 8- to 10-membered fused heterocyclyl)-C₁-C₆alkyl-, F-heterocyclyl-C₁-C₆alkyl-, heteroaryl-C₁-C₆alkyl-heterocyclyl-C₁-C₆alkyl-, R³⁷—C₁-C₆alkyl-O—C₁-C₆alkyl-heterocyclyl-C₁-C₆alkyl, R³⁷O—C(O)—C₁-C₆alkyl-O-heterocyclyl-C₁-C₆alkyl-, R³⁷O—C(O)—C₁-C₆alkyl-heterocyclyl-C₁-C₆alkyl-, heterocyclyl-C₁-C₆alkyl-O-aryl-N(R⁶)—C₁-C₆alkyl-, (heteroaryl substituted with one or more C₁-C₆alkyl)-N(R⁶)—C₁-C₆alkyl-, (C₁-C₆alkyl)₂N—C₁-C₆alkyl-aryl-N(R⁶)—C₁-C₆alkyl-, (C₁-C₆alkyl)₂N—C₁-C₆alkyl-C(O)-aryl-N(R⁶)—C₁-C₆alkyl-, heterocyclyl-C₁-C₆alkyl-O-aryl-N(R⁶)—C₁-C₆alkyl-, (R⁶)₂N-heterocyclyl-C₁-C₆alkyl-, C₁-C₆alkyl-C(O)—N(R⁶)-heterocyclyl-C₁-C₆alkyl-, C₁-C₆alkylC(O)—O—C₁-C₆alkyl-C(O)—N(R⁶)-heterocyclyl-C₁-C₆alkyl-, R³⁷O—C₁-C₆alkyl-C(O)—N(R⁶)-heterocyclyl-C₁-C₆alkyl-, heteroaryl-C₁-C₆alkyl-C(O)—N(R⁶)-heterocyclyl-C₁-C₆alkyl-, C₁-C₆alkyl-S(O)₂—N(R⁶)-heterocyclyl-C₁-C₆alkyl-, C₁-C₆alkyl-O—C(O)—N(R⁶)-heterocyclyl-C₁-C₆alkyl-, C₁-C₆alkyl-N(R⁶)—C(O)—N(R⁶)-heterocyclyl-C₁-C₆alkyl-, C₁-C₆alkyl-heterocyclyl-C(O)—N(R⁶)-heterocyclyl-C₁-C₆alkyl-, R³⁷O—C₁-C₆alkyl-N(R⁶)—C(O)—N(R⁶)-heterocyclyl-C₁-C₆alkyl-, (heterocyclyl optionally substituted with one or more C₁-C₆alkyl)-C₁-C₆alkyl-, (C₁-C₆alkyl)₂N—C₁-C₆alkyl-, C₁-C₆alkyl-heterocyclyl-C(O)—C₁-C₆alkyl-, heterocyclyl-C(O)—C₁-C₆alkyl-, C₁-C₆alkyl-O—C(O)—C₁-C₆alkyl-, C₁-C₆alkyl-O—C(O)—C₁-C₆alkyl-heteroaryl-N(R⁶)—C(O)—C₁-C₆alkyl-, (C₁-C₆alkyl)₂N-heterocyclyl-C(O)—C₁-C₆alkyl-, heteroaryl-C₁-C₆alkyl-N(R⁶)—C(O)—C₁-C₆alkyl-, (Boc)(H)N-heterocyclyl-C(O)—C₁-C₆alkyl-, C₁-C₆alkyl-O—C(O)-heterocyclyl-C(O)—C₁-C₆alkyl-, Boc-heterocyclyl-C(O)—C₁-C₆alkyl-, Ac—O—C₁-C₆alkyl-C(O)-heterocyclyl-C(O)—C₁-C₆alkyl-, R³⁷O—C₁-C₆alkyl-C(O)-heterocyclyl-C(O)—C₁-C₆alkyl-, (Boc)(H)N—C₁-C₆alkyl-C(O)-heterocyclyl-C(O)—C₁-C₆alkyl-, NH₂—C₁-C₆alkyl-C(O)-heterocyclyl-C(O)—C₁-C₆alkyl-, (C₁-C₆alkyl)(H)N—C(O)-heterocyclyl-C(O)—C₁-C₆alkyl-, NH₂-heterocyclyl-C(O)—C₁-C₆alkyl-, R³⁷O—C₁-C₆alkyl-O—C₁-C₆alkyl-heterocyclyl-C(O)—, C₁-C₆alkyl-O—C(O)—N(R⁶)-heterocyclyl-C(O)—, (R⁶)(R⁶)N-heterocyclyl-C(O)—, (R⁶)(R⁶)N-heterocyclyl-C₁-C₆alkyl-, heterocyclyl-O—C₁-C₆alkyl-, C₁-C₆alkyl-N(R⁶)—C(O)—N(R⁶)-heterocyclyl-C(O)—, (R⁶)(R⁶)N—C(O)-heterocyclyl-O—C₁-C₆alkyl-, C₂-C₆alkenyl-C(O)—N(R⁶)-heterocyclyl-C₁-C₆alkyl-, R³⁷O—C₁-C₆alkyl-C(O)-heterocyclyl-O—C₁-C₆alkyl-, R^{37a}—C₁-C₆alkyl-N(R⁶)-heterocylcyl-C₁-C₆alkyl-, R³⁷O—(CH₂)ⱼ-[(CH₂)ᵢO]ₓ—C₁-C₆alkyl-N(R⁶)-heterocyclyl-C₁-C₆alkyl-, halogen-C₁-C₆alkyl-heterocyclyl-C₁-C₆alkyl-, halogen-C₁-C₆alkyl-N(R⁶)-heterocyclyl-C₁-C₆alkyl-, R³⁷O—C(O)—C₁-C₆alkyl-N(R⁶)-heterocyclyl-C₁-C₆alkyl-, R³⁷—O—C(O)—C₁-C₆alkyl-N(R⁶)—C(O)—N(R⁶)-heterocyclyl-C₁-C₆alkyl-, (C₁-C₆alkyl)(H)N—C(O)-heterocyclyl-N[C₁-C₆alkyl-C(O)—OH]-C₁-C₆alkyl-, C₁-C₆alkyl-O—C(O)-heterocylcyl-C₁-C₆alkyl-, HO—C(O)-heterocyclyl-C₁-C₆alkyl-, C₁-C₆alkyl-heterocyclyl-C(O)-heterocyclyl-C₁-C₆alkyl-, R³⁷O—C₁-C₆alkyl-N(R⁶)—C(O)-heterocyclyl-C₁-C₆alkyl-, (R⁶)(R⁶)N—C₁-C₆alkyl-N(R⁶)—CO)-heterocyclyl-C₁-C₆alkyl-, (C₁-C₆alkyl)(C₁-C₆alkyl)N-heterocyclyl-C₁-C₆alkyl-, R³⁷O—C₁-C₆alkyl-C(O)-[(C₁-C₆alkyl)(C₁-C₆alkyl)heterocyclyl]-C₁-C₆alkyl-, C₂-C₆alkenyl-C(O)-[(C₁-C₆alkyl)(C₁-C₆alkyl)heterocyclyl]-C₁-C₆alkyl-, R³⁷O—C₁-C₆alkyl-[(C₁-C₆alkyl)(C₁-C₆alkyl)heterocyclyl]-C₁-C₆alkyl-, C₁-C₆alkyl-O—C₁-C₆alkyl-N[C(O)—NH—C₁-C₆alkyl]-C₁-C₆alkyl-, C₁-C₆alkyl-O—C₁-C₆alkyl-N[C(O)—C₁-C₆alkyl]-C₁-C₆alkyl-, C₁-C₆alkyl-O—C₁-C₆alkyl-[C(O)—C₁-C₆alkyl-OH]-C₁-C₆alkyl-, R³⁷O—C(O)—C₁-C₆alkyl-C(O)-heterocyclyl-C₁-C₆alkyl-, R³⁷O—C(O)—C₁-C₆alkyl-heterocyclyl-C₁-C₆alkyl-, spiro-heterocyclyl-C₁-C₆alkyl-, R³⁷O—C₁-C₆alkyl-C(O)-spiro-heterocyclyl-C₁-C₆alkyl-, R³⁷O—C₁-C₆alkyl-C(O)-heterocyclyl-C₁-C₆alkyl-, C₁-C₆alkyl-heterocyclyl-C₁-C₆alkyl-, C₁-C₆alkyl-C(O)—O—C₁-C₆alkyl-C(O)-heterocyclyl-C₁-C₆alkyl-, heterocyclyl-C₁-C₆alkyl-C(O)-heterocyclyl-C₁-C₆alkyl-, (R⁶)(R⁶)N—C₁-C₆alkyl-C(O)-heterocyclyl-C₁-C₆alkyl-, heterocyclyl-C(O)-heterocyclyl-C₁-C₆alkyl-, (R⁶)(R⁶)N—C₂-C₆alkenyl-C(O)-heterocyclyl-C₁-C₆alkyl-, heterocyclyl-C₂-C₈alkenyl-C(O)-heterocyclyl-C₁-C₆alkyl-, (R⁶)(R⁶)N—C₁-C₆alkyl-N(R⁶)—C₁-C₆alkyl-C(O)-heterocyclyl-C₁-C₆alkyl-, heterocyclyl-C(O)—, (R⁶)(R⁶)N—C(O)-heterocyclyl-C₁-C₆alkyl-, R³⁷O—C(O)—C₁-C₆alkyl-N(R⁶)—C(O)-heterocyclyl-C₁-C₆alkyl-, C₂-C₆alkenyl-C(O)—O—C₁-C₆alkyl-N(R⁶)—C(O)-heterocyclyl-C₁-C₆alkyl-, (R⁶)(R⁶)N—C(O)-heterocyclyl-C(O)—, R³⁷O—C₁-C₆alkyl-N(R⁶)—C(O)-heterocyclyl-C₁-C₆alkyl-, R³⁷O—C₁-C₆alkyl-heterocyclyl-C₁-C₆alkyl-(heterocyclyl)-, R³⁷O—C(O)—C₁-C₆alkyl-heterocyclyl-C(O)—, R³⁷O—C₁-C₆alkyl-heterocyclyl-C(O)—, R³⁷O—C₁-C₆alkyl-C(O)-heterocyclyl-C(O)—, C₁-C₆alkyl-O—C(O)—N(R⁶)—C₁-C₆alkyl-C(O)—O—C₁-C₆alkyl-C(O)-heterocyclyl-C₁-C₆alkyl-, R³⁷O—(CH₂)ₙ[(CH₂)ᵢO]ₓ—C₁-C₆alkyl-N(R⁶)—C(O)-heterocyclyl-C₁-C₆alkyl-, HO-heterocyclyl-C₁-C₆alkyl-, R³⁷O-cycloalkyl-C(O)-heterocyclyl-C₁-C₆alkyl- and R³⁷O—(CH₂)ₙ[(CH₂)ᵢO]ₓ—C₁-C₆alkyl-C(O)—N(R⁶)-heterocyclyl-C₁-C₆alkyl;
A is selected from the group consisting of —C(O)—C₁-C₆alkyl-N(R³⁹)—C(O)—C₁-C₆alkyl-N(R⁹)(R¹⁰), —C(O)—N(R³⁹)—C₁-C₆alkyl, —C(═NR³⁷)—C₁-C₆alkyl, —C(O)—(CH₂)ₙ—S(O)₂—C₁-C₆alkyl, —C(O)—N(R³⁹)-cycloalkyl, —C(O)—N(R⁹)(R¹⁰), (R^{37a}O)(R^{37a}O)P(O)O—C₁-C₆alkyl-C(O)—, —C(═NR³⁷)—H and —C₁-C₆alkyl-CF₃;
each R⁶ is independently H or C₁-C₆alkyl;
R³⁷ is selected from the group consisting of H, C₁-C₆alkyl and C₃-C₁₀cycloalkyl;
R^{37a} is selected from the group consisting of H, C₁-C₆alkyl and C₃-C₁₀cycloalkyl;
j is an integer ranging from 0 to 4, alternatively 0 to 2;
i is 2 or 3;
x is an integer ranging from 0 to 6, alternatively 2 or 3;
i1 is 2 or 3;
j1 is an integer ranging from 0 to 4, alternatively 1 or 2;
n is an integer ranging from 0 to 4;
R³⁹ is selected from the group consisting of H, —OH, C₁-C₆ alkyl, C₃-C₁₀ cycloalkyl, —(CH₂)ₙ₂(C₆-C₁₀ aryl), —(CH₂)ₙ₂(C₅-C₁₀ heteroaryl), —(CH₂)ₙ₂(5-10 membered heterocyclyl), —(CH₂)ₙ₂O—(CH₂)ᵢ₂OR³⁷ and —(CH₂)ₙ₂OR³⁷, wherein the alkyl, aryl, heteroaryl and heterocyclyl moieties of the foregoing R³⁹ groups are optionally substituted;
R⁹ is selected from the group consisting of H, —OH, C₁-C₆ alkyl, C₃-C₁₀ cycloalkyl, —(CH₂)ₙ₃(C₆-C₁₀ aryl), —(CH₂)ₙ₃(C₅-C₁₀ heteroaryl), —(CH₂)ₙ₃(5-10 membered heterocyclyl), —(CH₂)ₙ₃O(CH₂)ᵢ₃OR³⁷ and —(CH₂)ₙ₃OR³⁷, wherein the alkyl, aryl, heteroaryl and heterocyclyl moieties of the foregoing R⁹ groups are optionally substituted;
R¹⁰ is selected from the group consisting of H, —OH, C₁-C₆ alkyl, C₃-C₁₀ cycloalkyl, —(CH₂)ₙ₄(C₆-C₁₀ aryl), —(CH₂)ₙ₄(C₅-C₁₀ heteroaryl), —(CH₂)ₙ₄(5-10 membered heterocyclyl), —(CH₂)ₙ₄O(CH₂)ᵢ₄OR³⁷ and —(CH₂)ₙ₄OR³⁷, wherein the alkyl, aryl, heteroaryl and heterocyclyl moieties of the foregoing R¹⁰ groups are optionally substituted;
R²⁰ is selected from the group consisting of —H, halogen, trihalomethyl, —CN, —NO₂, —NH₂, —OR³, C₃₋₆-cycloalkyl, C₁₋₆-alkoxy, C₃₋₆-cycloalkoxy, CF₃, CCl₃, —OCF₃, —NR³R⁴, —S(O)₀₋₂R³, —S(O)₂NR³R³, —C(O)OR³, —C(O)NR³R³, —N(R³)SO₂R³, —N(R³)C(O)R³, —N(R³)C(O)OR³, —C(O)R³, —C(O)SR³, C₁-C₄ alkoxy, C₁-C₄ alkylthio, —O(CH₂)ₙ₆aryl, —O(CH₂)ₙ₆heteroaryl, —(CH₂)ₙ₆(aryl), —(CH₂)ₙ₆(heteroaryl), C₁-C₆ alkyl, C₂-C₆ alkenyl, C₂-C₆ alkynyl, —CH₂(CH₂)₀₋₄-T², an optionally substituted C₁₋₄ alkylcarbonyl, C₁₋₄ alkoxy, an amino optionally substituted by C₁₋₄ alkyl optionally substituted by C₁₋₄ alkoxy, —(CH₂)ₙ₆P(═O)(C₁-C₆alkyl)₂, a saturated or unsaturated three- to seven-membered cycloalkyl or heterocyclic group, —SiMe₃ and —SbF₅;
n2 is an integer ranging from 0 to 6;
i2 is an integer ranging from 2 to 6;
n3 is an integer ranging from 0 to 6;
i3 is an integer ranging from 2 to 6;
n4 is an integer ranging from 0 to 6; and
i4 is an integer ranging from 2 to 6
each R³ is independently selected from the group consisting of —H and R⁴;
R⁴ is selected from the group consisting of a (C₁-C₆)alkyl, an aryl, a lower arylalkyl, a heterocyclyl and a lower heterocyclyl-alkyl, each of which is optionally substituted, or
R³ and R⁴, taken together with a common nitrogen to which they are attached, form an optionally substituted five- to seven-membered heterocyclyl, the optionally substituted five- to seven-membered heterocyclyl optionally containing at least one additional annular heteroatom selected from the group consisting of N, O, S and P;
T² is selected from the group consisting of —OH, —OMe, —OEt, —NH₂, —NHMe, —NMe₂, —NHEt and —NEt₂, and wherein the aryl, heteroaryl, C₁-C₆ alkyl, C₂-C₆ alkenyl, and C₂-C₆ alkynyl are optionally substituted; and
n6 is an integer ranging from 0 to 6.

In some embodiments of the first aspect, the compounds have the Formula (I), wherein D is -aryl or -heteroaryl each of which is substituted with 1 or more R³⁸.

In some embodiments of the first aspect, the compounds have the Formula (I), wherein D is selected from the group consisting of

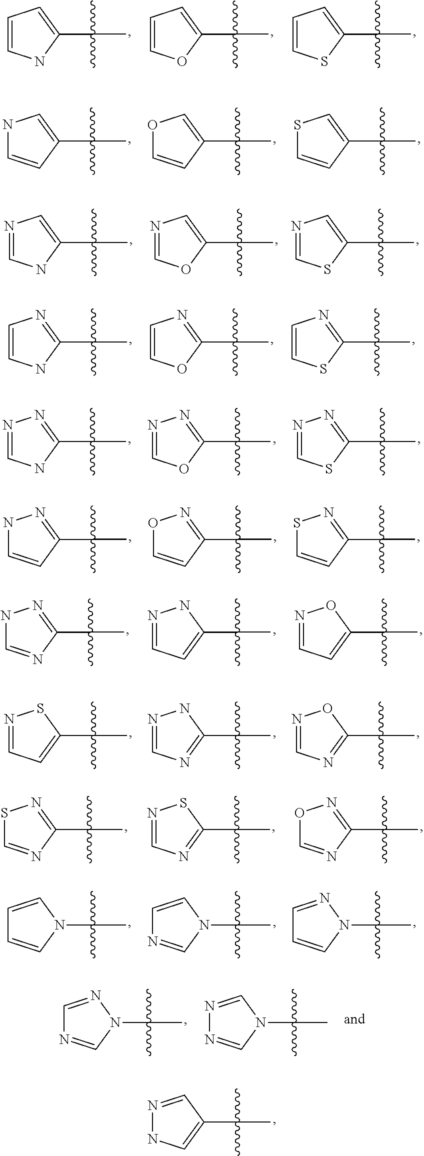

wherein the members of said group are substituted by 1 or more R³⁸.

In some embodiments of the first aspect, the compounds have the Formula (I), wherein D is selected from the group consisting of

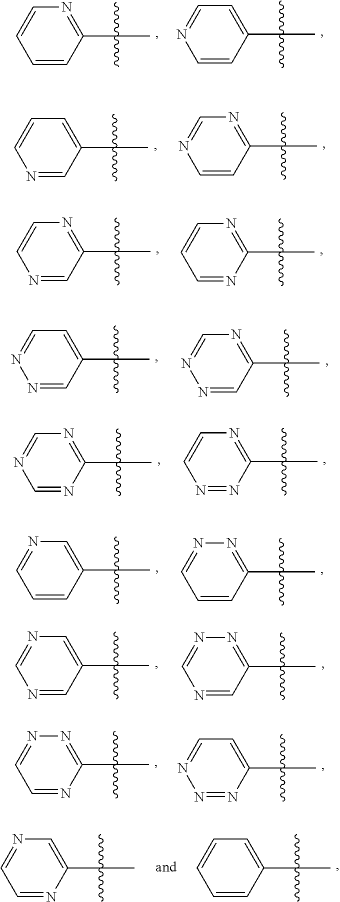

wherein the members of said group are substituted with 1 or more R³⁸.

In some embodiments of the first aspect, the compounds have the Formula (I), wherein D is selected from the group consisting of phenyl, pyridine, imidazole, pyrazole and tetrahydropyridine substituted with one R³⁸, wherein when D is imidazole said imidazole is further optionally substituted with one C₁-C₆alkyl.

In some embodiments of the first aspect, the compounds have the Formula (I), wherein D is phenyl or pyridine substituted with one R³⁸.

In some embodiments of the first aspect, the compounds have the Formula (I), wherein D is pyridine substituted with one R³⁸.

In some embodiments of the first aspect, the compounds have the Formula (I), wherein D is pyridine substituted with one R³⁸, wherein R³⁸ is selected from the group consisting of R³⁷O—(CH₂)₁₋₆—N(A)-(CH₂)₁₋₄—, R³⁷O—(CH₂)ⱼ—[(CH₂)ᵢO]ₓ—(CH₂)ᵢ₁—N(A)-(CH₂)ⱼ₁—, R³⁷O—C(O)—C₀-C₆alkyl-heterocyclyl-CH₂—, R³⁷O—(CH₂)ⱼ—[(CH₂)ᵢO]ₓ—(CH₂)ᵢ₁—N(R³⁹)—C(O)—, R³⁷—O—C(O)—C₁-C₆ alkyl-heterocyclyl-C(O)—, C₀-C₆alkyl-heterocyclyl-C₀-C₆alkyl-heterocyclyl-C(O)—, (R⁹)(R¹⁰)N—C₁-C₆ alkyl-C(O)-heterocyclyl-CH₂—, (R⁹)(R¹⁰)N—C(O)—C₁-C₆alkyl-heterocyclyl-CH₂—, (R⁹)(R¹⁰)N—C₁-C₆alkyl-C(O)—O—C₁-C₆alkyl-heterocyclyl-CH₂—, NC—C₁-C₆alkyl-heterocyclyl-CH₂—, F₃C—C₁-C₆ alkyl-heterocyclyl-CH₂— and N(R⁹)(R¹⁰)N—C₁-C₆ alkyl-C(O)—O—C₁-C₆alkyl-C(O)-heterocyclyl-CH₂—.

In some embodiments of the first aspect, the compounds have the Formula (I), wherein D is pyridine substituted with one R³⁸, wherein R³⁸ is selected from the group consisting of R³⁷O—(CH₂)₁₋₆—N(A)-(CH₂)₁₋₄—, R³⁷O—(CH₂)ⱼ—[(CH₂)ᵢO]ₓ—(CH₂)ᵢ₁—N(A)-(CH₂)ⱼ₁—, R³⁷O—C(O)—C₀-C₆alkyl-heterocyclyl-CH₂—, R³⁷O—(CH₂)ⱼ—[(CH₂)ᵢO]ₓ—(CH₂)ᵢ₁—N(R³⁹)—C(O)—, R³⁷—O—C(O)—C₁-C₆ alkyl-heterocyclyl-C(O)—, (R⁹)(R¹⁰)N—C₁-C₆ alkyl-C(O)-heterocyclyl-CH₂—, (R⁹)(R¹⁰)N—C(O)—C₁-C₆alkyl-heterocyclyl-CH₂—, (R⁹)(R¹⁰)N—C₁-C₆alkyl-C(O)—O—C₁-C₆alkyl-heterocyclyl-CH₂—, NC—C₁-C₆alkyl-heterocyclyl-CH₂—, F₃C—C₁-C₆alkyl-heterocyclyl-CH₂— and N(R⁹)(R¹⁰)N—C₁-C₆alkyl-C(O)—O—C₁-C₆alkyl-C(O)-heterocyclyl-CH₂—.

In some embodiments of the first aspect, the compounds have the Formula (I), wherein D is pyridine substituted with one R³⁸, wherein R³⁸ is R³⁷O—(CH₂)₁₋₆—N(A)-(CH₂)₁₋₄— or R³⁷O—(CH₂)ⱼ—[(CH₂)ᵢO]ₓ—(CH₂)ᵢ₁—N(A)-(CH₂)ⱼ₁—, and A is selected from the group consisting of —C(O)—C₁-C₆alkyl-N(R³⁹)—C(O)—C₁-C₆alkyl-N(R⁹)(R¹⁰), —C(O)—N(R³⁹)—C₁-C₆alkyl, —C(═NR³⁷)—C₁-C₆alkyl, —C(O)—(CH₂)ₙ—S(O)₂—C₁-C₆alkyl, —C(O)—N(R⁹)(R¹⁰) and (R³⁷O)(R^{37a}O)P(O)O—C₁-C₆alkyl-C(O)—.

In some embodiments of the first aspect, the compounds have the Formula (I), wherein D is pyridine substituted with one R³⁸, wherein R³⁸ is R³⁷O—(CH₂)₁₋₆—N(A)-(CH₂)₁₋₄—, alternatively R³⁷O—(CH₂)₂—N(A)-(CH₂)—, alternatively R³⁷O—(CH₂)₂—N(A)-(CH₂)₂—.

In some embodiments of the first aspect, the compounds have the Formula (I), wherein D is pyridine substituted with one R³⁸, wherein R³⁸ is R³⁷O—(CH₂)₁₋₆—N(A)-(CH₂)₁₋₄—, and A is selected from the group consisting of —C(O)—C₁-C₆alkyl-N(R³⁹)—C(O)—C₁-C₆alkyl-N(R⁹)(R¹⁰), —C(O)—N(R³⁹)—C₁-C₆alkyl, —C(═NR³⁷)—C₁-C₆alkyl, —C(O)—(CH₂)ₙ—S(O)₂—C₁-C₆alkyl, —C(O)—N(R⁹)(R¹⁰) and (R³⁷O)(R^{37a}O)P(O)O—C₁-C₆alkyl-C(O)—.

In some embodiments of the first aspect, the compounds have the Formula (I), wherein D is pyridine substituted with one R³⁸, wherein R³⁸ is R³⁷O—(CH₂)₂—N(A)-(CH₂)—, and A is selected from the group consisting of —C(O)—C₁-C₆alkyl-N(R³⁹)—C(O)—C₁-C₆alkyl-N(R⁹)(R¹⁰), —C(O)—N(R³⁹)—C₁-C₆alkyl, —C(═NR³⁷)—C₁-C₆alkyl, —C(O)—(CH₂)ₙ—S(O)₂—C₁-C₆alkyl, —C(O)—N(R⁹)(R¹⁰) and (R³⁷O)(R^{37a}O)P(O)O—C₁-C₆alkyl-C(O)—.

In some embodiments of the first aspect, the compounds have the Formula (I), wherein D is pyridine substituted with one R³⁸, wherein R³⁸ is R³⁷O—(CH₂)₂—N(A)-(CH₂)₂—, and A is —C(O)—N(R³⁹)—C₁-C₆alkyl.

In some embodiments of the first aspect, the compounds have the Formula (I), wherein D is pyridine substituted with one R³⁸, wherein R³⁸ is R³⁷O—(CH₂)₂—N(A)-(CH₂)—, and A is —C(O)—N(R³⁹)—C₁-C₆alkyl.

In some embodiments of the first aspect, the compounds have the Formula (I), wherein D is pyridine substituted with one R³⁸, wherein R³⁸ is R³⁷O—(CH₂)₂—N(A)-(CH₂)—, and A is —C(O)—H.

In some embodiments of the first aspect, the compounds have the Formula (I), wherein D is pyridine substituted with one R³⁸, wherein R³⁸ is R³⁷O—(CH₂)ⱼ—[(CH₂)ᵢO]ₓ—(CH₂)ᵢ₁—N(A)-(CH₂)ⱼ₁—.

In some embodiments of the first aspect, the compounds have the Formula (I), wherein D is pyridine substituted with one R³⁸, wherein R³⁸ is R³⁷O—(CH₂)ⱼ—[(CH₂)ᵢO]ₓ—(CH₂)ᵢ₁—N(A)-(CH₂)ⱼ₁—, and A is —C(O)—N(R³⁹)—C₁-C₆alkyl,

In some embodiments of the first aspect, the compounds have the Formula (I), wherein D is pyridine substituted with one R³⁸, wherein R³⁸ is R³⁷O—C(O)—C₀-C₆alkyl-heterocyclyl-CH₂—.

In some embodiments of the first aspect, the compounds have the Formula (I), wherein D is pyridine substituted with one R³⁸, wherein R³⁸ is R³⁷O—(CH₂)ⱼ—[(CH₂)ᵢO]ₓ—(CH₂)ᵢ₁—N(R³⁹)—C(O)—.

In some embodiments of the first aspect, the compounds have the Formula (I), wherein D is pyridine substituted with one R³⁸, wherein R³⁸ is R³⁷—O—C(O)—C₁-C₆alkyl-heterocyclyl-C(O)—.

In some embodiments of the first aspect, the compounds have the Formula (I), wherein D is pyridine substituted with one R³⁸, wherein R³⁸ is C₀-C₆alkyl-heterocyclyl-C₀-C₆alkyl-heterocyclyl-C(O)—.

In some embodiments of the first aspect, the compounds have the Formula (I), wherein D is pyridine substituted with one R³⁸, wherein R³⁸ is (R⁹)(R¹⁰)N—C₁-C₆alkyl-C(O)-heterocyclyl-CH₂—.

In some embodiments of the first aspect, the compounds have the Formula (I), wherein D is pyridine substituted with one R³⁸, wherein R³⁸ is (R⁹)(R¹⁰)N—C(O)—C₁-C₆alkyl-heterocyclyl-CH₂—.

In some embodiments of the first aspect, the compounds have the Formula (I), wherein D is pyridine substituted with one R³⁸, wherein R³⁸ is (R⁹)(R¹⁰)N—C₁-C₆alkyl-C(O)—O—C₁-C₆ alkyl-heterocyclyl-CH₂

In some embodiments of the first aspect, the compounds have the Formula (I), wherein D is pyridine substituted with one R³⁸, wherein R³⁸ is NC—C₁-C₆alkyl-heterocyclyl-CH₂

In some embodiments of the first aspect, the compounds have the Formula (I), wherein D is pyridine substituted with one R³⁸, wherein R³⁸ is F₃C—C₁-C₆alkyl-heterocyclyl-CH₂—.

In some embodiments of the first aspect, the compounds have the Formula (I), wherein D is pyridine substituted with one R³⁸, wherein R³⁸ is N(R⁹)(R¹⁰)N—C₁-C₆alkyl-C(O)—O—C₁-C₆alkyl-C(O)-heterocyclyl-CH₂—.

In some embodiments of the first aspect, the compounds have the Formula (I), wherein D is pyridine substituted with one R³⁸, wherein R³⁸ is (optionally substituted 8- to 10-membered fused heterocyclyl)-C₁-C₆alkyl-.

In some embodiments of the first aspect, the compounds have the Formula (I), wherein D is pyridine substituted with one R³⁸, wherein R³⁸ is (optionally substituted 8- to 10-membered fused heterocyclyl)-C₁-C₆alkyl-, wherein the optional substituent is selected from the group consisting of H, halogen, —N(R⁹)(R¹⁰), nitro, —OH, oxo, C₁-C₆alkyl, —C(O)—C₁-C₆alkyl-OH, Ac, cycloalkyl, heterocyclyl, aryl, heteroaryl, —S(O)₀₋₂—C₁-C₆alkyl, —S(O)₀₋₂-cycloalkyl, —S(O)₀₋₂-heterocyclyl, —S(O)₀₋₂-aryl, —S(O)₀₋₂-heteroaryl, —C(O)H, —C(O)—C₁-C₆alkyl, —C(O)—N(R⁹)(R¹⁰), —C₁-C₆alkyl-OH, —C₁-C₆alkyl-C(O)—OH and)-C₁-C₆alkyl-C(O)—N(R⁹)(R¹⁰), wherein the alkyl, cycloalkyl, heterocyclyl, aryl and heteroaryl groups are themselves optionally substituted, for example with halogen or —C₁-C₆alkyl.

In some embodiments of the first aspect, the compounds have the Formula I, wherein D is imidazole substituted with one R³⁸ and one C₁-C₆alkyl.

In some embodiments of the first aspect, the compounds have the Formula I, wherein D is imidazole substituted with one R³⁸ and one C₁-C₆alkyl, wherein R³⁸ is R³⁷O—(CH₂)₁₋₆—N(A)-(CH₂)₁₋₄—.

In some embodiments of the first aspect, the compounds have the Formula I, wherein D is imidazole substituted with one R³⁸ and one C₁-C₆alkyl, wherein R³⁸ is R³⁷O—(CH₂)₁₋₆—N(A)-(CH₂)₁₋₄—, and A is —C(O)—N(R³⁹)—C₁-C₆alkyl or —C(O)—N(R³⁹)-cycloalkyl.

In some embodiments of the first aspect, the compounds have the Formula I, wherein D is imidazole substituted with one R³⁸ and one C₁-C₆alkyl, wherein R³⁸ is R³⁷O—(CH₂)₂—N(A)-(CH₂)—, and A is —C(O)—N(R³⁹)—C₁-C₆alkyl or —C(O)—N(R³⁹)-cycloalkyl.

In some embodiments of the first aspect, the compounds have the Formula I, wherein D is imidazole substituted with one R³⁸ and one C₁-C₆alkyl, wherein R³⁸ is R³⁷O—(CH₂)₂—N(A)-(CH₂)—, and A is —C(O)—N(R³⁹)—C₁-C₆alkyl.

In some embodiments of the first aspect, the compounds have the Formula I, wherein D is imidazole substituted with one R³⁸, wherein R³⁸ is C₁-C₆alkyl-C(O)—O—C₁-C₆alkyl-C(O)-(5 to 10-membered heterocyclyl)-C₁-C₆alkyl-.

In some embodiments of the first aspect, the compounds have the Formula (I), wherein D is phenyl substituted with one R³⁸.

In some embodiments of the first aspect, the compounds have the Formula (I), wherein D is phenyl substituted with one R³⁸, wherein R³⁸ is R³⁷O—(CH₂)₁₋₆—N(A)-(CH₂)₁₋₄—.

In some embodiments of the first aspect, the compounds have the Formula (I), wherein D is phenyl substituted with one R³⁸, wherein R³⁸ is R³⁷O—(CH₂)₁₋₆—N(A)-(CH₂)₁₋₄—, and A is —C(O)—N(R³⁹)—C₁-C₆alkyl or —C(O)—N(R³⁹)-cycloalkyl.

In some embodiments of the first aspect, the compounds have the Formula (I), wherein D is phenyl substituted with one R³⁸, wherein R³⁸ is R³⁷O—(CH₂)₂—N(A)-(CH₂)—, and A is —C(O)—N(R³⁹)—C₁-C₆alkyl or —C(O)—N(R³⁹)-cycloalkyl.

In some embodiments of the first aspect, the compounds have the Formula (I), wherein D is tetrahydropyridine substituted with one R³⁸.

In some embodiments of the first aspect, the compounds have the Formula (I), wherein D is tetrahydropyridine substituted with one R³⁸, wherein R³⁸ is R³⁷O—C(O)—C₁-C₆alkyl-C(O)— or R³⁷—O—C₁-C₆alkyl-O—C₁-C₆alkyl-C(O)—.

In some embodiments of the first aspect, the compounds have the Formula (I), wherein D is tetrahydropyridine substituted with one R³⁸, wherein R³⁸ is R³⁷O—C(O)—C₁-C₆alkyl-C(O)—.

In some embodiments of the first aspect, the compounds have the Formula (I), wherein D is tetrahydropyridine substituted with one R³⁸, wherein R³⁸ is R³⁷—O—C₁-C₆alkyl-O—C₁-C₆alkyl-C(O)—.

In some embodiments of the first aspect, the compounds have the Formula (I), wherein D is pyrazole substituted with one R³⁸.

In some embodiments of the first aspect, the compounds have the Formula (I), wherein D is pyrazole substituted with one R³⁸, wherein the R³⁸ is cycloalkyl-N(R³⁹)—C(O)—O—C₁-C₆alkyl- or R³⁷O—(CH₂)₁₋₆—N(A)-(CH₂)₁₋₄—.

In some embodiments of the first aspect, the compounds have the Formula (I), wherein D is pyrazole substituted with one R³⁸, wherein R³⁸ is cycloalkyl-N(R³⁹)—C(O)—O—C₁-C₆alkyl- or R³⁷O—(CH₂)₁₋₆—N(A)-(CH₂)₁₋₄—, and A is —C(O)—N(R³⁹)—C₁-C₆alkyl.

In some embodiments of the first aspect, the compounds have the Formula (I), wherein D is pyrazole substituted with one R³⁸, wherein the R³⁸ is cycloalkyl-N(R³⁹)—C(O)—O—C₁-C₆ alkyl-.

In some embodiments of the first aspect, the compounds have the Formula (I), wherein D is pyrazole substituted with one R³⁸, wherein the R³⁸ is R³⁷O—(CH₂)₁₋₆—N(A)-(CH₂)₁₋₄—

In some embodiments of the first aspect, the compounds have the Formula (I), wherein D is pyrazole substituted with one R³⁸, wherein the R³⁸ is R³⁷O—(CH₂)₁₋₆—N(A)-(CH₂)₁₋₄—, and A is —C(O)—N(R³⁹)—C₁-C₆alkyl.

In some embodiments of the first aspect, the compounds have the Formula (I), wherein D is pyrazole substituted with one R³⁸, wherein the R³⁸ is R³⁷O—(CH₂)₂—N(A)-(CH₂)₂—, and A is —C(O)—N(R³⁹)—C₁-C₆alkyl.

In some embodiments of the first aspect, the compounds have the Formula (I), wherein R³⁸ is R³⁷O—(CH₂)₁₋₆—N(A)-(CH₂)₁₋₄—, alternatively R³⁷O—(CH₂)₂—N(A)-(CH₂)₁₋₂—, MeO—(CH₂)₂—N(A)-CH₂— or MeO—(CH₂)₂—N(A)-(CH₂)₂—.

In some embodiments of the first aspect, the compounds have the Formula (I), wherein R³⁸ is C₁-C₆ alkyl-S(O)₂—(CH₂)₂—N(A)-CH₂—, alternatively CH₃—S(O)₂—(CH₂)₂—N(A)-CH₂

In some embodiments of the first aspect, the compounds have the Formula (I), wherein R³⁸ is R³⁷O—(CH₂)ⱼ—[(CH₂)ᵢO]ₓ—(CH₂)ᵢ₁—N(A)-(CH₂)ⱼ₁—, alternatively CH₃—O—[CH₂—CH₂—O]₃—(CH₂)₂—N(A)-CH₂—.

In some embodiments of the first aspect, the compounds have the Formula (I), wherein R³⁸ is R³⁷O—C(O)—C₀-C₆alkyl-heterocyclyl-CH₂—, alternatively R³⁷O—C(O)—C₁-C₆alkyl-heterocyclyl-CH₂—, alternatively HO—C(O)—(CH₂)₂-piperazine-CH₂—, EtO—C(O)-piperidine-CH₂—, EtO—C(O)—CH₂-piperidine-CH₂—, EtO—C(O)—CH₂-piperazine-CH₂—, HO—C(O)-piperidine-CH₂—, HO—C(O)—CH₂-piperidine-CH₂—HO—C(O)—CH₂-piperazine-CH₂—, (CH₃)₃C—O—C(O)-piperazine-CH₂— or HO—C(O)-pyrrolidine-CH₂—.

In some embodiments of the first aspect, the compounds have the Formula (I), wherein R³⁸ is R³⁷O—(CH₂)ⱼ—[(CH₂)ᵢO]ₓ—(CH₂)ᵢ₁—N(R³⁹)—C(O)—, alternatively CH₃—O—[CH₂—CH₂—O]₃—(CH₂)₂—N(A)-C(O)—.

In some embodiments of the first aspect, the compounds have the Formula (I), wherein R³⁸ is R³⁷—O—C(O)—C₁-C₆alkyl-heterocyclyl-C(O)—, alternatively CH₃—CH₂—O—C(O)—(CH₂)₂-piperazine-C(O)— or HO—C(O)—(CH₂)₂-piperazine-C(O)—.

In some embodiments of the first aspect, the compounds have the Formula (I), wherein R³⁸ is HOOC—C₁-C₆alkyl-N(A)-CH₂—, alternatively HOOC—(CH₂)₃—N(A)-CH₂—.

In some embodiments of the first aspect, the compounds have the Formula (I), wherein R³⁸ is (HOOC)(NR⁹R¹⁰)—C₁-C₆alkyl-N(A)-CH₂—, alternatively (HOOC)(NH₂)CH—(CH₂)₄—N(A)-CH₂—.

In some embodiments of the first aspect, the compounds have the Formula (I), wherein R³⁸ is R³⁷O—C(O)—C₁-C₆alkyl-C(O)—, alternatively HO—C(O)—(CH₂)₂—C(O)—.

In some embodiments of the first aspect, the compounds have the Formula (I), wherein R³⁸ is R⁹)(R¹⁰)N—C₁-C₆alkyl-C(O)-heterocyclyl-CH₂—, alternatively

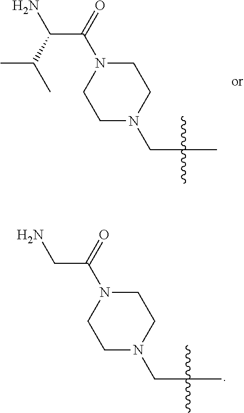 [SMILES: *CN1CCN(C(=O)[C@@H](N)C(C)C)CC1 ; *CN1CCN(C(=O)CN)CC1]

In some embodiments of the first aspect, the compounds have the Formula (I), wherein R³⁸ is cycloalkyl-N(R³⁹)—C(O)—O—C₁-C₆alkyl-, alternatively C₃cycloalkyl-NH—C(O)—O—(CH₂)₂—.

In some embodiments of the first aspect, the compounds have the Formula (I), wherein R³⁸ is R³⁷—O—C₁-C₆alkyl-O—C₁-C₆alkyl-C(O)—, alternatively MeO—(CH₂)₂—O—CH₂—C(O)—.

In some embodiments of the first aspect, the compounds have the Formula (I), wherein

R³⁸ is (R⁹)(R¹⁰)N—C(O)—C₁-C₆alkyl-heterocyclyl-CH₂—, alternatively

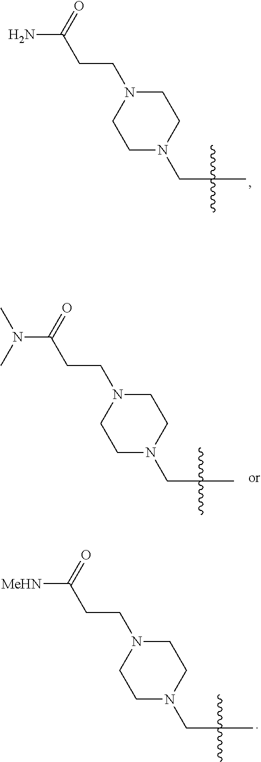 [SMILES: *CN1CCN(CCC(N)=O)CC1 ; *CN1CCN(CCC(=O)N(C)C)CC1 ; *CN1CCN(CCC(=O)NC)CC1]

In some embodiments of the first aspect, the compounds have the Formula (I), wherein R³⁸ is (R⁹)(R¹⁰)N—C₁-C₆alkyl-C(O)—O—C₁-C₆alkyl-heterocyclyl-CH₂—, alternatively

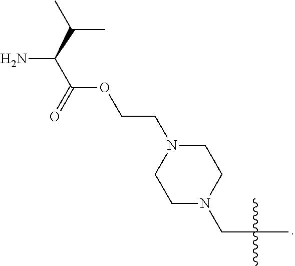 [SMILES: *CN1CCN(CCOC(=O)[C@@H](N)C(C)C)CC1]

In some embodiments of the first aspect, the compounds have the Formula (I), wherein R³⁸ is NC—C₁-C₆alkyl-heterocyclyl-CH₂—, alternatively NC—(CH₂)₂-piperazine-CH₂—.

In some embodiments of the first aspect, the compounds have the Formula (I), wherein R³⁸ is F₃C—C₁-C₆alkyl-heterocyclyl-CH₂—, alternatively F₃C—CH₂-piperazine-CH₂—.

In some embodiments of the first aspect, the compounds have the Formula (I), wherein R³⁸ is (R⁹)(R¹⁰)N—C₁-C₆alkyl-C(O)—O—C₁-C₆alkyl-C(O)-heterocyclyl-CH₂—, alternatively

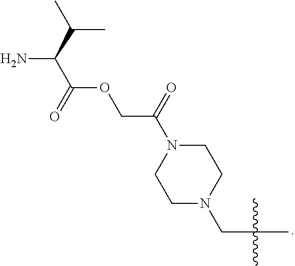 [SMILES: *CN1CCN(C(=O)COC(=O)[C@@H](N)C(C)C)CC1]

In some embodiments of the first aspect, the compounds have the Formula (I), wherein R³⁸ is C₁-C₆alkyl-C(O)—O—C₁-C₆alkyl-C(O)-(5 to 10-membered heterocyclyl)-C₁-C₆alkyl-.

In some embodiments of the first aspect, the compounds have the Formula (I), wherein R³⁸ is C₁-C₆alkyl-C(O)—O—C₁-C₆alkyl-C(O)-(5 to 10-membered heterocyclyl)-C₁-C₆alkyl-, wherein the heterocyclyl is a 6-membered heterocyclyl.

In some embodiments of the first aspect, the compounds have the Formula (I), wherein R³⁸ is C₁-C₆alkyl-C(O)—O—C₁-C₆alkyl-C(O)-(5 to 10-membered heterocyclyl)-C₁-C₆alkyl-, which is

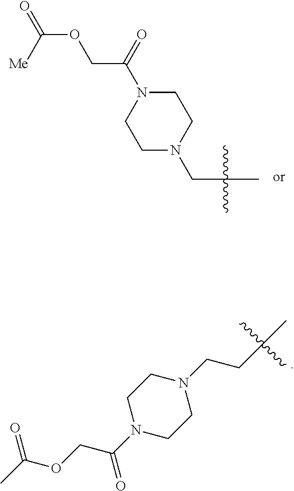 [SMILES: *CN1CCN(C(=O)COC(C)=O)CC1 ; *CCN1CCN(C(=O)COC(C)=O)CC1]

In some embodiments of the first aspect, the compounds have the Formula (I), wherein R³⁸ is (optionally substituted 8- to 10-membered fused heterocyclyl)-C₁-C₆alkyl-.

In some embodiments of the first aspect, the compounds have the Formula (I), wherein R³⁸ is (optionally substituted 8- to 10-membered fused heterocyclyl)-C₁-C₆alkyl, which is

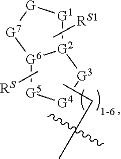

wherein

G is selected from the group consisting of CH₂, O, NH, S, SO and SO₂;

G¹ is selected from the group consisting of CH₂, O, NH, S, SO and SO₂;

G² is CH or N;

G³ is selected from the group consisting of CH₂, O, NH, S, SO and SO₂;

G⁴ is selected from the group consisting of CH₂, O, NH, S, SO and SO₂;

G⁵ is selected from the group consisting of CH₂, O, NH, S, SO and SO₂;

G⁶ is CH or N;

G⁷ is selected from the group consisting of CH₂, O, NH, S, SO and SO₂;

R^{s} is an optional substituent; and

R^{s1} is an optional substituent,

provided that two O atoms are not adjacent to each other.

In some embodiments of the first aspect, the compounds have the Formula (I), wherein R³⁸ is (optionally substituted 8- to 10-membered fused heterocyclyl)-C₁-C₆alkyl, selected from

the group consisting of

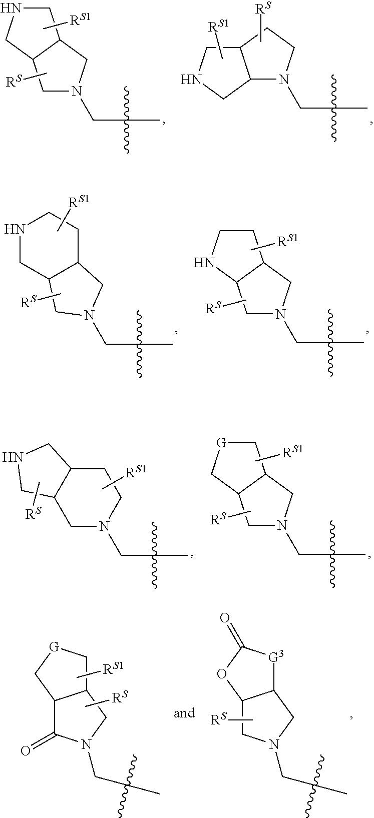

In some embodiments of the first aspect, the compounds have the Formula (I), wherein R³⁸ is (optionally substituted 8- to 10-membered fused heterocyclyl)-C₁-C₆alkyl, selected from the group consisting of

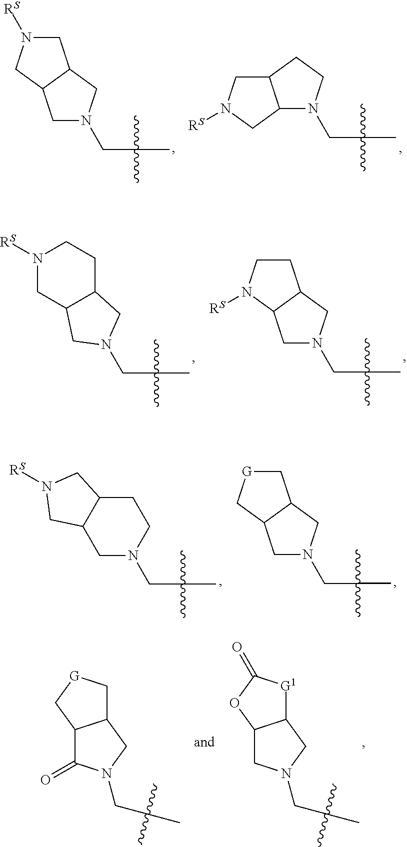

wherein G is selected from the group consisting of CH₂, O, NH, S, SO and SO₂; G¹ is selected from the group consisting of CH₂, O, NH, S, SO and SO₂; and R^{s} is an optional substituent.

In some embodiments of the first aspect, the compounds have the Formula (I), wherein R^{s} is selected from the group consisting of H, halogen, —N(R⁹)(R¹⁰), nitro, —OH, oxo, C₁-C₆alkyl, —C(O)—C₁-C₆alkyl-OH, Ac, cycloalkyl, heterocyclyl, aryl, heteroaryl, —S(O)₀₋₂—C₁-C₆alkyl, —S(O)₀₋₂-cycloalkyl, —S(O)₀₋₂-heterocyclyl, —S(O)₀₋₂-aryl, —S(O)₀₋₂-heteroaryl, —C(O)H, —C(O)—C₁-C₆alkyl, —C(O)—N(R⁹)(R¹⁰), —C₁-C₆alkyl-OH, —C₁-C₆alkyl-C(O)—OH, —C₁-C₆alkyl-C(O)—N(R⁹)(R¹⁰), wherein the alkyl, cycloalkyl, heterocyclyl, aryl and heteroaryl groups are themselves optionally substituted, for example with halogen or —C₁-C₆alkyl.

In some embodiments of the first aspect, the compounds have the Formula (I), wherein R^{s1} is selected from the group consisting of H, halogen, —N(R⁹)(R¹⁰), nitro, —OH, oxo, C₁-C₆alkyl, —C(O)—C₁-C₆alkyl-OH, Ac, cycloalkyl, heterocyclyl, aryl, heteroaryl, —S(O)₀₋₂—C₁-C₆alkyl, —S(O)₀₋₂-cycloalkyl, —S(O)₀₋₂-heterocyclyl, —S(O)₀₋₂-aryl, —S(O)₀₋₂-heteroaryl, —C(O)H, —C(O)—C₁-C₆alkyl, —C(O)—N(R⁹)(R¹⁰), —C₁-C₆alkyl-OH, —C₁-C₆alkyl-C(O)—OH, —C₁-C₆alkyl-C(O)—N(R⁹)(R¹⁰), wherein the alkyl, cycloalkyl, heterocyclyl, aryl and heteroaryl groups are themselves optionally substituted, for example with halogen or —C₁-C₆alkyl.

In some embodiments of the first aspect, the compounds have the Formula (I), wherein R³⁸ is (optionally substituted 8- to 10-membered fused heterocyclyl)-C₁-C₆alkyl-, wherein the optional substituent is selected from the group consisting of H, halogen, —N(R⁹)(R¹⁰), nitro, —OH, oxo, C₁-C₆ alkyl, —C(O)—C₁-C₆alkyl-OH, Ac, cycloalkyl, heterocyclyl, aryl, heteroaryl, —S(O)₀₋₂—C₁-C₆alkyl, —S(O)₀₋₂-cycloalkyl, —S(O)₀₋₂-heterocyclyl, —S(O)₀₋₂-aryl, —S(O)₀₋₂-heteroaryl, —C(O)H, —C(O)—C₁-C₆alkyl, —C(O)—N(R⁹)(R¹⁰), —C₁-C₆alkyl-OH, —C₁-C₆alkyl-C(O)—OH and —C₁-C₆alkyl-C(O)—N(R⁹)(R¹⁰), wherein the alkyl, cycloalkyl, heterocyclyl, aryl and heteroaryl groups are themselves optionally substituted, for example with halogen or —C₁-C₆alkyl.

In some embodiments of the first aspect, the compounds have the Formula (I), wherein A is —C(O)—C₁-C₆alkyl-N(R³⁹)—C(O)—C₁-C₆alkyl-N(R⁹)(R¹⁰), alternatively —C(O)—CH₂—NH—C(O)—CH(NH₂)—CH(CH₃)₂, —C(O)—CH₂—NH—C(O)—CH₂—NH₂ or —C(O)—CH[CH(CH₃)₂]—NH—C(O)—CH₂—NH₂).

In some embodiments of the first aspect, the compounds have the Formula (I), wherein A is —C(O)—N(R³⁹)—C₁-C₆alkyl, alternatively —C(O)—NH—CH₂—CH₃, —C(O)—NH—CH₃, —C(O)—NH—CH(CH₃)₂, —C(O)—NH—CH(CH₃)₂ or —C(O)—N(CH₃)₂.

In some embodiments of the first aspect, the compounds have the Formula (I), wherein A is —C(═NR³⁷)—C₁-C₆alkyl, alternatively —C(═NH)H.

In some embodiments of the first aspect, the compounds have the Formula (I), wherein A is —C(O)—(CH₂)ₙ—S(O)₂—C₁-C₆alkyl, alternatively —C(O)—CH₂—S(O)₂-Me.

In some embodiments of the first aspect, the compounds have the Formula (I), wherein A is —C(O)—N(R³⁹)-cycloalkyl, alternatively —C(O)—NH-cyclopentyl or —C(O)—NH—C₃ cycloalkyl.

In some embodiments of the first aspect, the compounds have the Formula (I), wherein A is —C(O)—N(R⁹)(R¹⁰), alternatively —C(O)—NH₂.

In some embodiments of the first aspect, the compounds have the Formula (I), wherein A is (R³⁷O)(R^{37a}O)P(O)O—C₁-C₆alkyl-C(O)—, alternatively (HO)₂P(O)O—CH₂—C(O)—.

In some embodiments of the first aspect, the compounds have the Formula (I), wherein M is a structure selected from the group consisting of

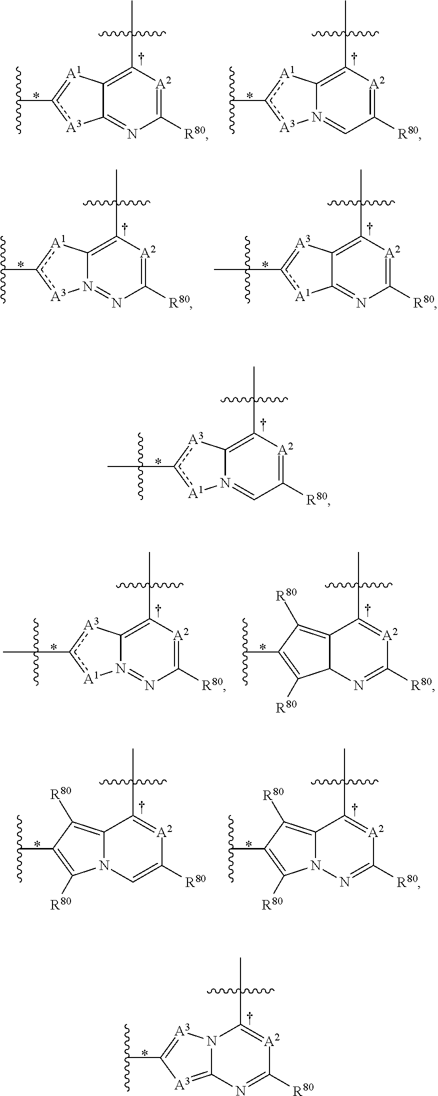

wherein
* represents the point of attachment to D;
† represents the point of attachment to Z;
A¹ is selected from the group consisting of CH, —O—, —S—, —N(H)—, —N(C₁-C₆ alkyl)-, —N—(Y-aryl)-, —N-OMe, —NCH₂OMe and N-Bn;
Y is a bond or —(C(R^{x})(H))ₜ—, wherein t is an integer from 1 to 6; and
R^{x} at each occurrence is independently selected from the group consisting of H and C₁-C₆ alkyl, wherein the C₁-C₆ alkyl is optionally substituted;
A² is selected from the group consisting of N and CR, wherein R is selected from the group consisting of —H, halogen, —CN, C₁-C₆ alkyl, C₂-C₆ alkenyl, C₂-C₆ alkynyl, —COOH and —C(O)Oalkyl, wherein the C₁-C₆ alkyl, C₂-C₆ alkenyl, C₂-C₆ alkynyl and —C(O)Oalkyl are optionally substituted;
each A³ is independently selected from the group consisting of CH and N;
each R⁸⁰ is independently selected from the group consisting of H, halogen, NO₂, cyano, OR⁸³, N(R⁸³)₂, CO₂R⁸³, C(O)N(R⁸³)₂, SO₂R⁸³, SO₂N(R⁸³)₂, NR⁸³SO₂R⁸³, NR⁸³C(O)R⁸³, NR⁸³CO₂R⁸³, —CO(CH₂)₁R⁸³, —CONH(CH₂)₁R⁸³, alkylaminoalkyl, alkylaminoalkynyl, C₁-C₆alkyl, substituted C₁-C₆alkyl, C₃-C₇cycloalkyl, substituted C₃-C₇cycloalkyl, alkenyl, substituted alkenyl, alkynyl, substituted alkynyl, hydroxyalkyl, aryl, substituted aryl, heteroaryl, substituted heteroaryl, arylalkyl, substituted arylalkyl, heterocycloalkyl, and substituted heterocycloalkyl; and
each R⁸³ is independently selected from the group consisting of H, alkyl, substituted alkyl, cycloalkyl, substituted cycloalkyl, aryl, substituted aryl, arylalkyl, substituted arylalkyl, heteroaryl, substituted heteroaryl, heterocycloalkyl, and substituted heterocycloalkyl; or
two R⁸³ taken together with the N atom to which they are attached form a heterocyclic ring.

In some embodiments of the first aspect, the compounds have the Formula (I), wherein M is a structure selected from the group consisting of

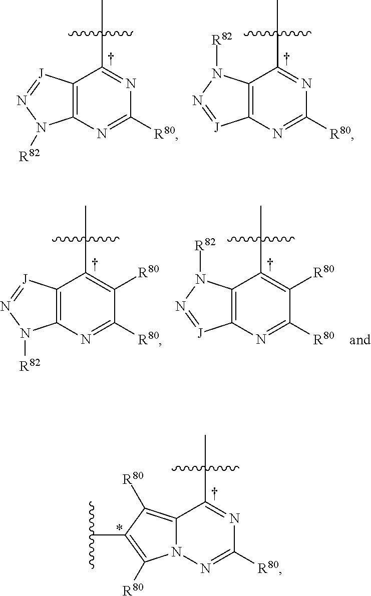

wherein

### J is CR⁸⁰ or N;

R⁸² is selected from the group consisting of H, C₁-C₆alkyl or substituted C₁-C₆alkyl, —Y-(aryl), —Y-(heteroaryl), -alkoxy and —CH₂OMe;
wherein *, †, R⁸⁰ and Y are as defined above.

In some embodiments of the first aspect, the compounds have the Formula (I), wherein M is a structure selected from the group consisting of

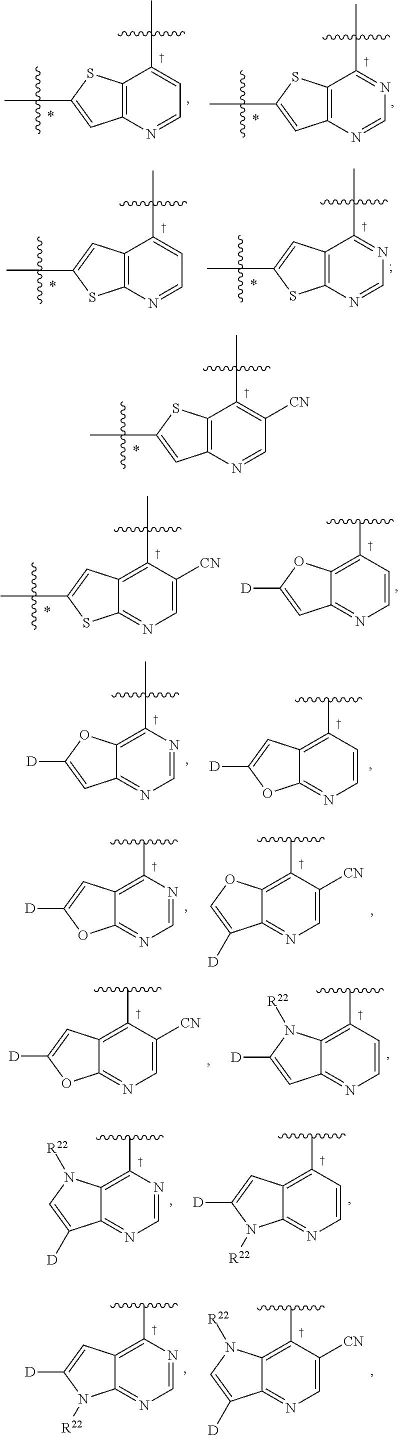

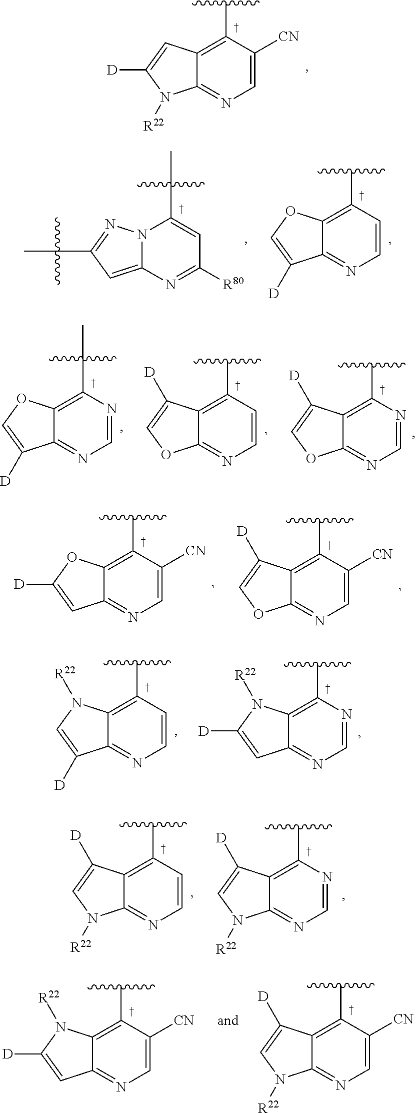

wherein
† is as defined above; and
R²² is selected from the group consisting of —H, —C₁-C₆alkyl, —Y-aryl, alkoxy, —CH₂—O-Me and -Bn.

In some embodiments of the first aspect, the compounds have the Formula (I), wherein M is

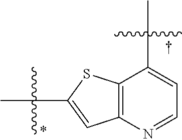 [SMILES: *c1cc2nccc(*)c2s1]

In some embodiments of the first aspect, the compounds have the Formula (I), wherein Z is O.

In some embodiments of the first aspect, the compounds have the Formula (I), wherein Ar is selected from the group consisting of

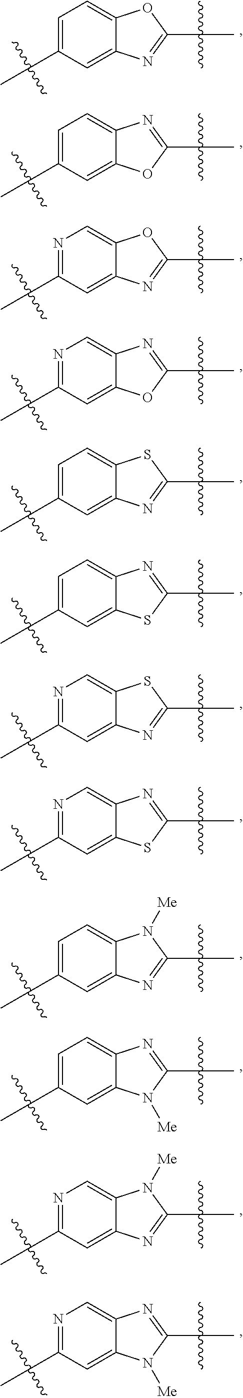

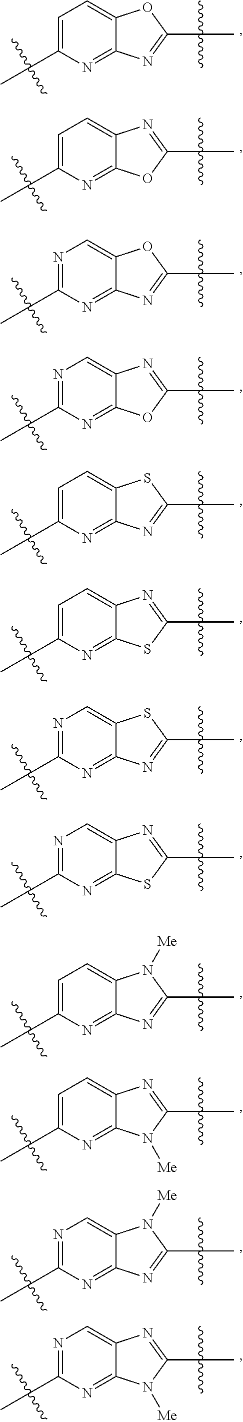

In some embodiments of the first aspect, the compounds have the Formula (I), wherein Ar is selected from the group consisting of

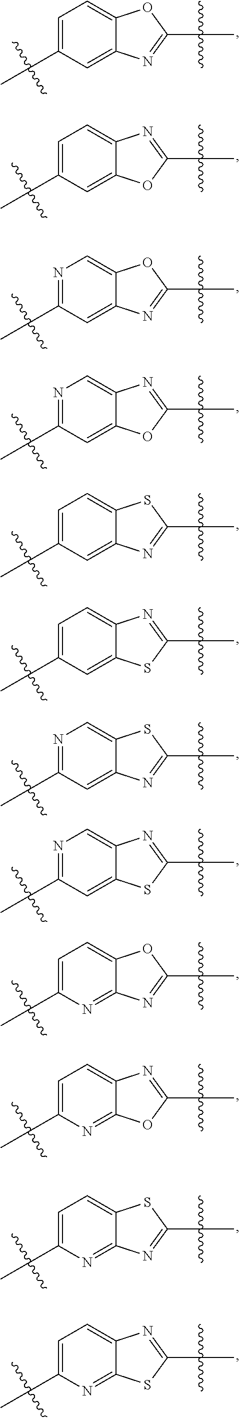

In some embodiments of the first aspect, the compounds have the Formula (I), wherein G is selected from the group consisting of:

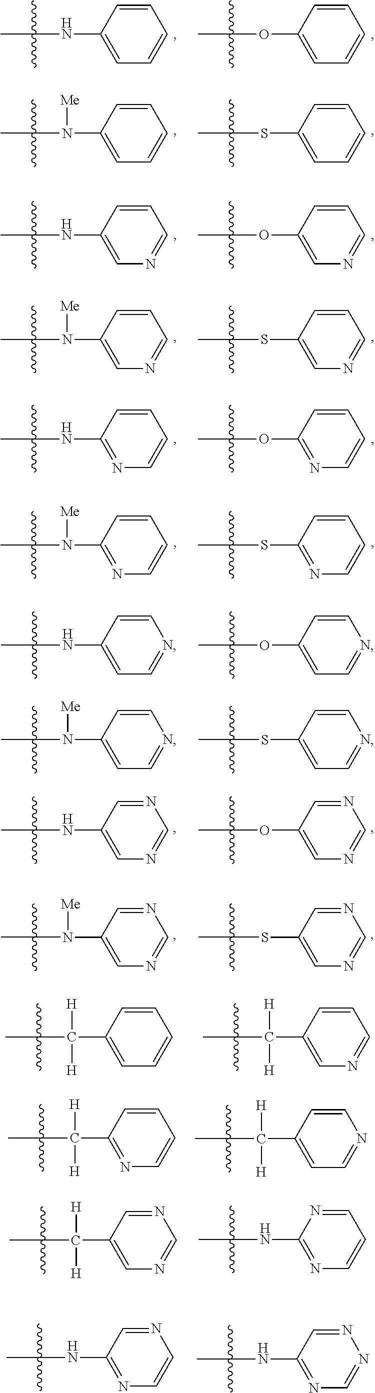

In some embodiments of the first aspect, the compounds have the Formula (I), wherein G is selected from the group consisting of:

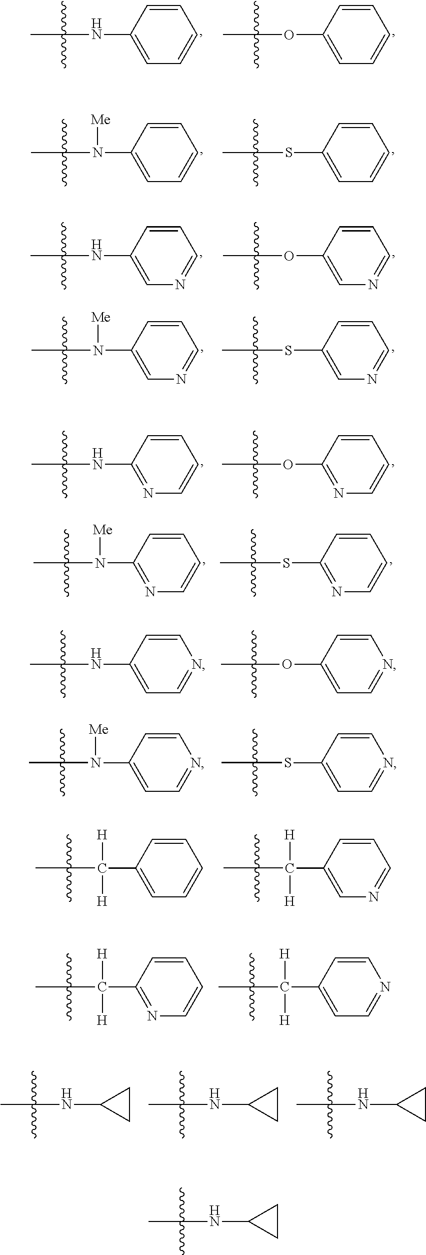

In some embodiments of the first aspect, the compounds are selected from the group consisting of

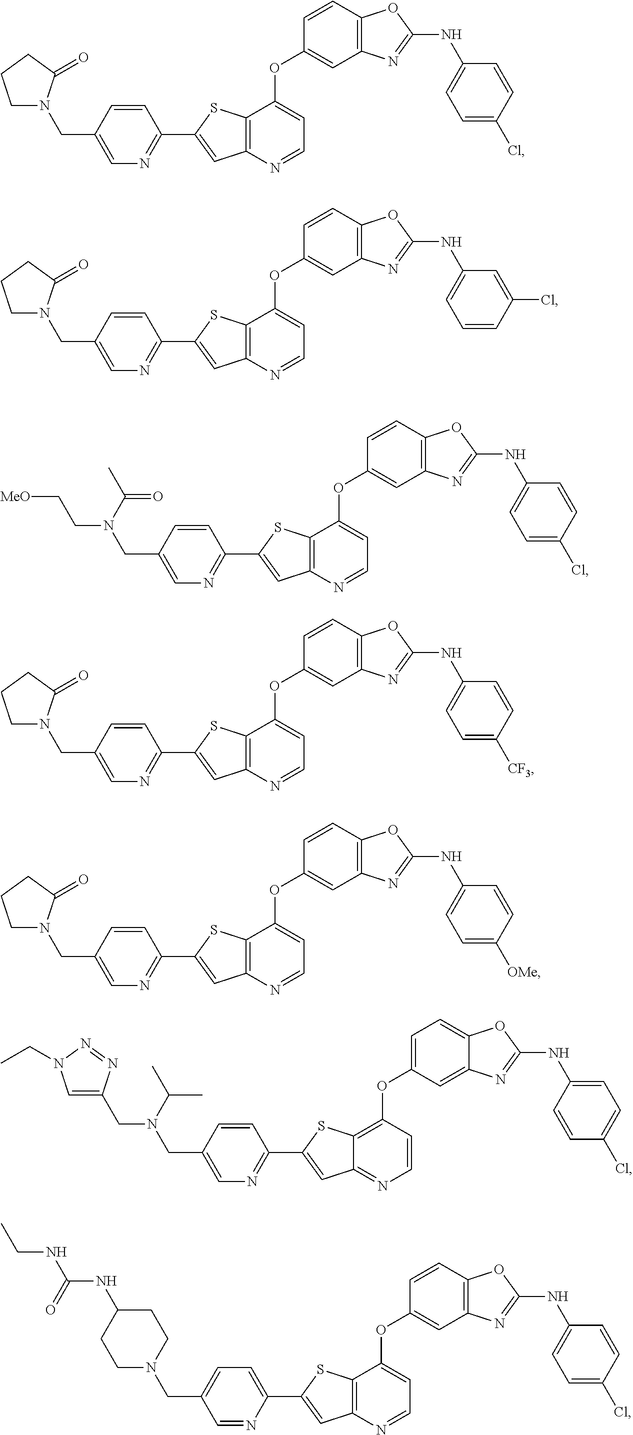

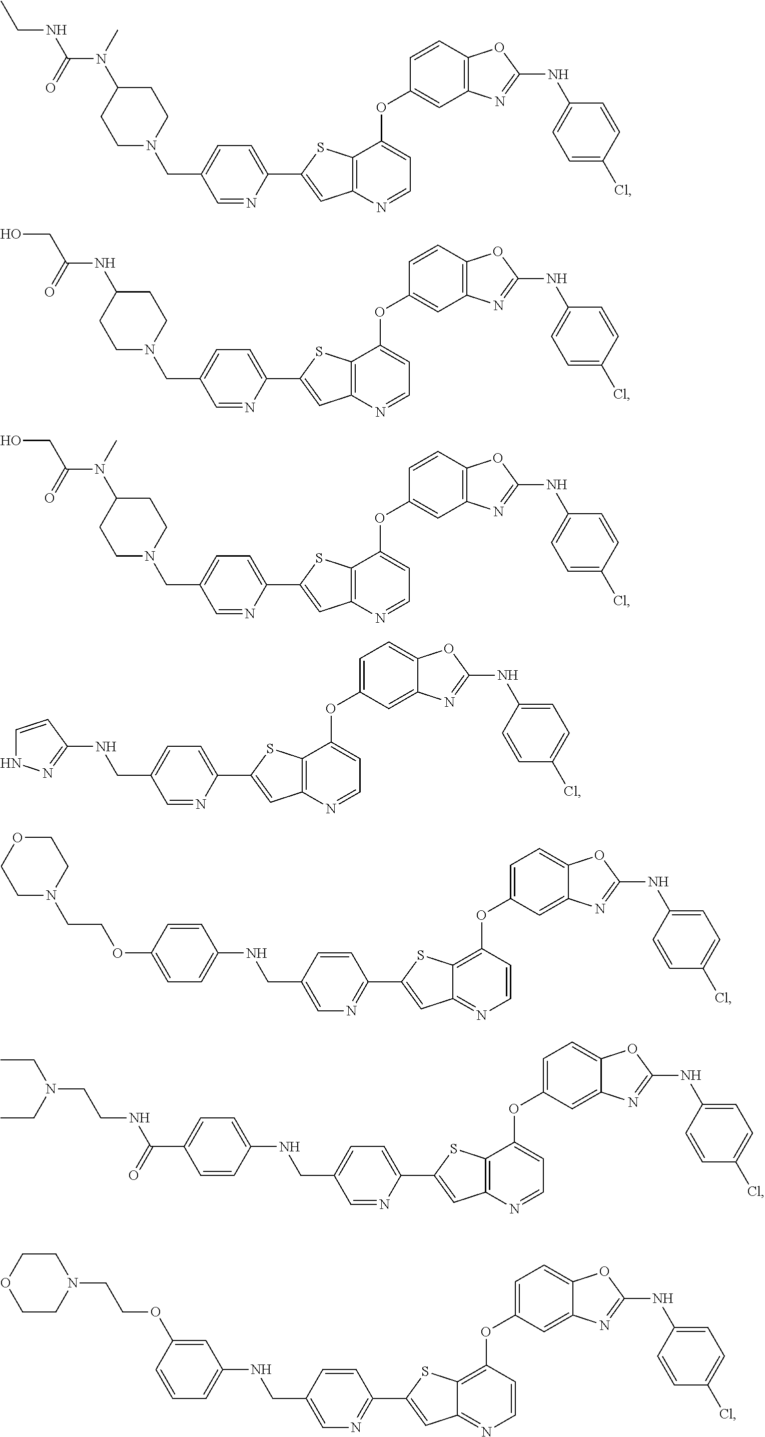

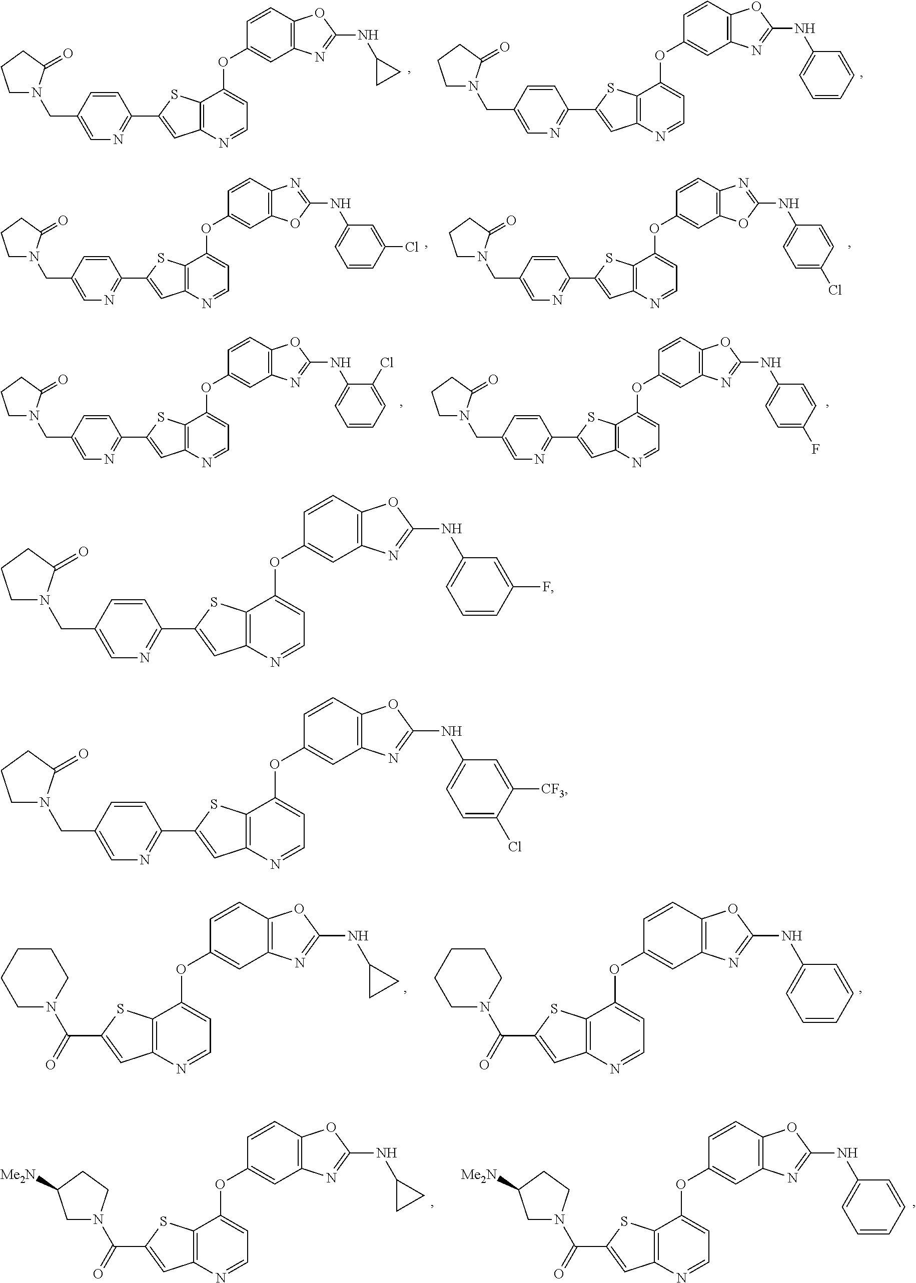

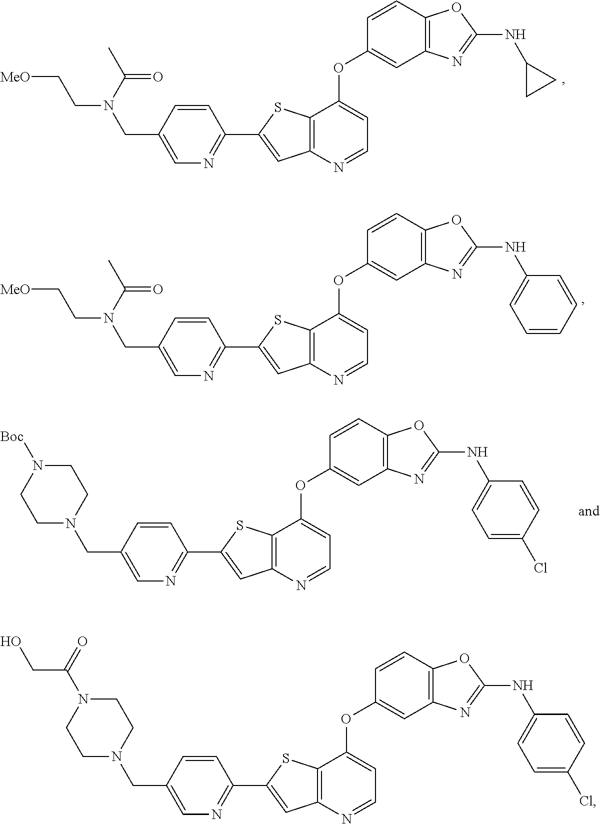

including N-oxides, hydrates, solvates, tautomers, pharmaceutically acceptable salts, prodrugs and complexes thereof, and racemic and scalemic mixtures, diastereomers and enantiomers thereof.

In one embodiment of the first aspect, the compound is

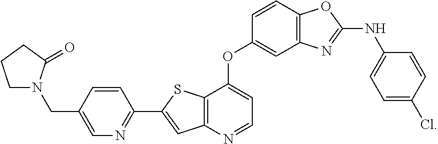 [SMILES: O=C1CCCN1Cc1ccc(-c2cc3nccc(Oc4ccc5oc(Nc6ccc(Cl)cc6)nc5c4)c3s2)nc1]

In one embodiment of the first aspect, the compound is

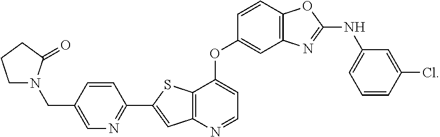 [SMILES: O=C1CCCN1Cc1ccc(-c2cc3nccc(Oc4ccc5oc(Nc6cccc(Cl)c6)nc5c4)c3s2)nc1]

In one embodiment of the first aspect, the compound is

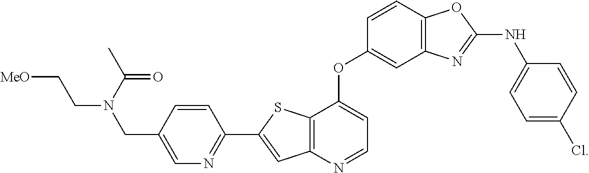 [SMILES: COCCN(Cc1ccc(-c2cc3nccc(Oc4ccc5oc(Nc6ccc(Cl)cc6)nc5c4)c3s2)nc1)C(C)=O]

In one embodiment of the first aspect, the compound is

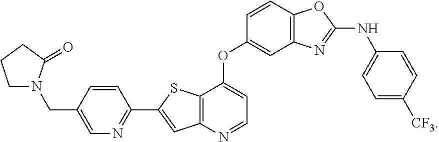 [SMILES: O=C1CCCN1Cc1ccc(-c2cc3nccc(Oc4ccc5oc(Nc6ccc(C(F)(F)F)cc6)nc5c4)c3s2)nc1]

In one embodiment of the first aspect, the compound is

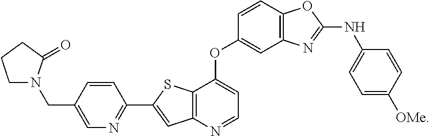 [SMILES: COc1ccc(Nc2nc3cc(Oc4ccnc5cc(-c6ccc(CN7CCCC7=O)cn6)sc45)ccc3o2)cc1]

In one embodiment of the first aspect, the compound is

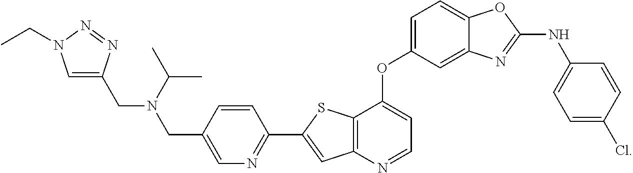 [SMILES: CCn1cc(CN(Cc2ccc(-c3cc4nccc(Oc5ccc6oc(Nc7ccc(Cl)cc7)nc6c5)c4s3)nc2)C(C)C)nn1]

In one embodiment of the first aspect, the compound is

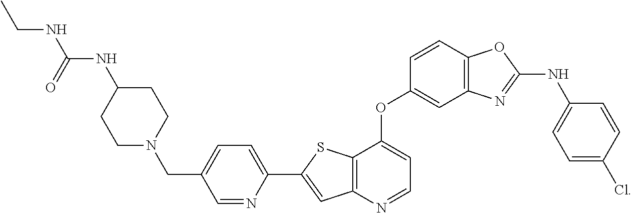 [SMILES: CCNC(=O)NC1CCN(Cc2ccc(-c3cc4nccc(Oc5ccc6oc(Nc7ccc(Cl)cc7)nc6c5)c4s3)nc2)CC1]

In one embodiment of the first aspect, the compound is

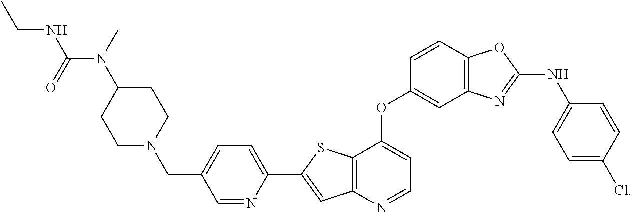 [SMILES: CCNC(=O)N(C)C1CCN(Cc2ccc(-c3cc4nccc(Oc5ccc6oc(Nc7ccc(Cl)cc7)nc6c5)c4s3)nc2)CC1]

In one embodiment of the first aspect, the compound is

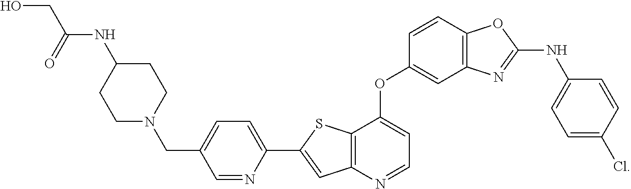 [SMILES: O=C(CO)NC1CCN(Cc2ccc(-c3cc4nccc(Oc5ccc6oc(Nc7ccc(Cl)cc7)nc6c5)c4s3)nc2)CC1]

In one embodiment of the first aspect, the compound is

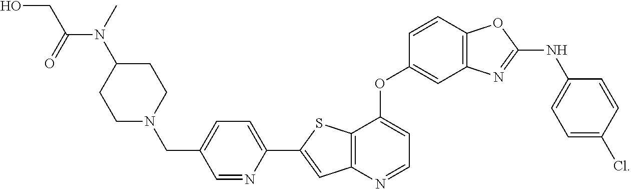 [SMILES: CN(C(=O)CO)C1CCN(Cc2ccc(-c3cc4nccc(Oc5ccc6oc(Nc7ccc(Cl)cc7)nc6c5)c4s3)nc2)CC1]

Compounds of above formulas may generally be prepared according to the following Schemes. Tautomers and solvates (e.g., hydrates) of the compounds of above formulas are also within the scope of the present invention. Methods of solvation are generally known in the art. Accordingly, the compounds of the present invention may be in the free, hydrate or salt form, and may be obtained by methods exemplified by the following schemes below.

The following examples and preparations describe the manner and process of making and using the invention and are illustrative rather than limiting. It should be understood that there may be other embodiments which fall within the spirit and scope of the invention as defined by the claims appended hereto.

Compounds according to the invention include but are not limited to those described in the examples below. Compounds were named using Chemdraw Ultra (versions 10.0, 10.0.4 or version 8.0.3), which are available through Cambridgesoft (www.Cambridgesoft.com, 100 Cambridge Park Drive, Cambridge, Mass. 02140, or were derived therefrom.

The data presented herein demonstrate the inhibitory effects of the kinase inhibitors of the invention. These data lead one to reasonably expect that the compounds of the invention are useful not only for inhibition of kinase activity, protein tyrosine kinase activity, or embodiments thereof, such as, VEGF receptor signaling, but also as therapeutic agents for the treatment of proliferative diseases, including cancer and tumor growth and ophthalmic diseases, disorders and conditions.

### Synthetic Schemes and Experimental Procedures

The compounds of the invention can be prepared according to the reaction schemes or the examples illustrated below utilizing methods known to one of ordinary skill in the art. These schemes serve to exemplify some procedures that can be used to make the compounds of the invention. One skilled in the art will recognize that other general synthetic procedures may be used. The compounds of the invention can be prepared from starting components that are commercially available. Any kind of substitutions can be made to the starting components to obtain the compounds of the invention according to procedures that are well known to those skilled in the art.

All reagents and solvents were obtained from commercial sources and used as received. ¹H-NMR spectra were recorded on a Mercury Plus Varian 400 MHz instrument in the solvents indicated. Low resolution mass-spectra (LRMS) were acquired on an Agilent MSD instrument. Analytical HPLC was performed on an Agilent 1100 instrument using Zorbax 3 μm, XDB-C8, 2.1×50 mm column; eluting with methanol/water containing 0.1% formic acid, with a gradient 5-95% methanol in 15 minutes. Automated column chromatography was performed on a Biotage SP1 or Biotage SP4 instruments using Biotage® SNAP, SiliaSep™ or SiliaFlash® cartridges. Flash column chromatography was performed using silica gel (cartriges SiliaFlash F60, 40-63 μM, pore size 60 Å, SiliCycle®).

Alternatively ¹H-NMR spectra were recorded on a JEOL AL300 300 MHz instrument in the solvents indicated. Low resolution mass-spectra (LRMS) were acquired on an Applied Biosystems/MDS Sciex 4000 QTRAP® instrument. Analytical HPLC was performed on a Shimazu SLC-10Avp machine; column Cadenza 5CD-C18, eluent water containing 0.1% TFA with a gradient of 5-95% MeCN over 15 minutes. Automated column chromatography was performed on a Yamazen Parallel Frac FR-260 apparatus (cartridges HI-FLASH™ COLUMN packed either with silicagel 40 μM or amino silicagel 40 μM)

### PARTICULAR EXAMPLES

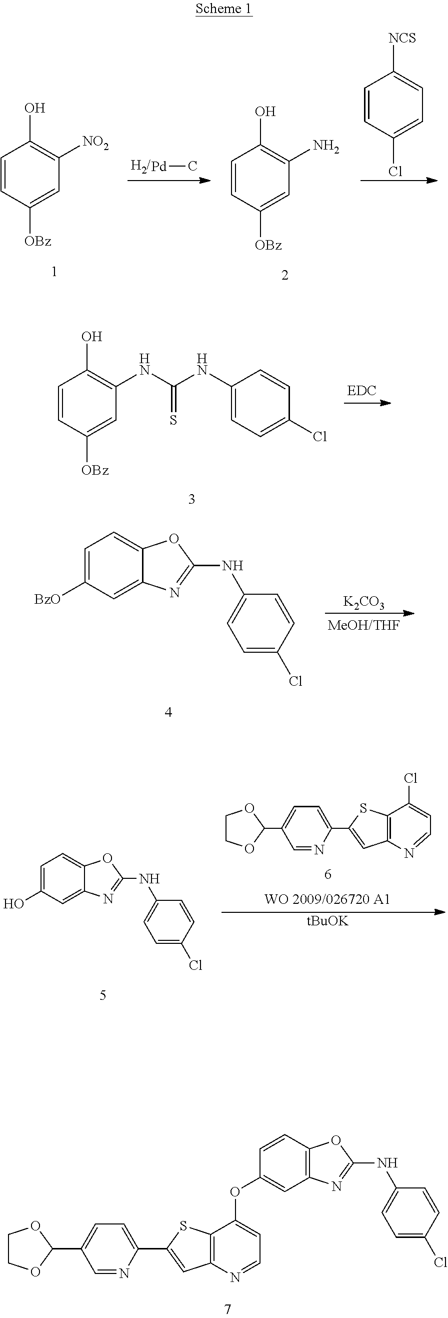

### 5-(2-(5-(1,3-Dioxolan-2-yl)pyridin-2-yl)thieno[3,2-b]pyridin-7-yloxy)-N-(4-chlorophenyl)benzo[d]oxazol-2-amine (7)

### Step 1. 3-Amino-4-hydroxyphenyl benzoate (2)

To a solution of nitrophenol 1 (28.1 g, 0.108 mol, R. Giméneza, et al., Helv.Chim.Acta, 2006, 89, pp 304-319) in EtOAc (300 mL) was added 10% Pd—C (2.8 g) and the reaction mixture was stirred under an atmosphere of H₂ for 5 hours. The mixture was filtered through celite and then concentrated to afford the title compound 2 (24.7 g, quant.) as a beige solid. MS (m/z): 230.09 (M+H).

### Step 2. 3-(3-(4-Chlorophenyl)thioureido)-4-hydroxyphenyl benzoate (3)

To a solution of 2 (18 g, 78.5 mmol) in MeOH (360 mL) was added 4-chlorophenyl isothiocyanate (20 g, 116.7 mmol) at RT and the reaction mixture was stirred overnight. The mixture was concentrated and the residue was triturated with Et₂O to afford the title compound 3 (27.7 g, 69.4 mmol, 88% yield) as a white solid. ¹H NMR (400 MHz, DMSO-d6) δ (ppm): 10.21 (s, 1H), 10.11 (s, 1H), 9.30 (s, 1H), 8.14-8.10 (m, 2H), 8.04 (d, J=2.4 Hz, 1H), 7.77-7.71 (m, 1H), 7.63-7.56 (m, 4H), 7.42-7.38 (m, 2H), 6.96-6.89 (m, 2H). MS (m/z): 399.18 (M+H).

### Step 3. 2-(4-Chlorophenylamino)benzo[d]oxazol-5-yl benzoate (4)

A suspension of 3 (27.7 g, 69.4 mmol) was heated to reflux; EDC (16 g, 83.3 mmol) was added and the reaction mixture was heated for a further 10 min. The mixture was cooled to RT and diluted with EtOAc and water. The resultant precipitate was collected by filtration, rinsed with water, EtOAc and dried to afford the title compound 4 (20.3 g, 55.6 mmol, 80% yield) as a white solid. ¹H NMR (400 MHz, DMSO-d6) δ (ppm): 10.98 (s, 1H), 8.24-8.20 (m, 2H), 7.88-7.80 (m, 3H), 7.72-7.65 (m, 2H), 7.64 (d, J=8.4 Hz, 1H), 7.55-7.49 (m, 2H), 7.47 (d, J=2.4 Hz, 1H), 7.11 (dd, J=8.4, 2.4 Hz, 1H). MS (m/z): 365.20 (M+H).

### Step 4. 2-(4-Chlorophenylamino)benzo[d]oxazol-5-ol (5)

To a suspension of 4 (19.0 g, 52.0 mmol) in MeOH/THF (290/290 mL) was added K₂CO₃ (7.2 g, 52.0 mmol) at RT and the mixture was stirred for 2 hour. To the mixture was added water and the mixture was concentrated to remove MeOH and THF. The resultant precipitate was collected by filtration, rinsed with water and dried to afford the title compound 5 (12.0 g, 46.0 mmol, 89% yield) as a grey solid. ¹H NMR (400 MHz, DMSO-d6) δ (ppm): 10.67 (s, 1H), 9.25 (s, 1H), 7.78-7.72 (m, 2H), 7.45-7.40 (m, 2H), 7.25 (d, J=8.4 Hz, 1H), 6.81 (d, J=2.4 Hz, 1H), 6.53 (dd, J=8.4, 2.4 Hz, 1H). MS (m/z): 261.09 (M+H).

### Step 5. 5-(2-(5-(1,3-Dioxolan-2-yl)pyridin-2-yl)thieno[3,2-b]pyridin-7-yloxy)-N-(4-chlorophenyl)benzo[d]oxazol-2-amine (7)

To a suspension of 5 (0.68 g, 2.63 mmol) and chlorothienopyridine 6 (0.42 g, 1.32 mmol, WO 2009/026720 A1) in DMSO (10 mL) was added tert-BuOK (0.59 g, 5.27 mmol) at RT and the mixture was heated to 90° C. for 1 hour. The reaction mixture was poured into saturated aqueous solution of NH₄Cl to form a precipitate that was collected by filtration, rinsed with water, dried and purified by Biotage (MeOH/EtOAc:0/100-10/90), to afford the title compound 7 (0.29 g, 0.53 mmol, 40% yield) as a brown solid. ¹H NMR (400 MHz, DMSO-d6) δ (ppm): 10.98 (s, 1H), 8.69 (d, J=2.4 Hz, 1H), 8.52 (d, J=5.2 Hz, 1H), 8.40 (s, 1H), 8.32 (dd, J=8.4, 0.4 Hz, 1H), 7.98 (dd, J=8.4, 2.0 Hz, 1H), 7.82-7.76 (m, 2H), 7.64 (d, J=8.4 Hz, 1H), 7.48-7.42 (m, 3H), 7.09 (dd, J=8.8, 2.4 Hz, 1H), 5.89 (s, 1H). MS (m/z): 543.40 (M+H).

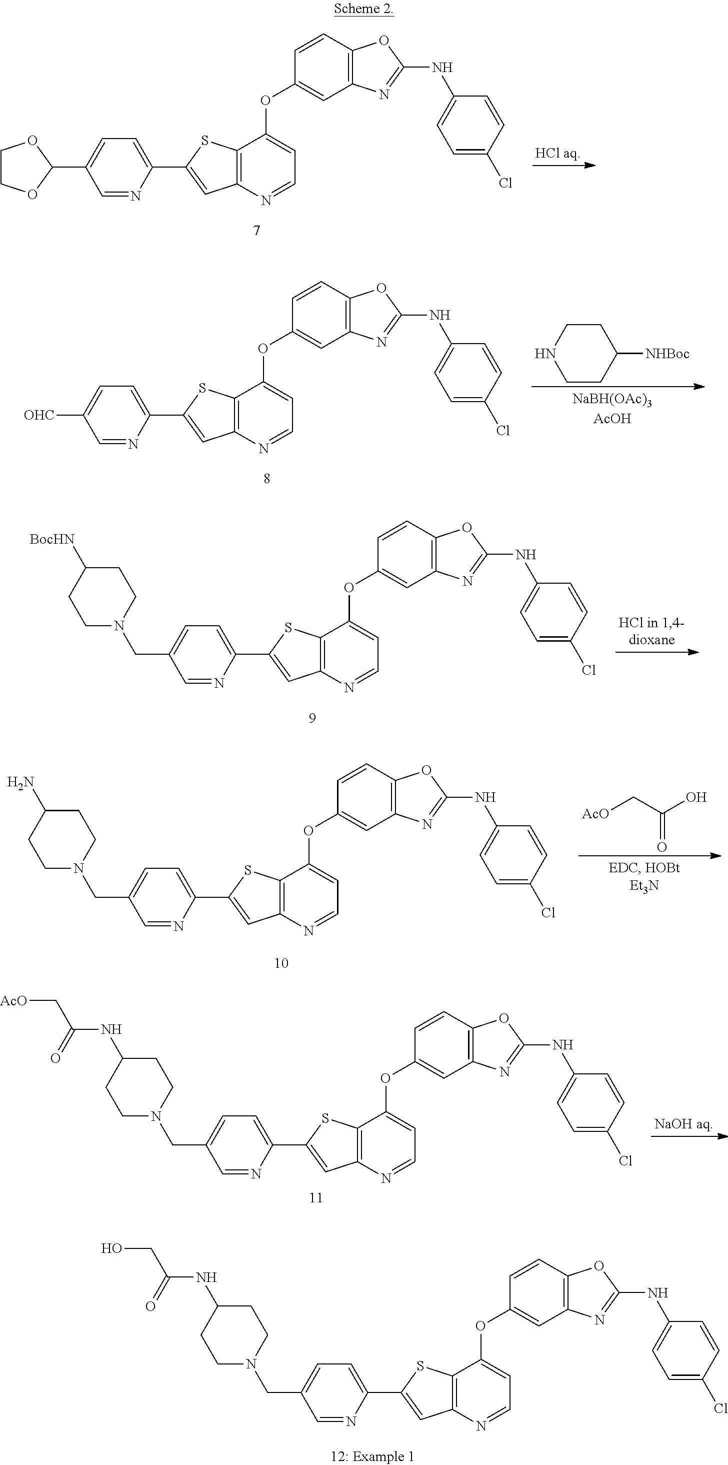

### Example 1

### N-(1-((6-(7-(2-(4-Chlorophenylamino)benzo[d]oxazol-5-yloxy)thieno[3,2-b]pyridin-2-yl)pyridin-3-yl)methyl)piperidin-4-yl)-2-hydroxyacetamide (12, example 1)

### Step 1. 6-(7-(2-(4-Chlorophenylamino)benzo[d]oxazol-5-yloxy)thieno[3,2-b]pyridin-2-yl)nicotinaldehyde (8)

To a suspension of 7 (2.69 g, 4.95 mmol) in THF (60 mL) was added aqueous 4N HCl solution (12 mL, 48 mmol) at RT and the reaction mixture was heated at 50° C. for 3 hours. The mixture was concentrated, diluted with water, basified with a saturated aqueous solution of NaHCO₃ and stirred at RT for 1 hour. The precipitate was collected by filtration, dried and triturated with EtOAc to afford the title compound 8 (2.28 g, 4.57 mmol, 92%) as a brown solid. ¹H NMR (400 MHz, DMSO-d6) δ (ppm): 10.99 (s, 1H), 10.13 (s, 1H), 9.13 (dd, J=2.4, 0.8 Hz, 1H), 8.58 (s, 1H), 8.56 (d, J=5.6 Hz, 1H), 8.51 (d, J=8.0 Hz, 1H), 8.38 (dd, J=8.0, 2.4 Hz, 1H), 7.83-7.76 (m, 2H), 7.64 (d, J=8.8 Hz, 1H), 7.50-7.41 (m, 3H), 7.10 (dd, J=8.4, 2.4 Hz, 1H), 6.70 (d, J=5.6 Hz, 1H). MS (m/z): 499.31 (M+H).

### Step 2. tert-Butyl 1-((6-(7-(2-(4-chlorophenylamino)benzo[d]oxazol-5-yloxy)thieno[3,2-b]pyridin-2-yl)pyridin-3-yl)methyl)piperidin-4-ylcarbamate (9)

To a solution of 8 (0.25 g, 0.49 mmol) in NMP (3 mL) were added acetic acid (141 μL, 2.45 mmol) and 4-N-Boc-aminopiperidine (0.300 g, 1.48 mmol) at RT and the reaction mixture was stirred for 1 hour, NaBH(OAc)₃ (0.31 g, 1.48 mmol) was added and the stirring was continued overnight. The reaction mixture was poured into a saturated aqueous solution of NaHCO₃ to form a precipitate that was collected by filtration, rinsed with water, dried and purified by Biotage (MeOH/DCM: 0/100-20/80), to afford the title compound 9 (0.12 g, 0.181 mmol, 37% yield) as a beige solid. ¹H NMR (400 MHz, DMSO-d6) δ (ppm): 10.99 (s, 1H), 8.52 (brs, 1H), 8.51 (d, J=5.6 Hz, 1H), 8.32 (s, 1H), 8.24 (d, J=8.4 Hz, 1H), 7.84 (dd, J=8.4, 2.0 Hz, 1H), 7.82-7.77 (m, 2H), 7.63 (d, J=8.4 Hz, 1H), 7.49-7.42 (m, 3H), 7.08 (dd, J=8.4, 2.4 Hz, 1H), 6.80 (d, J=7.6 Hz, 1H), 6.65 (d, J=5.2 Hz, 1H), 3.51 (s, 2H), 3.27-3.16 (m, 1H), 2.80-2.72 (m, 2H), 2.05-1.94 (m, 2H), 1.72-1.64 (m, 2H), 1.44-1.32 (m, 2H), 1.37 (s, 9H). MS (m/z): 683.58 (M+H).

### Step 3. 5-(2-(5-((4-Aminopiperidin-1-yl)methyl)pyridin-2-yl)thieno[3,2-b]pyridin-7-yloxy)-N-(4-chlorophenyl)benzo[d]oxazol-2-amine (10)

To a suspension of 9 (0.12 g, 0.181 mmol) in DCM (5 mL) was added 4M HCl in 1,4-dioxane solution (0.9 mL, 3.6 mmol) at RT and the reaction mixture was stirred for 7 hours. The mixture was concentrated, diluted with water, basified with a 1M aqueous solution of NaOH and stirred at RT for 1 hour. The resultant precipitate was collected by filtration, rinsed with water and dried to afford the title compound 10 (111 mg, quant.) as a brown solid. ¹H NMR (400 MHz, DMSO-d6) δ (ppm): 8.54-8.51 (m, 1H), 8.51 (d, J=5.6 Hz, 1H), 8.31 (s, 1H), 8.23 (d, J=8.0 Hz, 1H), 7.84 (dd, J=8.0, 2.0 Hz, 1H), 7.81-7.75 (m, 2H), 7.61 (d, J=8.4 Hz, 1H), 7.48-7.41 (m, 3H), 7.06 (dd, J=8.4, 2.4 Hz, 1H), 6.66 (d, J=5.6 Hz, 1H), 3.52 (s, 2H), 2.79-2.70 (m, 2H), 2.58-2.44 (m, 1H), 2.04-1.94 (m, 2H), 1.71-1.62 (m, 2H), 1.30-1.20 (m, 2H). Three NH protons were missing. MS (m/z): 583.42 (M+H).

### Step 4. 2-(146-(7-(2-(4-Chlorophenylamino)benzo[d]oxazol-5-yloxy)thieno[3,2-b]pyridin-2-yl)pyridin-3-yl)methyl)piperidin-4-ylamino)-2-oxoethyl acetate (11)

To a stirred solution of 10 (58 mg, 0.0995 mmol) in DMF (3 mL) were added acetoxyacetic acid (23 mg, 0.199 mmol), EDC hydrochloride (38 mg, 0.199 mmol), HOBT monohydrate (23 mg, 0.149 mmol) and triethylamine (41 μL, 0.299 mmol) at RT and the reaction mixture was stirred for 1 hour. The reaction was then quenched by addition of water and the resultant precipitate was collected by filtration, rinsed with water, dried and purified by Biotage (MeOH/DCM: 0/100-20/80), to afford the title compound 11 (51 mg, 0.075 mmol, 75% yield) as a white solid. ¹H NMR (400 MHz, DMSO-d6) δ (ppm): 10.98 (s, 1H), 8.53 (d, J=1.6 Hz, 1H), 8.51 (d, J=5.6 Hz, 1H), 8.33 (s, 1H), 8.24 (d, J=8.0 Hz, 1H), 7.92 (d, J=7.6 Hz, 1H), 7.85 (dd, J=8.0, 2.0 Hz, 1H), 7.83-7.76 (m, 2H), 7.63 (d, J=8.8 Hz, 1H), 7.49-7.41 (m, 3H), 7.08 (dd, J=8.4, 2.4 Hz, 1H), 6.66 (d, J=5.2 Hz, 1H), 4.40 (s, 2H), 3.63-3.51 (m, 1H), 3.54 (s, 2H), 2.81-2.73 (m, 2H), 2.12-2.00 (m, 2H), 2.07 (s, 3H), 1.74-1.65 (m, 2H), 1.51-1.39 (m, 2H). MS (m/z): 683.44 (M+H).

### Step 5. N-(1-((6-(7-(2-(4-Chlorophenylamino)benzo[d]oxazol-5-yloxy)thieno[3,2-b]pyridin-2-yl)pyridin-3-yl)methyl)piperidin-4-yl)-2-hydroxyacetamide (12, example 1)

To a stirred solution of 11 (51 mg, 0.075 mmol) in MeOH/H₂O (6/1 mL) was added 3N NaOH (50 μL). The reaction mixture was stirred at RT for 3 days, concentrated and diluted with water. The resultant precipitate was collected by filtration, rinsed with water and dried to afford the title compound 12 (38 mg, 0.059 mmol, 80% yield) as a white solid. ¹H NMR (400 MHz, DMSO-d6) δ (ppm): 10.99 (s, 1H), 8.53 (d, J=1.6 Hz, 1H), 8.51 (d, J=5.2 Hz, 1H), 8.33 (s, 1H), 8.25 (d, J=8.0 Hz, 1H), 7.85 (dd, J=8.0, 2.0 Hz, 1H), 7.83-7.76 (m, 2H), 7.63 (d, J=8.4 Hz, 1H), 7.56 (d, J=8.4 Hz, 1H), 7.48-7.42 (m, 3H), 7.09 (dd, J=8.4, 2.4 Hz, 1H), 6.65 (d, J=5.6 Hz, 1H), 5.43 (t, J=6.0 Hz, 1H), 3.77 (d, J=6.0 Hz, 2H), 3.67-3.55 (m, 1H), 3.53 (s, 2H), 2.82-2.74 (m, 2H), 2.10-2.00 (m, 2H), 1.70-1.63 (m, 2H), 1.59-1.48 (m, 2H). MS (m/z): 641.40 (M+H).

Compound 13 (example 2) was prepared from 10 (scheme 2) by reacting it with ethyl isocyanate. Compound 14 (example 3) was prepared from compound 8 (scheme 2) using the procedures similar to the described above for the synthesis of compound 12 (example 1) and using in the reductive amination step N-Boc-N-methylpiperidin-4-amine instead of 4-N-Boc-aminopiperidine. Compound 15 (example 4) was prepared from compound 8 (scheme 2) using the procedures similar to the described above for the synthesis of compound 13 (example 2).

TABLE 1
Characterization of compounds 13-15 (examples 2-4).

| Cpd | Ex. | Structure | Characterization |
|---|---|---|---|
| 13 | 2 | 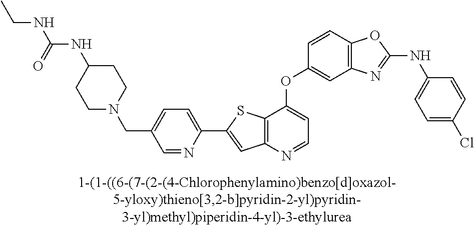 [SMILES: CCNC(=O)NC1CCN(Cc2ccc(-c3cc4nccc(Oc5ccc6oc(Nc7ccc(Cl)cc7)nc6c5)c4s3)nc2)CC1] | ¹H NMR (400 MHz, DMSO-d₆) δ (ppm): 10.98 (s, 1H), 8.53 (brs, 1H), 8.51 (d, J = 5.2 Hz, 1H), 8.33 (s, 1H), 8.24 (d, J = 8.0 Hz, 1H), 7.90-7.82 (m, 1H), 7.82-7.75 (m, 2H), 7.63 (d, J = 8.4 Hz, 1H), 7.50-7.42 (m, 3H), 7.08 (dd, J = 8.4, 2.4 Hz, 1H), 6.66 (d, J = 5.6 Hz, 1H), 5.73 (d, J = 8.0 Hz, 1H), 5.67 (t, J = 5.6 Hz, 1H), 3.53 (s, 2H), 3.4.-3.30 (m, 1H), 3.02-2.93 (m, 2H), 2.77-2.65 (m, 2H), 2.13- 2.02 (m, 2H), 1.77-1.68 (m, 2H), 1.38-1.25 (m, 2H), 0.96 (t, J = 7.2 Hz, 3H). MS (m/z): 654.57 (M + 1). |
| 14 | 3 | 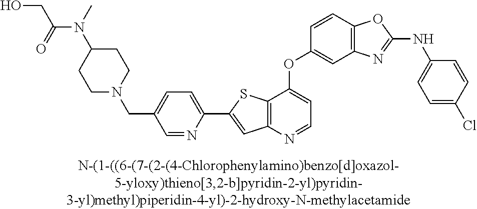 [SMILES: CN(C(=O)CO)C1CCN(Cc2ccc(-c3cc4nccc(Oc5ccc6oc(Nc7ccc(Cl)cc7)nc6c5)c4s3)nc2)CC1] | ¹H NMR (400 MHz, DMSO-d6) δ (ppm): 10.99 (s, 1H), 8.55 (d, J = 1.6 Hz, 1H), 8.51 (d, J = 5.6 Hz, 1H), 8.33 (s, 1H), 8.25 (d, J = 8.0 Hz, 1H), 7.86 (dd, J = 8.0, 2.0 Hz, 1H), 7.83-7.77 (m, 2H), 7.63 (d, J = 8.8 Hz, 1H), 7.49-7.42 (m, 3H), 7.08 (dd, J = 8.4, 2.4 Hz, 1H), 6.66 (d, J = 5.6 Hz, 1H), 4.45, 4.37 (t, J = 5.2 Hz, 1H, rotamer), 4.30-4.19, 3.48-3.30 (m, 1H, rotamer), 4.11, 4.03 (d, J = 5.2 Hz, 2H, rotamer), 3.57, 3.56 (s, 2H, rotamer), 2.92-2.85 (m, 2H), 2.74, 2.72 (s, 3H, rotamer), 2.12-2.03 (m, 2H), 1.84-1.65 (m, 2H), 1.60- 1.40 (m, 2H). MS (m/z): 655.44 (MH)+ |
| 15 | 4 | 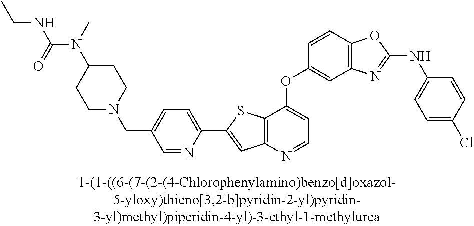 [SMILES: CCNC(=O)N(C)C1CCN(Cc2ccc(-c3cc4nccc(Oc5ccc6oc(Nc7ccc(Cl)cc7)nc6c5)c4s3)nc2)CC1] | ¹H NMR (400 MHz, DMSO-d₆) δ (ppm): 10.98 (s, 1H), 8.54 (d, J = 1.6 Hz, 1H), 8.51 (d, J = 5.6 Hz, 1H), 8.33 (s, 1H), 8.24 (d, J = 8.4 Hz, 1H), 7.86 (dd, J = 8.0, 2.0 Hz, 1H), 7.83-7.76 (m, 2H), 7.63 (d, J = 8.4 Hz, 1H), 7.49-7.42 (m, 3H), 7.08 (dd, J = 8.8, 2.4 Hz, 1H), 6.20 (t, J = 5.6 Hz, 1H), 3.97- 3.86 (m, 1H), 3.55 (s, 2H), 3.07-2.98 (m, 2H), 2.90-2.81 (m, 2H), 2.62 (s, 3H), 2.09-1.99 (m, 2H), 1.70-1.67 (m, 2H), 1.47-1.38 (m, 2H), 0.99 (t, J = 7.2 Hz, 3H). MS (m/z): 668.60 (M + 1). |

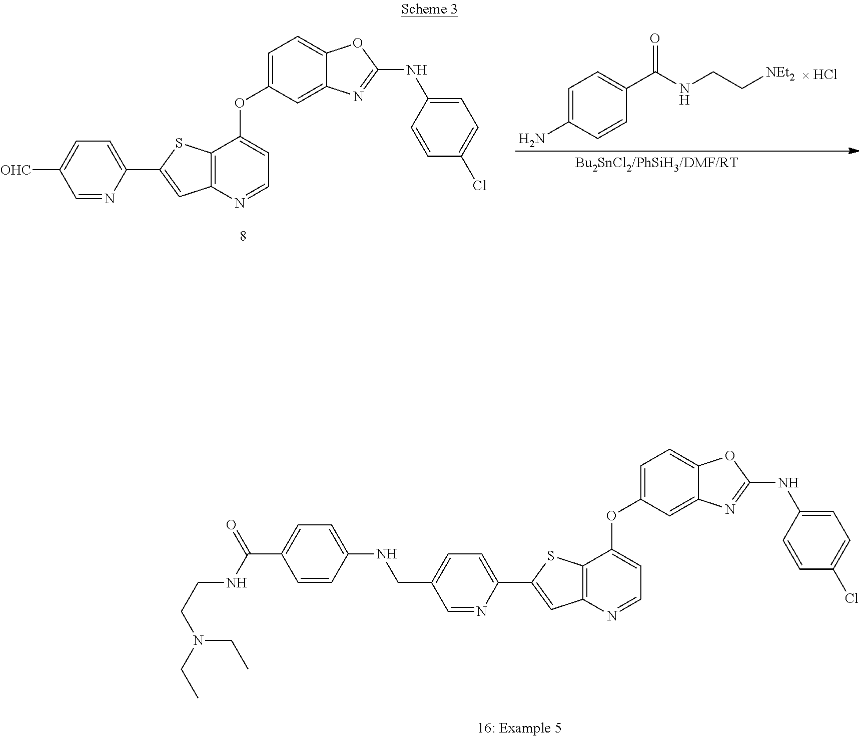

### Example 5

### 4-((6-(7-(2-(4-Chlorophenylamino)benzo[d]oxazol-5-yloxy)thieno[3,2-b]pyridin-2-yl)pyridin-3-yl)methyl)amino-N-(2-(diethylamino)ethyl)benzamide (16, example 5)

To a suspension of aldehyde 8 (0.178 g, 0.357 mmol), the amine salt 0.147 g, 0.541 mmol) and the dibutyltin dichloride (0.197 g, 0.648 mmol) in DMF was added phenylsilane (0.047 g, 0.434 mmol). The suspension became a solution and the reaction mixture was stirred for 2.5 hours. The reaction mixture was then poured into a mixture of brine/saturated NaHCO₃ solution and the resultant precipitate was collected by filtration and dried. The dry material was absorbed on silica gel and purified by flash column chromatography, eluent MeOH (16%) in DCM (methanol contained 2% ammonia), to afford title compound 16 (0.134 g, 52.3%). Characterization of 16 is provided in the table 2.

Compounds 17-20 (examples 6-8) were prepared starting from aldehyde 8 and using the procedure similar to the one described above for the synthesis of compound 16 (example 5).

TABLE 2
Characterization of compounds 16-20 (examples 5-8)

| Cpd | Ex. | Structure | Characterization |
|---|---|---|---|
| 16 | 5 | 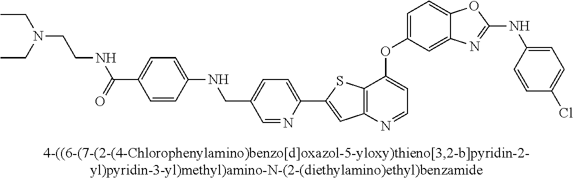 [SMILES: CCN(CC)CCNC(=O)c1ccc(NCc2ccc(-c3cc4nccc(Oc5ccc6oc(Nc7ccc(Cl)cc7)nc6c5)c4s3)nc2)cc1] | ¹H NMR (400 MHz, DMSO-d₆) δ (ppm): 10.98 (s, 1H); 8.63 (d, J = 1.8 Hz, 1H); 8.50 (d, J = 5.5, 1H); 8.31 (s, 1H); 8.24 (d, J = 8.2 Hz, 1H); 7.94 (t, J = 5.7 Hz, 1H); 7.89 (dd, J = 8.0, 2.0 Hz, 1H); 7.81-7.77 (m, 2H); 7.63 (d, J = 8.6 Hz, 1H); 7.59 (d, J = 8.8 Hz, 2H); 7.46-7.43 (m, 3H); 7.08 (dd, J = 8.6, 2.5 Hz, 1H); 6.86 (t, J = 6.3 Hz, 1H); 6.65-6.61 (m, 3H); 4.42 (d, J = 5.7 Hz, 2H); 3.25 (q, J = 14.1, 6.3 Hz, 4H); 0.96 (t, J = 7.0 Hz, 6H). MS (m/z): 718.5 (M + 1). |
| 17 | 6 | 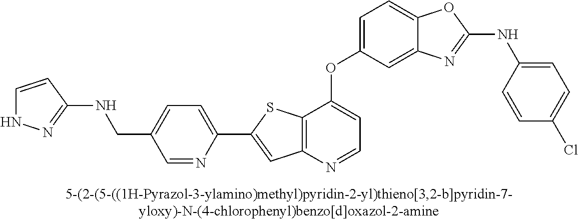 [SMILES: Clc1ccc(Nc2nc3cc(Oc4ccnc5cc(-c6ccc(CNc7cc[nH]n7)cn6)sc45)ccc3o2)cc1] | ¹H NMR (400 MHz, DMSO-d₆) δ (ppm): 11.55 (br. s, 1H); 10.98 (s, 1H); 8.59 (d, J = 1.6 Hz, 1H); 8.50 (d, J = 5.5, 1H); 8.29 (s, 1H); 8.21 (d, J = 8.0 Hz, 1H); 7.89 (dd, J = 8.2, 2.2 Hz, 1H); 7.81-7.77 (m, 2H); 7.63 (d, J = 8.6 Hz, 1H); 7.47-7.43 (m, 3H); 7.34 (br. d, J = 1.6 Hz, 1H); 7.08 (dd, J = 8.6, 2.5 Hz 1H); 6.64 (d, J = 5.3 Hz 1H); 5.65 (br. s, 1H); 5.49 (br. s, 1H); 4.29 (d, J = 6.3 Hz, 2H). MS (m/z): 566.3 (M + 1). |
| 18 | 7 | 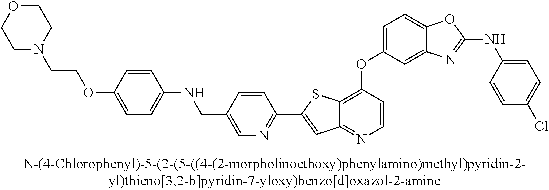 [SMILES: Clc1ccc(Nc2nc3cc(Oc4ccnc5cc(-c6ccc(CNc7ccc(OCCN8CCOCC8)cc7)cn6)sc45)ccc3o2)cc1] | ¹H NMR (400 MHz, DMSO-d₆) δ (ppm): 10.95 (s, 1H), 8.59 (m, 1H), 8.48 (d, J = 5.49 Hz, 1H), 8.27 (s, 1H), 8.19 (d, J = 8.22 Hz, 1H), 7.87 (m, 1H), 7.78 (m, 2H), 7.61 (d, J = 8.61 Hz, 1H), 7.40 (m, 3H), 7.02 (m, 1H), 6.68 (m, 2H), 6.63 (m, 1H), 6.52 (m, 2H), 5.92 (t, J = 6.26 Hz, 2H), 4.27 (d, J = 6.06 Hz, 2H), 3.89 (t, J = 5.87 Hz, 2H), 3.53 (m, 4H), 2.58 (t, J = 5.87 Hz, 2H), 2.39 (m, 4H). MS (m/z): 705.53/707.52 (M + 1). |
| 19 | 8 | 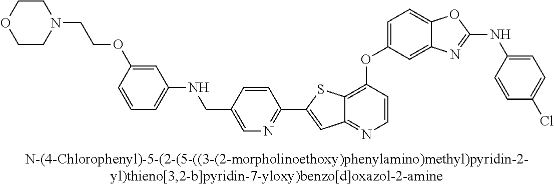 [SMILES: Clc1ccc(Nc2nc3cc(Oc4ccnc5cc(-c6ccc(CNc7cccc(OCCN8CCOCC8)c7)cn6)sc45)ccc3o2)cc1] | ¹H NMR (400 MHz, DMSO-d6) δ (ppm): 10.96 (s, 1H), 8.59 (m, 1H), 8.47 (d, J = 5.48 Hz, 1H), 8.28 (s, 1H), 8.21 (d, J = 8.22 Hz, 1H), 7.86 (m, 1H), 7.61 (d, J = 8.61 Hz, 1H), 7.44 (s, 1H), 7.42 (d, J = 7.04 Hz, 2H), 7.05 (m, 1H), 6.92 (m, 1H), 6.62 (d, J = 5.48 Hz, 1H), 6.33 (t, J = 5.86 Hz, 1H), 6.21 (m, 1H), 6.12 (m, 2H), 4.33 (m, 2H), 3.94 (t, J = 5.87 Hz, 2H), 3.53 (t, J = 4.50 Hz, 4H), 2.59 (t, J = 5.67 Hz, 2H), 2.48 (m, 4H). MS (m/z): 705.57/707.59 (MH)+ |

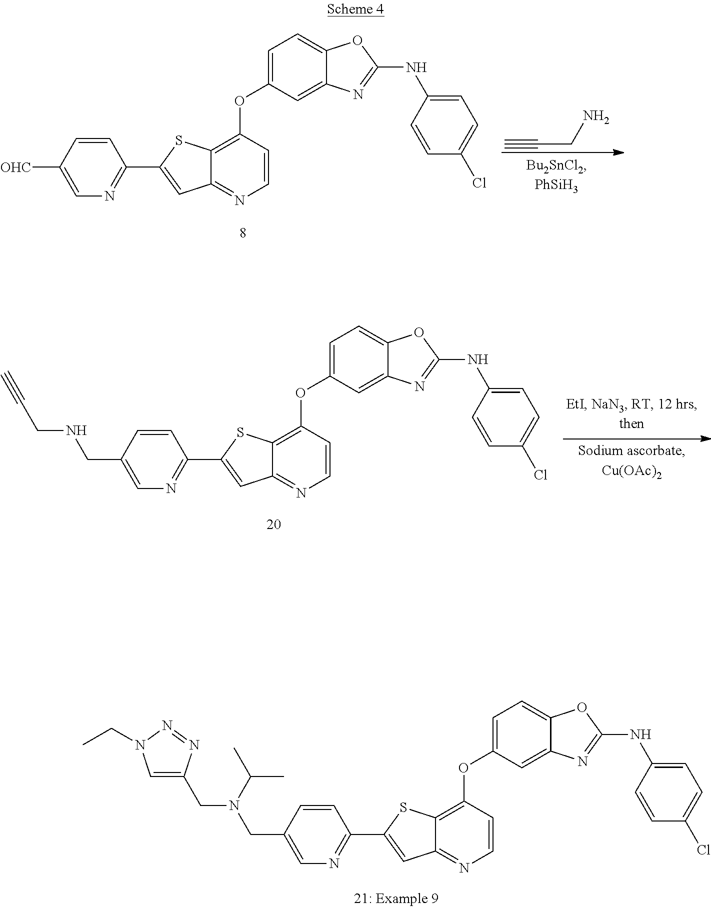

### Example 9

### N-(4-Chlorophenyl)-5-(2-(5-((((1-ethyl-1H-1,2,3-triazol-4-yl)methyl)(isopropyl)amino)methyl)pyridin-2-yl)thieno[3,2-b]pyridin-7-yloxy)benzo[d]oxazol-2-amine (21, example 9)

### Step 1: N-(4-Chlorophenyl)-5-(2-(5-((prop-2-ynylamino)methyl)pyridin-2-yl)thieno[3,2-b]pyridin-7-yloxy)benzo[d]oxazol-2-amine (20)

To a solution of 8 (215 mg, 0.431 mmol) in DMF (3 mL) was added propargyl amine (35.6 mg, 1.5 eq, 0.646 mmol), dibutyltin dichloride (157 mg, 1.2 eq, 0.517 mmol) and phenylsilane (84 mg, 1.8 eq, 0.776 mmol). The reaction mixture was stirred at RT overnight, diluted with EtOAc and washed with water. The organic phase was, dried over anhydrous MgSO₄, filtered and concentrated. Purification by column chromatography (EtOAc to 20% MeOH in EtOAc) afforded 20 (170 mg, 73%) as a white solid. MS (m/z)=539.12/541.12 (M+H).

### Step 2: N-(4-Chlorophenyl)-5-(2-(5-((((1-ethyl-1H-1,2,3-triazol-4-yl)methyl)(isopropyl)amino)methyl)pyridin-2-yl)thieno[3,2-b]pyridin-7-yloxy)benzo[d]oxazol-2-amine (21, example 9)

To a solution of iodoethane (99 mg, 2 eq, 3.27 mmol) in DMSO (3 mL) was added sodium azide (41.1 mg, 2 eq, 0.632 mmol) and the reaction mixture was stirred for 24 hrs at RT. Compound 20 (170 mg, 0.316 mmol), Cu(OAc)₂ (17.22 mg, 0.3 eq, 0.095 mmol) and sodium ascorbate (37.6 mg, 0.6 eq, 0.19 mmol) were added and the reaction mixture was allowed to stir for an additional hour. The reaction mixture was poured into water/NH₄OH (pH ˜10) whereupon a solid precipitated. The mixture was stirred with DCM/MeOH to dissolve the solid; the organic phase was collected, dried over anhydrous MgSO₄, filtered and concentrated. Purification by column chromatography (5% MeOH in EtOAc) afforded 21 (31 mg, 15% yield) as a white solid. ¹H NMR (400 MHz, DMSO-d6) δ (ppm): 10.96 (s, 1H), 8.54 (m, 1H), 8.48 (m, 1H), 8.28 (s, 1H), 8.16 (d, J=8.41 Hz, 1H), 7.98 (s, 1H), 7.83 (m, 1H), 7.77 (d, J=8.99 Hz, 1H), 7.61 (8.61 Hz, 1H), 7.43 (d, J=8.61 Hz, 1H), 7.05 (m, 1H), 6.64 (d, 5.48 Hz, 1H) 4.3 (q, J=7.43 Hz, 2H), 3.63 (s, 4H), 2.89 (m, 1H), 1.35 (t, J=7.34 Hz, 3H), 1.03 (d, J=6.46 Hz, 6H). MS (m/z) 651.61 (MH)⁺

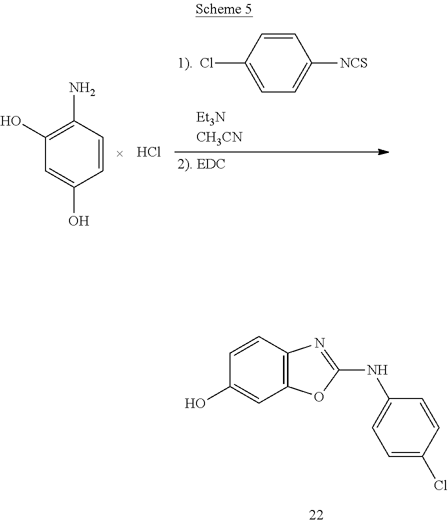

### 2-(4-Chlorophenylamino)benzo[d]oxazol-6-ol (22)

To a suspension of 2-aminoresorcinol×HCl (484 mg, 3.0 mmol) and Et₃N (303 mg, 3.0 mmol) in MeCN (30 mL) was added chlorophenyl thioisocyanate (610 mg, 3.6 mmol). The reaction mixture was stirred for 1 week at room temperature then EDC×HCl (575 mg, 3.0 mmol) was added and stirring was continued for 1 day. The reaction mixture was concentrated and the residue purified by flash column chromatography (Hexane/AcOEt=90/10-60/40) to give a material that upon trituration with CH₂Cl₂ afforded title compound 22 (283 mg, 36% yield) as a pale pink solid. ¹H-NMR (CD₃OD) δ: 7.66-7.59 (m, 2H), 7.34-7.28 (m, 2H), 7.19 (d, J=8.4 Hz, 1H), 6.82 (d, J=2.4 Hz, 1H), 6.69 (dd, J=8.4, 2.4 Hz, 1H).

Compounds 23-32 were prepared either similarly to compound 4 (scheme 1) or 22 (scheme 5) starting either from 3-amino-4-hydroxyphenyl benzoate or 2-aminoresorcinol and using appropriate commercially available isothiocyanates.

TABLE 3
Characterization of compounds 23-32

| Cpd | Structure | Characterization |
|---|---|---|
| 23 | 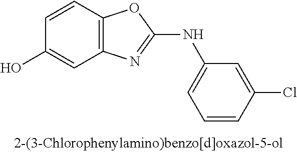 [SMILES: Oc1ccc2oc(Nc3cccc(Cl)c3)nc2c1] | ¹H-NMR (CD₃OD) δ: 7.78 (d, J = 1.5 Hz, 1H), 7.44 (d, J = 8.1 Hz, 1H), 7.25 (m, 1H), 7.13 (d, J = 8.1 Hz, 1H), 7.00 (d, J = 7.5 Hz, 1H), 6.86 (s, 1H), 6.57 (d, J = 8.7 Hz, 1H). |
| 24 | 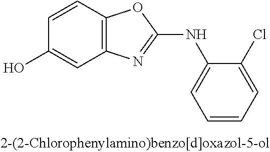 [SMILES: Oc1ccc2oc(Nc3ccccc3Cl)nc2c1] | ¹H-NMR (CD₃OD) δ: 8.07 (d, J = 8.1 Hz, 1H), 7.47 (dd, J = 7.8, 1.5 Hz, 1H), 7.39-7.33 (m, 1H), 7.19-7.11 (m, 2H), 6.81 (d, J = 2.4 Hz, 1H), 6.57 (dd, J = 8.4, 2.4 Hz, 1H). |
| 25 | 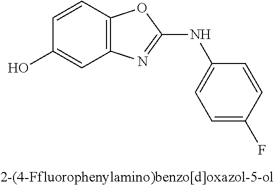 [SMILES: Oc1ccc2oc(Nc3ccc(F)cc3)nc2c1] | ¹H-NMR (CD₃OD) δ: 7.63-7.58 (m, 2H), 7.15-7.05 (m, 3H), 6.81 (d, J = 2.4 Hz, 1H), 6.55 (dd, J = 8.4, 2.4 Hz, 1H). |
| 26 | 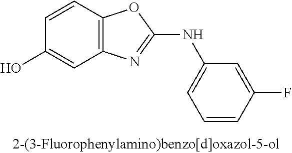 [SMILES: Oc1ccc2oc(Nc3cccc(F)c3)nc2c1] | ¹H-NMR (CD₃OD) δ: 7.62-7.56 (m, 1H), 7.33-7.28 (m, 2H), 7.16 (d, J = 8.4 Hz, 1H), 6.86 (d, J = 2.4 Hz, 1H), 6.79-6.72 (m, 1H), 6.58 (dd, J = 8.4, 2.4 Hz, 1H). |
| 27 | 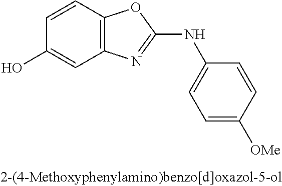 [SMILES: COc1ccc(Nc2nc3cc(O)ccc3o2)cc1] | ¹H-NMR (CD₃OD) δ: 7.48-7.45 (m, 2H), 7.10 (d, J = 8.4 Hz, 1H) , 6.93-6.90 (m, 2H), 6.77 (d, J = 2.4 Hz, 1H), 6.52 (dd, J = 8.4, 2.4 Hz, 1H), 3.78 (s, 3H). |
| 28 | 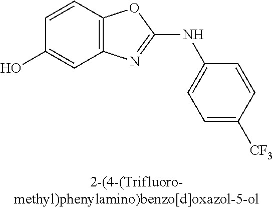 [SMILES: Oc1ccc2oc(Nc3ccc(C(F)(F)F)cc3)nc2c1] | ¹H-NMR (CD₃OD) δ: 7.84-7.80 (m, 2H), 7.63-7.58 (m, 2H), 7.17 (d, J = 8.4 Hz, 1H), 6.88 (d, J = 2.4 Hz, 1H), 6.60 (dd, J = 8.4, 2.4 Hz, 1H). |
| 29 | 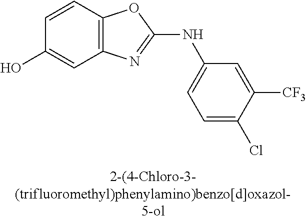 [SMILES: Oc1ccc2oc(Nc3ccc(Cl)c(C(F)(F)F)c3)nc2c1] | ¹H-NMR (CD₃OD) δ: 11.0 (s, 1H), 9.30 (s, 1H), 8.25 (s, 1H), 8.06 (d, J = 8.7 Hz, 1H), 7.72 (d, J = 8.7 Hz, 1H), 7.30 (d, J = 8.7 Hz, 1H), 6.85 (d, J = 2.4 Hz, 1H), 6.58 (dd, J = 8.7, 2.4 Hz, 1H). |
| 30 | 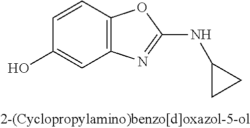 [SMILES: Oc1ccc2oc(NC3CC3)nc2c1] | ¹H-NMR (DMSO-d₆) δ: 9.03 (s, 1H), 8.00 (d, J = 2.4 Hz, 1H), 7.10 (d, J = 8.4 Hz, 1H), 6.65 (d, J = 2.4 Hz, 1H), 6.37 (dd, J = 8.4, 2.4 Hz, 1H), 2.69 (oct, J = 3.3 Hz, 1H), 0.74- 0.68 (m, 2H), 0.56-0.51 (m, 2H). |
| 31 | 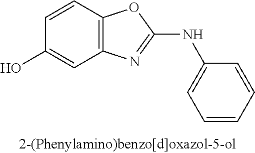 [SMILES: Oc1ccc2oc(Nc3ccccc3)nc2c1] | ¹H-NMR (DMSO-d₆) δ: 10.47 (s, 1H), 9.20 (s, 1H), 7.73 (d, J = 8.1 Hz, 2H), 7.36 (dd, J = 8.1, 7.5 Hz, 2H), 7.24 (d, J = 8.4 Hz, 1H), 7.02 (t, J = 7.5 Hz, 1H), 6.81 (d, J = 2.1 Hz, 1H), 6.52 (dd, J = 8.4, 2.1 Hz, 1H). |
| 32 | 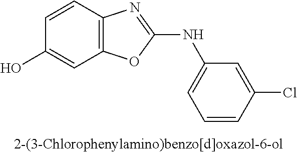 [SMILES: Oc1ccc2nc(Nc3cccc(Cl)c3)oc2c1] | ¹H-NMR (DMSO-d₆) δ: 7.83-7.81 (m, 1H), 7.48-7.44 (m, 1H), 7.30 (m, 1H), 7.23 (d, J = 8.4 Hz, 1H), 7.02-6.99 (m, 1H), 6.83 (d, J = 2.4 Hz, 1H), 6.70 (dd, J = 8.4, 2.4 Hz, 1H). |

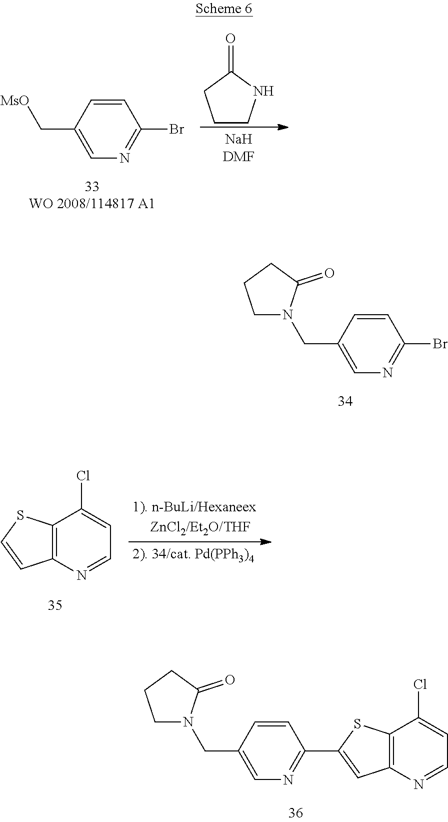

### 1-((6-(7-Chlorothieno[3,2-b]pyridin-2-yl)pyridin-3-yl)methyl)pyrrolidin-2-one (36)

### Step 1: 1((6-Bromopyridin-3-yl)methyl)pyrrolidin-2-one (34)

To a solution of 2-pyrrolidone (8.51 g, 0.10 mol) in DMF (50 mL) was added portionwise NaH (60% in mineral oil, 4.00 g, 0.10 mol) at 5° C., and the reaction mixture was stirred at 5° C. for 20 min. To the reaction mixture was added dropwise a solution of mesylate 33 (19.80 g, 0.074 mol, WO 2008/114817 A1) in DMF (27 mL) over 15 min at 5-25° C. The reaction mixture was stirred at room temperature for 1 hour, quenched by addition of water (30 mL) and concentrated. The residue was extracted with EtOAc and the extract was washed with water, brine, dried over MgSO₄ and concentrated in vacuo to afford title compound 34 (15.3 g, 78% yield) as a pale yellow oil. ¹H-NMR (300 MHz, CDCl₃) δ (ppm): 8.27 (d, J=1.5 Hz, 1H), 7.50 (dd, J=8.1, 2.1 Hz, 1H), 7.46 (dd, J=8.1, 1.5 Hz, 1H), 4.42 (s, 2H), 3.28 (t, J=7.2 Hz, 2H), 2.44 (t, J=8.1 Hz, 2H), 2.09-1.98 (m, 2H).

### Step 2: 1-((6-(7-Chlorothieno[3,2-b]pyridin-2-yl)pyridin-3-yl)methyl)pyrrolidin-2-one (36)

To a solution of 7-chlorothieno[3,2-b]pyridine (35) (1.87 g, 0.011 mol) in THF (55 mL) was added n-BuLi (2.6M in hexane, 4.61 mL, 0.020 mol) over 5 min at −78-−70° C., and the mixture was stirred for 20 min at −70° C. To the reaction mixture was added ZnCl₂ (1.0M in ether, 12 mL, 0.012 mol) over 5 min at −78-−70° C. The combined reaction mixture was stirred for 20 min at −70° C. then allowed to warm to room temperature. To the reaction mixture was added compound 34 (2.55 g, 0.010 mol) and Pd(PPh₃)₄ (0.58 g, 0.50 mmol), and the resultant mixture was heated to reflux for 2 hours. After cooling to room temperature, the mixture was concentrated in vacuo. The residue was purified by flash chromatography on silica gel (NH silica, eluent EtOAc/MeOH), to afford a material that was triturated with EtOAc, to afford title compound 36 as a colorless solid (2.94 g, 85% yield). ¹H-NMR (300 MHz, DMSO-d₆) δ (ppm): 8.66 (d, J=5.1 Hz, 1H), 8.54 (d, J=2.4 Hz, 1H), 8.43 (s, 1H), 8.29 (d, J=8.4 Hz, 1H), 7.82 (dd, J=8.4, 2.1 Hz, 1H), 7.59 (d, J=5.1 Hz, 1H), 4.47 (s, 2H), 3.31 (t, J=6.9 Hz, 2H), 2.32 (t, J=7.8 Hz, 2H), 2.01-1.90 (m, 2H).

### Scheme 7

### (7-Chlorothieno[3,2-b]pyridin-2-yl)(piperidin-1-yl)methanone (38) and (S)-(7-chlorothieno[3,2-b]pyridin-2-yl)(3-(dimethylamino)pyrrolidin-1-yl)methanone (39)

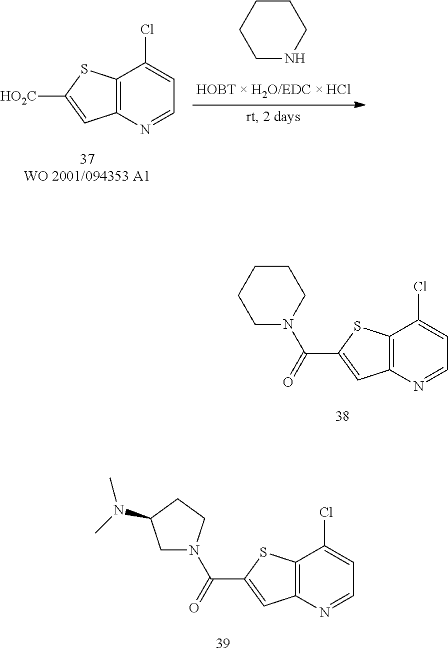 [SMILES: O=C(O)c1cc2nccc(Cl)c2s1 ; C1CCNCC1 ; O=C(c1cc2nccc(Cl)c2s1)N1CCCCC1 ; CN(C)[C@H]1CCN(C(=O)c2cc3nccc(Cl)c3s2)C1]

### (7-Chlorothieno[3,2-b]pyridin-2-yl)(piperidin-1-yl)methanone (38)

To the solution of 7-chlorothieno[3,2-b]pyridine-2-carboxylic acid (37) (1.07 g, 5.0 mmol, WO 2001/094353 A1), EDC×HCl (1.05 g, 5.5 mmol) and HOBt (676 mg, 5.0 mmol) in DMSO/MeCN (1:1, 40 mL) was added piperidine (5.0 mmol) at room temperature and the reaction mixture was stirred for 2 days. The reaction was quenched with water (150 mL) and the resultant solution was extracted with AcOEt (150 mL×3). The extract was dried over anhydrous MgSO₄, filtered and concentrated under reduced pressure. The residue was purified by flash column purification (eluent Hexane/AcOEt 90:10-10/90) to afford the title compound 38 (958 mg, 68% yield) as a pale yellow oil (MAP-13138, 68% yield). ¹H-NMR (CDCl₃) δ: 8.62 (d, J=5.1 Hz, 1H), 7.65 (s, 1H), 7.34 (d, J=5.1 Hz, 1H), 3.75-3.63 (m, 4H), 1.72-1.63 (m, 6H).

### (S)-(7-Chlorothieno[3,2-b]pyridin-2-yl)(3-(dimethylamino)pyrrolidin-1-yl)methanone (39)

Tile compound 39 was obtained from the acid 37 and (S)—N,N-dimethylpyrrolidin-3-amine by following the procedure described above for the synthesis of amide 38. ¹H-NMR (CDCl₃) δ: 8.63 (d, J=5.1 Hz 1H), 7.86 (s, 1H), 7.36 (d, J=5.1 Hz, 1H), 4.10-3.45 (m, 4H), 2.88-2.76 (m, 1H), 2.32 (s, 3H), 2.30 (s, 3H), 2.28-2.18 (m, 1H), 2.05-1.80 (m, 1H).

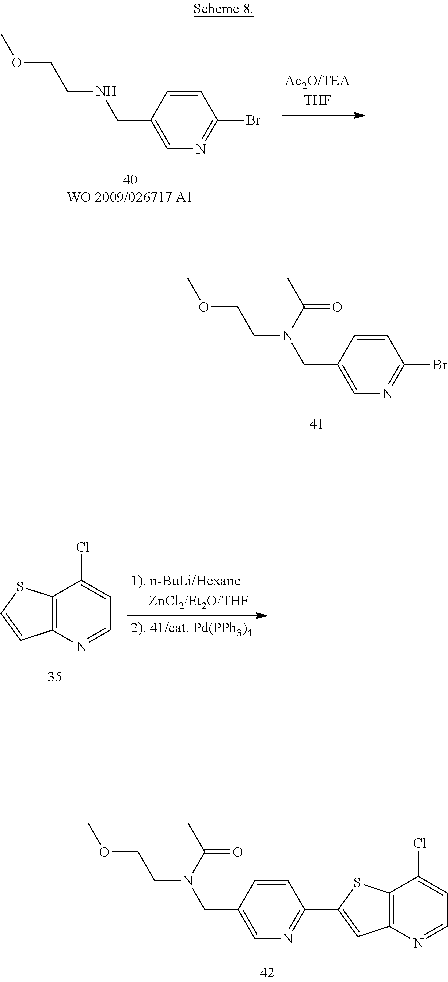 [SMILES: COCCNCc1ccc(Br)nc1 ; COCCN(Cc1ccc(Br)nc1)C(C)=O ; Clc1ccnc2ccsc12 ; COCCN(Cc1ccc(-c2cc3nccc(Cl)c3s2)nc1)C(C)=O]

### N-((6-(7-Chlorothieno[3,2-b]pyridin-2-yl)methyl)-N-(2-methoxethyl)acetamide (42)

### Step 1: N-((6-bromopyridin-3-yl)methyl)-N-(2-methoxyethyl)acetamide (41)

To a solution of N-((6-bromopyridin-3-yl)methyl)-2-methoxyethanamine (40) (5.94 g, 0.024 mol, WO 2009/026717 A1), TEA (3.68 g, 0.036 mol) in THF (48 mL) was added dropwise Ac₂O (3.00 g, 0.029 mol) at room temperature. The reaction mixture was stirred at room temperature for 1 hour, quenched with aqueous NaHCO₃ solution, and extracted with EtOAc. The organic extract was collected, washed with brine, dried over MgSO₄ and concentrated in vacuo to afford title compound 41 (6.74 g, 97% yield) as a brown oil which was used in the next step without further purification. ¹H-NMR (300 MHz, CDCl₃) δ (ppm): 8.28 (d, J=2.7 Hz, 0.7H), 8.26 (d, J=2.7 Hz, 0.3H), 7.54 (dd, J=8.4, 2.7 Hz, 0.7H), 7.51 (d, J=8.4 Hz, 0.3H), 7.44 (d, J=8.4 Hz, 0.7H), 7.40 (dd, J=8.4, 2.7 Hz, 0.3H), 4.66 (s, 0.6H), 4.61 (s, 1.4H), 3.55 (s, 1.2H), 3.45 (s, 2.8H), 3.30 (s, 3H), 2.22 (s, 2.1H), 2.14 (s, 0.9H).

### Step 2: N-((6-(7-Chlorothieno[3,2-b]pyridin-2-yl)pyridin-3-yl)methyl)-N-(2-methoxyethyl)acetamide (42)

To a solution of 7-chlorothieno[3,2-b]pyridine (35) (3.10 g, 0.018 mol) in THF (37 mL) was added n-BuLi (2.6M in hexane, 7.70 mL, 0.020 mol) over 10 min at −70-−60° C., and the reaction mixture was stirred for 40 min at −70° C. To the reaction mixture was added ZnCl₂ (1.0M in ether, 20 mL, 0.020 mol) over 10 min at −70-−60° C. The combined reaction mixture was stirred for 20 min at −70° C. then allowed to warm to room temperature. To the reaction mixture was added a solution of bromide 41 (5.00 g, 0.017 mol) in THF (17 mL) and Pd(PPh₃)₄ (0.40 g, 0.35 mmol), and the reaction mixture was heated to reflux for 4 hours. After cooling to room temperature, a saturated aqueous NH₄Cl solution was added, and the mixture was extracted with EtOAc. The extract was washed with brine, dried over MgSO₄, filtered and concentrated in vacuo. The residual material was triturated with EtOAc, to afford title compound 42 (3.50 g, 54% yield) as a pale yellow solid. ¹H-NMR (300 MHz, DMSO-d₆) δ (ppm): 8.67 (d, J=5.1 Hz, 0.3H), 8.66 (d, J=5.1 Hz, 0.7H), 8.55-8.52 (m, 1H), 8.45 (s, 0.3H), 8.42 (s, 0.7H), 8.32 (d, J=8.1 Hz, 0.3H), 8.26 (d, J=8.1 Hz, 0.7H), 7.82-7.75 (m, 1H), 7.60 (d, J=5.1 Hz, 0.3H), 7.59 (d, J=5.1 Hz, 0.7H), 4.73 (s, 0.6H), 4.60 (s, 1.4H), 3.54-3.41 (m, 4H), 3.24 (s, 2.1H), 3.21 (s, 0.9H), 2.13 (s, 2.1H), 2.05 (s, 0.9H).

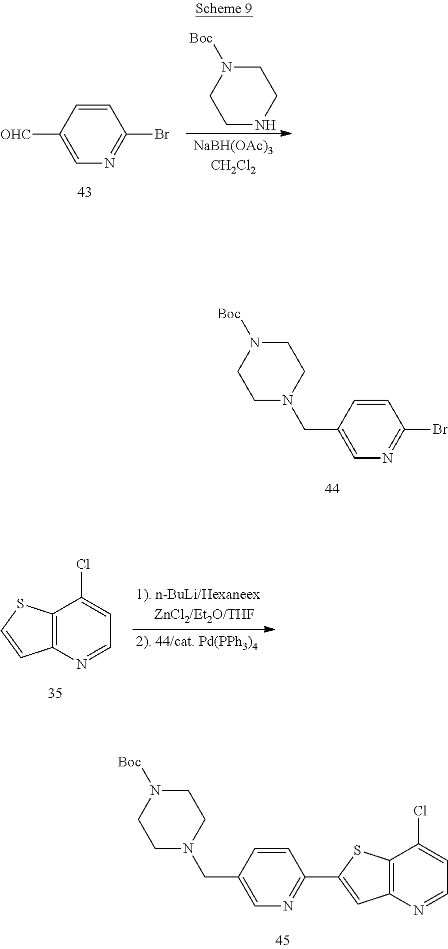 [SMILES: O=Cc1ccc(Br)nc1 ; CC(C)(C)OC(=O)N1CCNCC1 ; CC(C)(C)OC(=O)N1CCN(Cc2ccc(Br)nc2)CC1 ; Clc1ccnc2ccsc12 ; CC(C)(C)OC(=O)N1CCN(Cc2ccc(-c3cc4nccc(Cl)c4s3)nc2)CC1]

### tert-Butyl 4-((6-(7-chlorothieno[3,2-b]pyridin-2-yl)pyridin-3-yl)methyl)piperazine-1-carboxylate (45)

### Step 1: tert-Butyl 4((6-bromopyridin-3-yl)methyl)piperazine-1-carboxylate (44)

To a solution of 6-bromonicotinaldehyde (43) (16.0 g, 0.086 mol), tert-butyl piperazine-1-carboxylate (19.2 g, 0.10 mol), AcOH (8.0 mL) in CH₂Cl₂ (320 mL) was added portion wise NaBH(OAc)₃ (23.0 g, 0.11 mol) at 10-13° C. The reaction mixture was stirred at room temperature for 16 hours, treated with saturated aqueous NaHCO₃ solution and extracted with CH₂Cl₂. The extract was washed with a saturated NaHCO₃ solution, brine, dried over MgSO₄, filtered and concentrated in vacuo. The residual solid was triturated with t-BuOMe (40 mL) to afford title compound 44 as a colorless solid (16.1 g, 52% yield). The filtrate after the trituration was concentrated, the residue was triturated with a mixture of t-BuOMe-hexane (1:1, 20 mL), to afford a second crop of title compound 44 (4.10 g, 14% yield). ¹H-NMR (300 MHz, CDCl₃) δ (ppm): 8.30 (d, J=2.4 Hz, 1H), 7.56 (dd, J=8.1, 2.4 Hz, 1H), 7.45 (d, J=8.1 Hz, 1H), 3.47 (s, 2H), 3.42 (t, J=5.1 Hz, 4H), 2.38 (t, J=5.1 Hz, 4H), 1.46 (s, 9H).

### Step 2: tert-Butyl 4-((6-(7-chlorothieno[3,2-b]pyridin-2-yl)pyridin-3-yl)methyl)piperazine-1-carboxylate (45)

To a solution of 7-chlorothieno[3,2-b]pyridine (35) (10.68 g, 0.063 mol) in THF (330 mL) was added n-BuLi (2.6M in hexane, 25.0 mL, 0.065 mol) over 10 min maintaining the temperature between −40 and −27° C. The reaction mixture was stirred for 40 min at −40° C. and treated with ZnCl₂ (1.9M in 2-methyltetrahydrofuran (34.5 mL, 0.066 mol) over 10 min maintaining the temperature between −40 and −27° C. The combined reaction mixture was stirred for 10 min at −40° C. then allowed to warm to room temperature. To the reaction mixture was added compound 44 (21.4 g, 0.060 mol) and Pd(PPh₃)₄ (0.69 g, 0.60 mmol), and the resultant mixture was heated to reflux for 40 min. After cooling to room temperature, saturated aqueous NH₄Cl solution (100 mL) was added, and the mixture was extracted with 2-methyltetrahydrofuran (100 mL). The organic extract was washed with brine, dried over MgSO₄, filtered and concentrated in vacuo. The residual solid was triturated with EtOAc-MeOH (9:1, 200 mL), collected by filtration and washed with EtOAc to afford title compound 45 (18.8 g, 70% yield) as a beige solid. The filtrate was concentrated, and the residue was triturated with a mixture EtOAc-MeOH (9:1, 20 mL), to afford a second crop of compound 45 as a beige solid (5.40 g, 21% yield). ¹H-NMR (300 MHz, DMSO-d₆) δ (ppm): 8.66 (dd, J=5.1, 0.6 Hz, 1H), 8.58 (s, 1H), 8.42 (s, 1H), 8.28 (d, J=8.1 Hz, 1H), 7.89 (d, J=8.1 Hz, 1H), 7.59 (dd, J=5.1, 0.6 Hz, 1H), 3.59 (s, 2H), 3.37-3.32 (m, 4H), 2.40-2.32 (m, 4H), 1.39 (s, 9H).

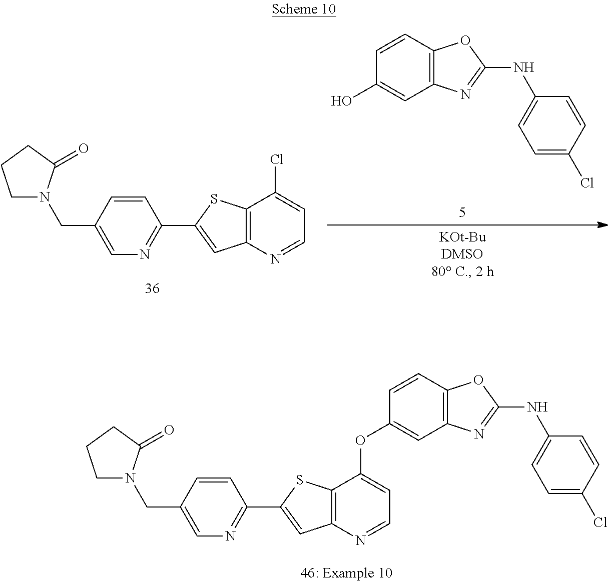 [SMILES: O=C1CCCN1Cc1ccc(-c2cc3nccc(Cl)c3s2)nc1 ; Oc1ccc2oc(Nc3ccc(Cl)cc3)nc2c1 ; O=C1CCCN1Cc1ccc(-c2cc3nccc(Oc4ccc5oc(Nc6ccc(Cl)cc6)nc5c4)c3s2)nc1]

### Example 10

### 1-((6-(7-(2-(4-Chlorophenylamino)benzo[d]oxazol-5-yloxy)thieno[3,2-b]pyridin-2-yl)pyridin-3-yl)methyl)pyrrolidin-2-one (46)

To hydroxylbenzoxazole 5 (156 mg, 0.60 mmol), chloride 36 (103 mg, 0.30 mmol) and KOtBu (79 mg, 0.70 mmol) was added anhydrous DMSO (3.0 mL) at room temperature under the argon atmosphere. The reaction was stirred for 30 min at room temperature then for 2 h at 80° C. The reaction was quenched with water at room temperature to form a precipitate that was collected by filtration and dried. The material was purified by column chromatography (NH silica, CH₂Cl₂/MeOH=100/0-90/5), to afford compound 46 (20 mg, 12% yield) as a colorless solid. ¹H-NMR (CDCl₃) δ (ppm): 8.54 (d, J=1.8 Hz, 1H), 8.48 (d, J=5.7 Hz, 1H), 8.00 (s, 1H), 7.86 (d, J=7.8 Hz, 1H), 7.72 (dd, J=7.8, 2.1 Hz, 1H), 7.62-7.58 (m, 2H), 7.40-7.33 (m, 4H), 7.18 (brs, 1H), 7.00 (dd, J=8.7, 2.4 Hz, 1H), 6.58 (d, J=5.7 Hz, 1H), 4.52 (s, 2H), 3.33 (dd, J=7.8, 7.2 Hz, 2H), 2.47 (dd, J=8.4, 7.8 Hz, 2H), 2.10-2.00 (m, 2H). MS (m/z): 568.3 (M+H)⁺.

Compounds 47-57 (examples 11-17) were obtained by reacting chloride 36 with the hydroxybenzoxazoles 22-32 (table 3), respectively.

Compounds 58 and 59 (examples 22 and 23) were obtained by reacting chloride 38 with the hydroxybenzoxazoles 30 and 31 (table 3), respectively.

Compounds 60 and 61 (examples 24 and 25) were obtained by reacting chloride 39 with the hydroxybenzoxazoles 30 and 31, (table 3) respectively.

Compounds 62-64 (examples 26-28) were obtained by reacting chloride 42 with the hydroxybenzoxazoles 5 (scheme 1), 30 and 31 (table 3), respectively.

Compound 65 (examples 29) was obtained by reacting chloride 45 with the hydroxybenzoxazole 5 (scheme 1).

TABLE 4
Characterization of compounds 47-65 (examples 11-29)

| Cpd | Ex. | Structure | Characterization |
|---|---|---|---|
| 47 | 11 | 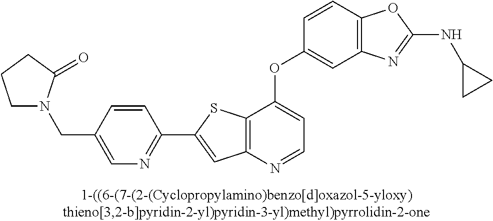 [SMILES: O=C1CCCN1Cc1ccc(-c2cc3nccc(Oc4ccc5oc(NC6CC6)nc5c4)c3s2)nc1] | ¹H-NMR (CDCl₃) δ (ppm): 8.54 (d, J = 2.0 Hz, 1H), 8.46 (d, J = 5.5 Hz, 1H), 7.86 (d, J = 8.4 Hz, 1H), 7.71 (d, J = 8.4, 2.0 Hz, 1H), 7.30 (d, J = 8.4 Hz, 1H), 7.23 (d, J = 2.7 Hz, 1H), 6.89 (dd, J = 8.4, 2.7 Hz, 1H), 6.55 (d, J = 5.5 Hz, 1H), 5.45 (brs, 1H), 4.51 (s, 2H), 3.33 (t, J = 6.9 Hz, 2H), 2.87-2.89 (m, 1H), 2.47 (t, J = 8.0 Hz, 2H), 2.10-2.02 (m, 2H), 0.95-0.89 (m, 2H), 0.77-0.71 (m, 2H). MS (m/z): 498.2 (M + H)⁺. |
| 48 | 12 | 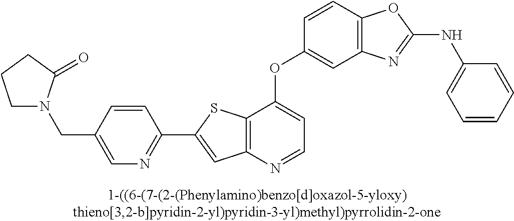 [SMILES: O=C1CCCN1Cc1ccc(-c2cc3nccc(Oc4ccc5oc(Nc6ccccc6)nc5c4)c3s2)nc1] | ¹H-NMR (DMSO-d₆) δ (ppm): 10.8 (s, 1H), 8.53-8.51 (m, 2H), 8.34 (s, 1H), 8.26 (d, J = 8.1 Hz, 1H), 7.81-7.75 (m, 3H), 7.62 (d, J = 9.0 Hz, 1H), 7.44 (d, J = 2.7 Hz, 1H), 7.39 (t, J = 8.1 Hz, 2H), 7.09-7.03 (m, 2H), 6.67 (d, J = 5.1 Hz, 2H), 4.46 (s, 2H), 3.57 (s, 2H), 2.32 (dd, J = 8.4, 7.8 Hz, 2H), 1.98-1.93 (m, 2H). MS (m/z): 534.2 (M + H)⁺. |
| 49 | 13 | 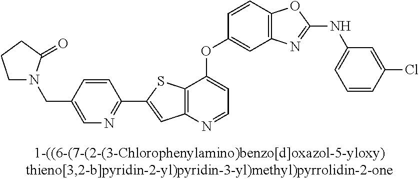 [SMILES: O=C1CCCN1Cc1ccc(-c2cc3nccc(Oc4ccc5oc(Nc6cccc(Cl)c6)nc5c4)c3s2)nc1] | ¹H-NMR (DMSO-d₆) δ (ppm): 8.51-8.50 (m, 2H), 8.33 (s, 1H), 8.24 (d, J = 8.1 Hz, 1H), 7.96 (s, 1H), 7.77 (d, J = 8.1 Hz, 1H), 7.65-7.60 (m, 2H), 7.50 (d, J = 2.1 Hz, 1H), 7.40 (t, J = 8.1 Hz, 1H), 7.11-7.08 (m, 2H), 6.66 (d, J = 5.4 Hz, 1H), 4.44 (s, 1H), 3.30-3.26 (m, 2H), 2.47 (t, J = 8.1 Hz, 2H), 1.99-1.91 (m, 2H). MS (m/z): 568.2 (M + H)⁺. |
| 50 | 14 | 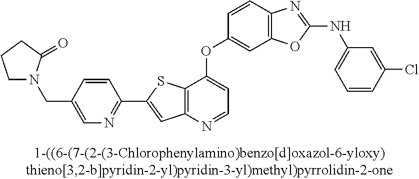 [SMILES: O=C1CCCN1Cc1ccc(-c2cc3nccc(Oc4ccc5nc(Nc6cccc(Cl)c6)oc5c4)c3s2)nc1] | ¹H-NMR (DMSO-d₆) δ (ppm): 8.53-8.51 (m, 2H), 8.34 (s, 1H), 8.26 (d, J = 8.1 Hz, 1H), 7.82- 7.79 (m, 3H), 7.67 (d, J = 2.1 Hz, 1H), 7.57 (d, J = 8.4 Hz, 1H), 7.46 (m, 2H), 7.22 (dd, J = 8.4, 2.1 Hz, 1H), 6.68 (d, J = 8.7, 2.4 Hz, 1H), 6.58 (d, J = 5.4 Hz, 1H), 4.46 (s, 2H), 3.30-3.28 (m, 2H), 2.31 (t, J = 8.1 Hz, 2H), 1.99-1.93 (m, 2H). MS (m/z): 568.3 (M + H)⁺. |
| 51 | 15 | 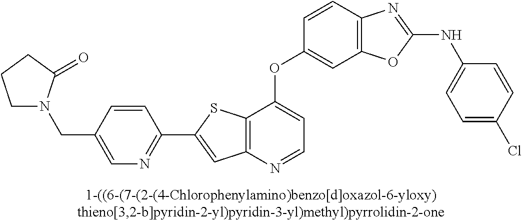 [SMILES: O=C1CCCN1Cc1ccc(-c2cc3nccc(Oc4ccc5nc(Nc6ccc(Cl)cc6)oc5c4)c3s2)nc1] | ¹H-NMR (CD₃OD) δ (ppm): 8.39 (d, J = 1.5 Hz, 1H), 8.30 (d, J = 5.7 Hz, 1H), 7.91 (d, J = 2.4 Hz, 2H), 7.79 (m, 1H), 7.67 (dd, J = 8.4, 2.1 Hz, 1H), 7.41 (dd, J = 8.4, 2.4 Hz, 2H), 7.27 (d, J = 2.1 Hz, 1H), 7.21 (t, J = 8.4 Hz, 1H), 7.04 (dd, J = 8.4, 2.1 Hz, 1H), 6.94 (m, 1H), 6.54 (d, J = 5.4 Hz, 1H), 4.42 (s, 2H), 3.28 (dd, J = 8.1, 6.9 Hz, 2H), 2.35 (t, J = 8.4 Hz, 2H), 1.98-1.91 (m, 2H). MS (m/z): 568.3 (M + H)⁺. |
| 52 | 16 | 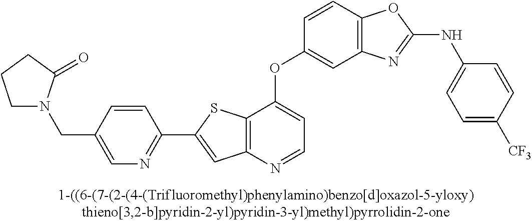 [SMILES: O=C1CCCN1Cc1ccc(-c2cc3nccc(Oc4ccc5oc(Nc6ccc(C(F)(F)F)cc6)nc5c4)c3s2)nc1] | ¹H-NMR (CD₃OD) δ (ppm): 8.55 (s, 1H), 8.49 (d, J = 5.4 Hz, 1H), 8.00 (s, 1H), 7.87-7.79 (m, 3H), 7.72-7.63 (m, 3H), 7.44-7.37 (m, 2H), 7.02 (d, J = 8.1 Hz, 1H), 6.59 (d, J = 5.1 Hz, 1H), 4.53 (s, 2H), 3.35 (t, J = 6.6 Hz, 2H), 2.48 (t, J = 7.8 Hz, 2H), 2.08-2.03 (m, 2H). MS (m/z): 602.0 (M + H)⁺. |
| 53 | 17 | 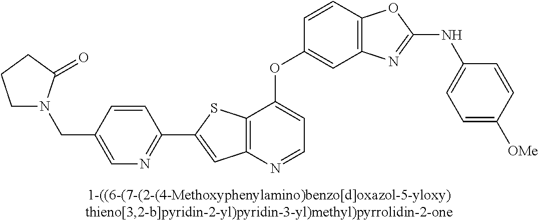 [SMILES: COc1ccc(Nc2nc3cc(Oc4ccnc5cc(-c6ccc(CN7CCCC7=O)cn6)sc45)ccc3o2)cc1] | ¹H-NMR (DMSO-d₆) δ (ppm): 10.6 (s, 1H), 8.52-8.50 (m, 2H), 8.34 (1H, s), 8.26 (d, J = 8.4 Hz, 1H), 7.79 (dd, J = 8.4, 1.8 Hz, 1H), 7.66 (d, J = 9.0 Hz, 2H), 7.58 (d, J = 8.7 Hz, 1H), 7.39 (d, J = 2.4 Hz, 1H), 7.03 (dd, J = 8.7, 2.4 Hz, 1H), 6.97 (d, J = 9.0 Hz, 2H), 6.66 (d, J = 5.4 Hz, 1H), 4.46 (s, 2H), 3.73 (s, 3H), 3.30 (t, J = 6.9 Hz, 2H), 2.31 (t, J = 8.1 Hz, 2H), 2.00-1.90 (m, 2H). MS (m/z): 564.1 (M + H)⁺. |
| 54 | 18 | 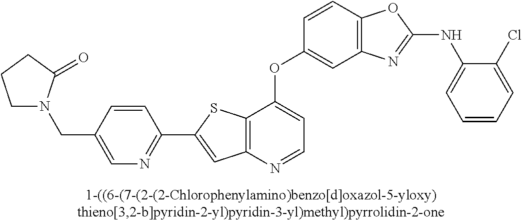 [SMILES: O=C1CCCN1Cc1ccc(-c2cc3nccc(Oc4ccc5oc(Nc6ccccc6Cl)nc5c4)c3s2)nc1] | ¹H-NMR (CDCl₃) δ (ppm): 8.61-8.55 (m, 2H), 8.50 (d, J = 5.7 Hz, 1H), 8.02 (s, 1H), 7.88 (d, J = 8.4 Hz, 1H), 7.75-7.71 (m, 1H) , 7.68-7.62 (brs, 1H), 7.47-7.38 (m, 4H), 7.10-7.03 (m, 2H), 6.60 (d, J = 5.4 Hz, 1H), 4.54 (s, 2H), 3.35 (t, J = 6.9 Hz, 2H), 2.49 (t, J = 8.1 Hz, 2H), 2.10-2.04 (m, 2H). MS (m/z): 568.3 (M + H)⁺. |
| 55 | 19 | 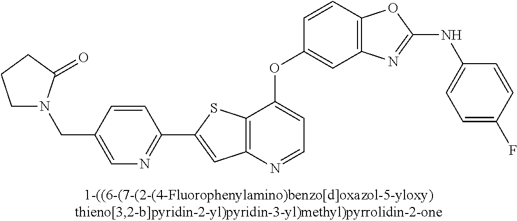 [SMILES: O=C1CCCN1Cc1ccc(-c2cc3nccc(Oc4ccc5oc(Nc6ccc(F)cc6)nc5c4)c3s2)nc1] | ¹H-NMR (CDCl₃) δ (ppm): 8.54 (d, J = 1.5 Hz, 1H), 8.48 (d, J = 5.4 Hz, 1H), 7.99 (s, 1H), 7.86- 7.84 (m, 2H), 7.73-7.69 (m, 1H), 7.64-7.59 (m, 2H), 7.397-.30 (m, 2H), 7.12-7.06 (m, 2H), 6.97 (dd, J = 8.4, 2.1 Hz, 1H), 6.58 (d, J = 5.4 Hz, 1H), 4.53 (s, 2H), 3.34 (t, J = 6.9 Hz, 2H), 2.48 (t, J = 8.1 Hz, 2H), 2.10-2.00 (m, 2H). MS (m/z): 552.2 (M + H)⁺. |
| 56 | 20 | 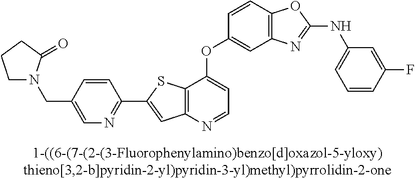 [SMILES: O=C1CCCN1Cc1ccc(-c2cc3nccc(Oc4ccc5oc(Nc6cccc(F)c6)nc5c4)c3s2)nc1] | ¹H-NMR (CDCl₃) δ (ppm): 11.07 (s, 1H), 8.53- 8.51 (m, 2H), 8.34 (s, 1H), 8.26 (d, J = 8.7 Hz, 2H), 7.81-7.75 (m, 2H), 7.65 (d, J = 8.7 Hz, 2H), 7.50-7.38 (m, 3H), 7.10 (dd, J = 8.7, 2.4 Hz, 1H), 6.91-6.85 (m, 1H), 6.67 (d, J = 5.7 Hz, 1H), 4.52 (s, 2H), 3.33 (t, J = 6.9 Hz, 2H), 2.47 (t, J = 8.1 Hz, 2H), 2.33 (s, 3H), 2.09-2.00 (m, 2H). MS (m/z): 552.1 (M + H)⁺. |
| 57 | 21 | 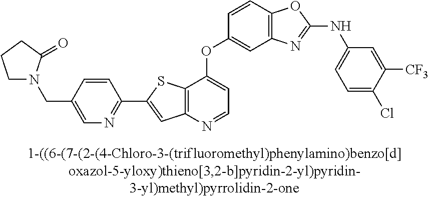 [SMILES: O=C1CCCN1Cc1ccc(-c2cc3nccc(Oc4ccc5oc(Nc6ccc(Cl)c(C(F)(F)F)c6)nc5c4)c3s2)nc1] | ¹H-NMR (CDCl₃) δ (ppm): 8.56 (s, 1H), 8.48 (d, J = 5.4 Hz, 1H), 8.03-7.99 (m, 2H), 7.87-7.83 (m, 1H), 7.74-7.68 (m, 1H), 7.50 (d, J = 8.7 Hz, 1H), 7.42-7.29 (m, 3H), 7.00 (dd, J = 8.7, 2.4 Hz, 1H), 6.58 (d, J = 5.7 Hz, 2H), 4.54 (s, 2H), 3.38- 3.33 (m, 2H), 2.49 (t, J = 8.1 Hz, 2H), 2.11- 2.03 (m, 2H). MS (m/z): 636.2 (M + H)⁺. |
| 58 | 22 | 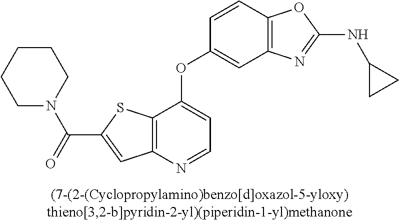 [SMILES: O=C(c1cc2nccc(Oc3ccc4oc(NC5CC5)nc4c3)c2s1)N1CCCCC1] | ¹H-NMR (CDCl₃) δ (ppm): 8.50 (d, J = 5.4 Hz, 1H), 7.63 (s, 1H), 7.28 (d, J = 8.7 Hz, 1H), 7.25 (d, J = 2.4 Hz, 1H), 6.88 (dd, J = 8.7, 2.4 Hz, 1H), 6.59 (d, J = 5.4 Hz, 1H), 5.42-5.39 (m, 1H), 3.68-3.73 (m, 4H), 2.91-2.86 (m, 1H), 1.77-1.70 (m, 6H), 0.97-0.90 (m, 2H), 0.78-0.72 (m, 2H). MS (m/z): 435.1 (M + H)⁺. |
| 59 | 23 | 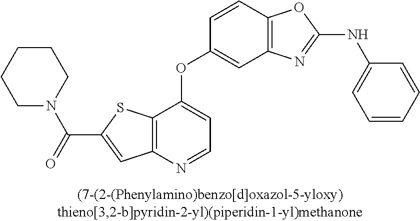 [SMILES: O=C(c1cc2nccc(Oc3ccc4oc(Nc5ccccc5)nc4c3)c2s1)N1CCCCC1] | ¹H-NMR (CDC1₃) δ (ppm): 8.50 (d, J = 5.4 Hz, 1H), 7.65-7.63 (m, 3H), 7.44-7.36 (m, 4H), 7.31 (d, J = 2.4 Hz, 1H), 7.16-7.11 (m, 1H), 6.96 (dd, J = 8.7, 2.4 Hz, 1H), 6.61 (d, J = 5.4 Hz, 1H), 3.72-3.68 (m, 4H), 1.77-1.60 (m, 6H). MS (m/z): 471.1 (M + H)⁺. |
| 60 | 24 | 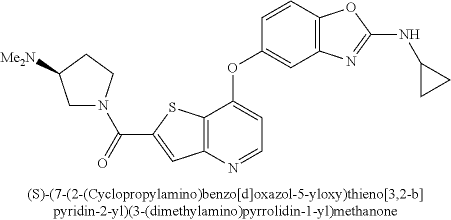 [SMILES: CN(C)[C@H]1CCN(C(=O)c2cc3nccc(Oc4ccc5oc(NC6CC6)nc5c4)c3s2)C1] | ¹H-NMR (CDCl₃) δ (ppm): 8.49 (d, J = 5.4 Hz, 1H), 7.85 (s, 1H), 7.30 (d, J = 8.4 Hz, 1H), 7.23 (d, J = 2.4 Hz, 1H), 6.87 (dd, J = 8.4, 2.4 Hz, 1H), 6.60 (d, J = 5.4 Hz, 1H), 5.80 (brs, 1H), 4.09-3.51 (m, 4H), 2.90-2.79 (m, 2H), 2.33 (s, 3H), 2.30 (s, 3H), 2.02-1.83 (m, 2H), 0.95-0.88 (m, 2H), 0.77-0.71 (m, 2H). MS (m/z): 464.2 (M + H)⁺. |
| 61 | 25 | 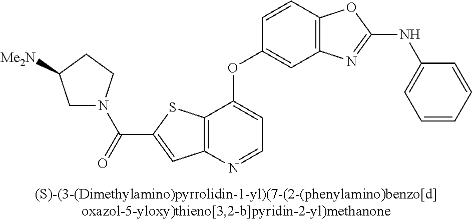 [SMILES: CN(C)[C@H]1CCN(C(=O)c2cc3nccc(Oc4ccc5oc(Nc6ccccc6)nc5c4)c3s2)C1] | ¹H-NMR (CDCl₃) δ (ppm): 8.51 (d, J = 5.4 Hz, 1H), 7.85 (s, 1H), 7.63 (d, J = 8.4 Hz, 2H), 7.44- 7.36 (m, 3H), 7.32 (d, J = 2.1 Hz, 1H), 7.16-7.11 (m, 2H), 6.97 (dd, J = 8.4, 2.4 Hz, 1H), 6.62 (d, J = 5.4 Hz, 1H), 4.08-3.51 (m, 4H), 2.90-2.79 (m, 1H), 2.33-2.20 (m, 7H), 2.03-1.88 (m, 1H). MS (m/z): 500.2 (M + H)⁺. |
| 62 | 26 | 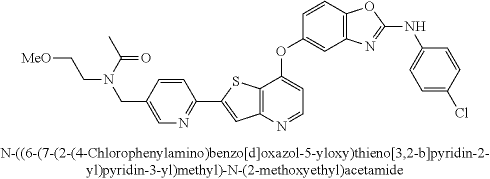 [SMILES: COCCN(Cc1ccc(-c2cc3nccc(Oc4ccc5oc(Nc6ccc(Cl)cc6)nc5c4)c3s2)nc1)C(C)=O] | ¹H-NMR (DMSO-d₆) δ (ppm): 8.52-8.49 (m, 2H), 8.35-8.21 (m, 2H), 7.79 (J = 8.4 Hz, 2H), 7.76 (brs, 1H), 7.63 (d, J = 9.0 Hz, 1H), 7.50- 7.43 (m, 3H), 7.08 (dd, J = 8.7, 2.1 Hz, 1H), 6.68-6.65 (m, 1H), 4.71 (s, 0.5H), 4.59 (s, 1.5H), 3.50-3.32 (m, 4H), 3.24 (s, 2.25H), 3.20 (s, 0.75H), 2.13 (s, 2.25H), 2.05 (s, 0.75H). MS (m/z): 600.3 (M + H)⁺. |
| 63 | 27 | 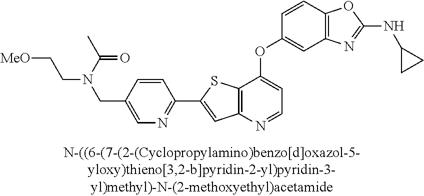 [SMILES: COCCN(Cc1ccc(-c2cc3nccc(Oc4ccc5oc(NC6CC6)nc5c4)c3s2)nc1)C(C)=O] | ¹H-NMR (CDCl₃) δ (ppm): 8.53 (s, 1H), 8.48 (d, J = 5.4 Hz, 0.36H), 8.46 (d, J= 5.4 Hz, 0.64H), 8.00 (s, 0.36H), 7.97 (s, 0.64H), 7.88 (d, J = 8.1 Hz, 0.36H), 7.83 (d, J = 8.1 Hz, 0.64H), 7.74 (dd, J = 8.1, 2.1 Hz, 0.64H), 7.63 (d, J = 8.1 Hz, 0.36H), 7.30 (d, J = 8.4 Hz, 1H), 7.24 (d, J = 2.7 Hz, 1H), 6.89 (dd, J = 8.4, 2.7 Hz, 1H), 6.55 (d = 5.4 Hz, 0.36H), 6.54 (d = 5.4 Hz, 0.64H), 5.31 (s, 1H), 4.74 (s, 0.72H), 4.70 (s, 1.28H), 3.61-3.47 (m, 4H), 3.32 (s, 1.92H), 3.30 (s, 1.08H), 2.93-2.85 (m, 1H), 2.22 (s, 1.92H), 2.17 (s, 1.08H), 0.96-0.90 (m, 2H), 0.77-0.72 (m, 2H). MS (m/z): 530.2 (M + H)⁺. |
| 64 | 28 | 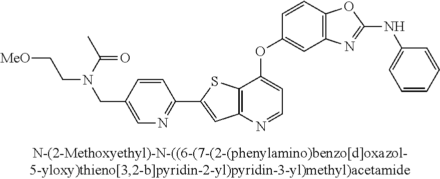 [SMILES: COCCN(Cc1ccc(-c2cc3nccc(Oc4ccc5oc(Nc6ccccc6)nc5c4)c3s2)nc1)C(C)=O] | ¹H-NMR (DMSO-d₆) δ (ppm): 10.8 (s, 1H), 8.53-8.50 (m, 3H), 8.35 (s, 0.25H), 8.32 (s, 0.75H), 8.28 (d, J = 8.1 Hz, 0.25H), 8.22 (d, J = 8.1 Hz, 0.75H), 7.78-7.75 (m, 3H), 7.62 (d, J = 8.4 Hz, 1H), 7.44 (d, J = 2.1 Hz, 1H), 7.39 (t, J = 7.8 Hz, 2H), 7.08-7.03 (m, 2H), 6.68-6.65 (m, 1H), 4.71 (s, 0.5H), 4.59 (s, 1.5H), 3.57 (s, 3H), 3.50-3.42 (m, 4H), 3.24 (s, 2.25H), 3.21 (s, 0.75H), 2.13 (s, 2.25H), 2.05 (s, 0.75H). MS (m/z): 566.2 (M + H)⁺. |
| 65 | 29 | 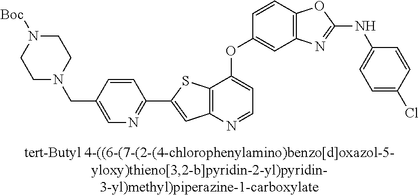 [SMILES: CC(C)(C)OC(=O)N1CCN(Cc2ccc(-c3cc4nccc(Oc5ccc6oc(Nc7ccc(Cl)cc7)nc6c5)c4s3)nc2)CC1] | ¹H-NMR (DMSO-d₆) δ (ppm): 10.95 (brs, 1H), 8.54 (d, J = 1.5 Hz, 1H), 8.50 (d, J = 5.7 Hz, 1H), 8.31 (s, 1H), 8.23 (d, J = 8.1 Hz, 1H), 7.85 (dd, J = 8.1, 2.1 Hz, 1H), 7.80 (s, 1H), 7.77 (d, J = 2.1 Hz, 1H), 7.62 (d, J = 8.4 Hz, 1H), 7.45- 7.42 (m, 3H), 7.07 (dd, J = 8.7, 2.4 Hz, 1H), 6.65 (d, J = 5.1 Hz, 1H), 3.56 (s, 2H), 3.38-3.26 (m, 4H), 2.36-2.34 (m, 4H), 1.38 (s, 9H). MS (m/z): 669.3 (M + H)⁺. |

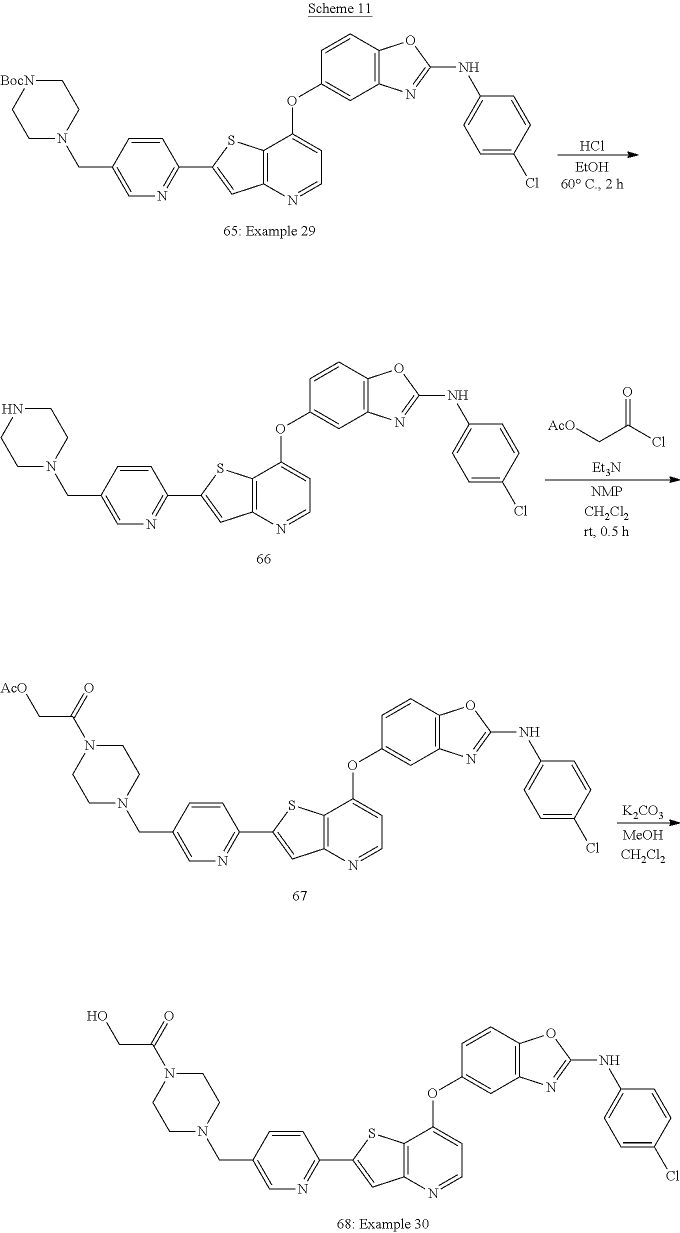

### Example 30

### 1-(4-((6-(7-(2-(4-Chlorophenylamino)benzo[d]oxazol-5-yloxy)thieno[3,2-b]pyridin-2-yl)pyridin-3-yl)methyl)piperazin-1-yl)-2-hydroxyethanone (68)

### Step 1: N-(4-Chlorophenyl)-5-(2-(5-(piperazin-1-ylmethyl)pyridin-2-yl)thieno[3,2-b]pyridin-7-yloxy)benzo[d]oxazol-2-amine (66)

To compound 65 (164 mg, 0.245 mmol) was added 1M HCl in EtOH and the reaction mixture was stirred for 2 h at 60° C. The reaction mixture was allowed to cool to ambient temperature, concentrated and treated with 1M aqueous NaOH solution (4.0 mL) to form a precipitate that was collected by filtration and dried to give title compound 66 (326 mg) of as a brown solid. The material was used in the next step with no additional purification and without characterization.

### Step 2: 2-(4-((6-(7-(2-(4-Chlorophenylamino)benzo[d]oxazol-5-yloxy)thieno[3,2-b]pyridin-2-yl)pyridin-3-yl)methyl)piperazin-1-D-2-oxoethyl acetate (67)

To a solution of the compound 66 (326 mg, <0.25 mmol) in a solvent mixture CH₂Cl₂/NMP (5+1 mL) was added Et₃N (81 mg, 0.80 mmol, 3.2 eq) and acetoxyacetyl chloride (55 mg, 0.40 mmol, 1.6 eq) at room temperature. The reaction mixture was stirred for 30 min and treated with aqueous NaOH solution (0.66M, 1.5 mL) to form a precipitate that was collected by filtration, absorbed on NH silica, applied onto a column and subjected to flash chromatigraphy (NH silica, eluent:AcOEt/Hexane 50/50-100/0), to afford title compound 67 (52 mg) as a colorless solid. The material was used in the next step without characterization.

### Step 3: 1-(4-((6-(7-(2-(4-Chlorophenylamino)benzo[d]oxazol-5-yloxy)thieno[3,2-b]pyridin-2-yl)pyridin-3-yl)methyl)piperazin-1-yl)-2-hydroxyethanone (68)

To the solution of the compound 67 (52 mg, 0.078 mmol) in a solvent mixture MeOH/CH₂Cl₂ (2 ml, each) was added K₂CO₃ (26 mg, 0.19 mmol) at room temperature. The reaction mixture was stirred for 1 h, absorbed on NH silica, applied onto a column and subjected to flash chromatography (NH silica, eluent: CH₂Cl₂/MeOH 99/1-92/8), to afford title compound 68 (44 mg, 29% yield over the three steps) as a colorless solid. ¹H-NMR (DMSO-d₆) δ: 8.55 (s, 1H), 8.50 (d, J=5.7 Hz, 1H), 8.31 (s, 1H), 8.24 (d, J=8.1 Hz, 1H), 7.86 (dd, J=8.1, 1.8 Hz, 1H), 7.78 (d, J=8.7 Hz, 2H), 7.62 (d, J=8.7 Hz, 1H), 7.45-7.42 (m, 3H), 7.07 (dd, J=5.4, 2.1 Hz, 1H), 6.66 (d, J=5.4 Hz, 1H), 4.53 (t, J=5.1 Hz, 1H), 4.06 (d, J=5.1 Hz, 2H), 3.58 (s, 2H), 3.53-3.30 (m, 4H), 2.46-2.38 (m, 4H). MS (m/z): 627.3 (M+H)⁺.

### Pharmaceutical Compositions

In some embodiments, the invention provides pharmaceutical compositions comprising a compound according to the invention and a pharmaceutically acceptable carrier, excipient, or diluent. Compositions of the invention may be formulated by any method well known in the art and may be prepared for administration by any route, including, without limitation, parenteral, oral, sublingual, transdermal, topical, intranasal, intratracheal, or intrarectal. In some embodiments, compositions of the invention are administered intravenously in a hospital setting. In some embodiments, administration may be by the oral route.

The characteristics of the carrier, excipient or diluent will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

The active compound is included in the pharmaceutically acceptable carrier, excipient or diluent in an amount sufficient to deliver to a patient a therapeutically effective amount without causing serious toxic effects in the patient treated. The effective dosage range of a pharmaceutically acceptable derivative can be calculated based on the weight of the parent compound to be delivered. If the derivative exhibits activity in itself, the effective dosage can be estimated as above using the weight of the derivative, or by other means known to those skilled in the art.

### Inhibition of VEGF Receptor Signaling

In some embodiments the invention provides a method of inhibiting VEGF receptor signaling in a cell, comprising contacting a cell in which inhibition of VEGF receptor signaling is desired with an inhibitor of VEGF receptor signaling according to the invention. Because compounds of the invention inhibit VEGF receptor signaling, they are useful research tools for in vitro study of the role of VEGF receptor signaling in biological processes.

In some embodiments, inhibiting VEGF receptor signaling causes an inhibition of cell proliferation of the contacted cells.

### Assay Examples

### Inhibition of VEGF Activity

The following protocol was used to assay the compounds of the invention.

### Assay Example 1

### In Vitro Receptor Tyrosine Kinase Assay (VEGF receptor KDR)

This test measures the ability of compounds to inhibit the enzymatic activity of recombinant human VEGF receptor enzymatic activity.

A 1.6-kb cDNA corresponding to the catalytic domain of VEGFR2 (KDR) (Genbank accession number AF035121 amino acid 806 to 1356) is cloned into the Pst I site of the pDEST20 Gateway vector (Invitrogen) for the production of a GST-tagged version of that enzyme. This construct is used to generate recombinant baculovirus using the Bac-to-Bac™ system according to the manufacturer's instructions (Invitrogen).

The GST-VEGFR2806-1356 protein is expressed in Sf9 cells (Spodoptera frugiperda) upon infection with recombinant baculovirus construct. Briefly, Sf9 cells grown in suspension and maintained in serum-free medium (Sf900 II supplemented with gentamycin) at a cell density of about 2×106 cells/ml are infected with the above-mentioned viruses at a multiplicity of infection (MOI) of 0.1 during 72 hours at 27° C. with agitation at 120 rpm on a rotary shaker. Infected cells are harvested by centrifugation at 398 g for 15 min. Cell pellets are frozen at −80° C. until purification is performed.

All steps described in cell extraction and purification are performed at 4° C. Frozen Sf9 cell pellets infected with the GST-VEGFR2806-1356 recombinant baculovirus are thawed and gently resuspended in Buffer A (PBS pH 7.3 supplemented with 1 μg/ml pepstatin, 2 μg/ml Aprotinin and leupeptin, 50 μg/ml PMSF, 50 μg/ml TLCK and 10 μM E64 and 0.5 mM DTT) using 3 ml of buffer per gram of cells. Suspension is Dounce homogenized and 1% Triton X-100 is added to the homogenate after which it is centrifuged at 22500 g, 30 min., 4° C. The supernatant (cell extract) is used as starting material for purification of GST-VEGFR2806-1356.

The supernatant is loaded onto a GST-agarose column (Sigma) equilibrated with PBS pH 7.3. Following a four column volume (CV) wash with PBS pH 7.3+1% Triton X-100 and 4 CV wash with buffer B (50 mM Tris pH 8.0, 20% glycerol and 100 mM NaCl), bound proteins are step eluted with 5 CV of buffer B supplemented with 5 mM DTT and 15 mM glutathion. GST-VEGFR2806-1356 enriched fractions from this chromatography step are pooled based on U.V. trace i.e. fractions with high O.D.280. Final GST-VEGFR2806-1356 protein preparations concentrations are about 0.7 mg/ml with purity approximating 70%. Purified GST-VEGFR2806-1356 protein stocks are aliquoted and frozen at −80° C. prior to use in enzymatic assay.

Inhibition of VEGFR/KDR is measured in a DELFIA™ assay (Perkin Elmer). The substrate poly(Glu4, Tyr) is immobilized onto black high-binding polystyrene 96-well plates. The coated plates are washed and stored at 4° C. During the assay, the enzyme is pre-incubated with inhibitor and Mg-ATP on ice in polypropylene 96-well plates for 4 minutes, and then transferred to the coated plates. The subsequent kinase reaction takes place at 30° C. for 10-30 minutes. ATP concentrations in the assay are 0.6 uM for VEGFR/KDR (2× the Km). Enzyme concentration is 5 nM. After incubation, the kinase reactions are quenched with EDTA and the plates are washed. Phosphorylated product is detected by incubation with Europium-labeled anti-phosphotyrosine MoAb. After washing the plates, bound MoAb is detected by time-resolved fluorescence in a Gemini SpectraMax reader (Molecular Devices). Compounds are evaluated over a range of concentrations, and IC₅₀ values (concentration of compounds giving 50% inhibition of enzymatic activity) are determined. The results are shown in Table 5.

TABLE 5

| Cmpd # | VEGFR_IC50_UM | P_ERK_IC50_UM |
|---|---|---|
| 47 | 1.558 | 0.12 |
| 63 | 1.581 | |
| 48 | | |
| 64 | 0.047 | |
| 60 | | |
| 58 | | |
| 46 | 0.005 | |
| 61 | 0.019 | |
| 59 | 0.24 | |
| 57 | 0.018 | |
| 18 | 0.023 | |
| 19 | 0.012 | |
| 21 | 0.008 | |
| 13 | 0.007 | |
| 15 | 0.007 | |
| 49 | 0.002 | |
| 50 | 0.434 | |
| 51 | 0.278 | |
| 62 | 0.004 | |
| 52 | 0.006 | |
| 53 | 0.003 | |
| 54 | 0.139 | |
| 55 | 0.014 | |
| 56 | 0.029 | |
| 16 | 0.016 | |
| 12 | 0.006 | |
| 14 | 0.005 | |
| 17 | 0.0413 | |
| 65 | 0.232 | |

### Assay Example 2

### VEGF-Dependent Erk Phosphorylation

Cells and Growth Factor:

HUVEC cells are purchased from Cambrex Bio Science Walkersville, Inc and cultured according to the vendor's instructions. The full-length coding sequence of VEGF₁₆₅ is cloned using the Gateway Cloning Technology (Invitrogen) for baculovirus expression Sf9 cells. VEGF₁₆₅ is purified from conditioned media using a NaCl gradient elution from a HiTrap heparin column (GE Healthcare Life Sciences) followed by an imidazole gradient elution from a HiTrap chelating column (GE Healthcare Life Sciences), then buffer stored in PBS supplemented with 0.1% BSA and filter sterilized

Cell Assays:

Cells are seeded at 8000 cells/well of a 96 wells plate and grown for 48 hours. Cells are then grown overnight in serum and growth factor-free medium and exposed for 1.5 h to compounds dilutions. Following a 15 min incubation in medium, VEGF₁₆₅ (150 ng/ml) cells are lysed in ice-cold lysis buffer (50 mM HEPES, pH 7.4, 150 mM NaCl, 1.5 mM MgCl₂, 1% Triton X-100, 10% glycerol) containing 1 mM 4-(2 aminoethyl)benzenesulfonyl fluoride hydrochloride, 200 μM sodium orthovanadate, 1 mM sodium fluoride, 10 μg/mL leupeptin, 10 μg/mL aprotinin, 1 μg/mL pepstatin and 50 μg/mL Na-p-tosyl-L-lysine chloromethyl ketone hydrochloride and processed as Western blots to detect anti-phospho ERK1/2 (T202/Y204)(Cell Signaling Technologies).

Western Blot Analysis:

lysates samples from single treatment wells are separated on 5-20% SDS-PAGE gels and immunobloting is performed using Immobilon polyvinylidene difluoride membranes (Amersham) according to the manufacturer's instructions. The blots are washed in Tris-buffered saline with 0.1% Tween 20 detergent (TBST) and probed for antibodies against phospho-Thr202/Tyr204-ERK (Cell signaling technologies. Chemiluminescence detection (Amersham, ECL plus) is performed according to the manufacturer's instructions using a Storm densitometer (GE Healthcare; 800 PMT, 100 nM resolution) for imaging and densitometry analysis. Values of over the range of dilution are used to prepare IC₅₀ curves using a 4-parameter fit model. These curves are calculated using GraFit 5.0 software.

### Assay Example 3

### In Vivo Solid Tumor Disease Model

This test measures the capacity of compounds to inhibit solid tumor growth.

Tumor xenografts are established in the flank of female athymic CD1 mice (Charles River Inc.), by subcutaneous injection of 1×106 U87, A431 or SKLMS cells/mouse. Once established, tumors are then serially passaged s.c. in nude mice hosts. Tumor fragments from these host animals are used in subsequent compound evaluation experiments. For compound evaluation experiments female nude mice weighing approximately 20 g are implanted s.c. by surgical implantation with tumor fragments of ˜30 mg from donor tumors. When the tumors are approximately 100 mm3 in size (˜7-10 days following implantation), the animals are randomized and separated into treatment and control groups. Each group contains 6-8 tumor-bearing mice, each of which is ear-tagged and followed individually throughout the experiment.

Mice are weighed and tumor measurements are taken by calipers three times weekly, starting on Day 1. These tumor measurements are converted to tumor volume by the well-known formula (L+W/4)3 4/3π. The experiment is terminated when the control tumors reach a size of approximately 1500 mm³. In this model, the change in mean tumor volume for a compound treated group/the change in mean tumor volume of the control group (non-treated or vehicle treated)×100 (AT/AC) is subtracted from 100 to give the percent tumor growth inhibition (% TGI) for each test compound. In addition to tumor volumes, body weight of animals is monitored twice weekly for up to 3 weeks

### Assay Example 4

### VEGF-Induced Retinal Vascular Permeability in Rabbits

### Materials and Methods

This test measures the capacity of compounds to inhibit VEGF-induced retinal vascular permeability. Vascular permeability is the cause of severe vision loss in patients suffering from age-related macular degeneration (AMD). Female Dutch rabbits (˜2 kg; Kitayama LABES CO., LTD, Nagano, Japan) are anesthetized with pentobarbital and topically with 0.4% oxybuprocaine hydrochloride. Test articles or vehicle are injected into vitreous cavity after the dilation of the pupils with 0.5% tropicamide eye drop. Recombinant human VEGF₁₆₅ (500 ng; Sigma-Aldrich Co., St Louis, Mo.) is injected intravitreously 48 hr prior to the measurement of vitreous fluorescein concentration. Rabbits are anesthetized with pentobarbital and sequentially injected sodium fluorescein (2 mg/kg) via the ear vein. Pupils are dilated with 0.5% tropicamide eye drop, and ocular fluorescein levels are measured using the FM-2 Fluorotron Master (Ocumetrics, Mountain View, Calif.) 30 min after fluorescein injection. The fluorescein concentrations in vitreous are obtained at data points that are 0.25 mm apart from posterior-end along an optical axis. Vitreous fluorescence concentration is considered fluorescein leakage from retinal vasculature. The average fluorescence peaks of the test article treated groups are compared with that of the vehicle-treated group.

1. A compound comprising Formula (I): 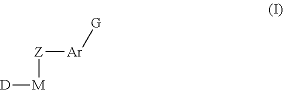
including N-oxides, hydrates, solvates, tautomers, pharmaceutically acceptable salts, prodrugs and complexes thereof, and racemic and scalemic mixtures, diastereomers and enantiomers thereof, wherein,
D is selected from the group consisting of an aromatic, heteroaromatic, cycloalkyl or heterocyclic ring system, C₁-C₆alkyl-heterocyclyl-C(O)—, C₁-C₆alkyl-heterocyclyl-C₁-C₆alkyl-N(R⁶)—C(O)—, (R⁶)(R⁶)N—C(O)—O-heterocyclyl-C(O)—, heterocyclyl-C(O)—, PivO-heterocyclyl-C(O)—, C₁-C₆alkyl-O—C(O)-heterocyclyl-C(O)—, C₁-C₆alkyl-C(O)—N(R⁶)-heterocyclyl-C(O)—, (C₁-C₆alkyl)(Box)N-heterocyclyl-C(O)—, HO-heterocyclyl-C(O)—, HO—C(O)-heterocyclyl-C(O)—, C₁-C₆alkyl-C(O)—O-heterocyclyl-C(O)—, (R⁶)(R⁶)N—C₁-C₆alky-N(R⁶)—C(O)-heterocyclyl-C(O)—, C₁-C₆alkyl-heterocyclyl-C(O)-heterocyclyl-C(O)— and (R⁶)(R⁶)N-heterocyclyl-C(O)—, wherein each of the aromatic, heteroaromatic, cycloalkyl and heterocyclic groups is optionally substituted with 1 or more independently selected R³⁸;
M is an optionally substituted fused heterocyclic moiety;
Z is selected from the group consisting of —O—, —S(O)₀₋₂— and —NR⁵—, wherein R⁵ is selected from the group consisting of H, optionally substituted C₁-C₅alkyl, an optionally substituted (C₁-C₅)acyl and C₁-C₆ alkyl-O—C(O), wherein C₁-C₆ alkyl is optionally substituted;
Ar is a group of the formulas C-1 or C-2, 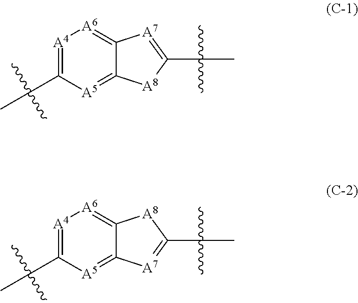
wherein A⁴, A⁵, A⁶ and A⁷ are independently selected from the group consisted of —CH— or N, and A⁸ is O, S(O)₀₋₂, CH₂, NH, NC₁₋₄-alkyl or NC₁₋₄-cycloalkyl;
G is a group B-L, wherein
B is selected from the group consisting of O, S(O)₀₋₂, CH₂, NH, NC₁₋₄-alkyl or NC₁₋₄-cycloalkyl;
L is independently selected from the group consisted of cycloalkyl, heterocyclyl, aryl or heteroaryl wherein the cycloalkyl, heterocyclyl, aryl or heteroaryl can be optionally substituted by 1-3 R²⁰;
wherein
R³⁸ is selected from the group consisting of C₂-C₆alkynyl-heterocyclyl, H(O)C— and C₁-C₆alkyl-C(O)—O—C₁-C₆alkyl-C(O)—, R³⁷O—C₁-C₆alkyl-C(O)-heterocyclyl-C₁-C₆alkyl-, R³⁷O—(CH₂)₁₋₆—N(A)-(CH₂)₁₋₄—, C₁-C₆alkyl-S(O)₂—(CH₂)₂—N(A)-CH₂—, R³⁷O—(CH₂)ⱼ—[(CH₂)ᵢO]ₓ—(CH₂)ᵢ₁—N(A)-(CH₂)ⱼ₁—, R³⁷O—C(O)—C₀-C₆alkyl-heterocyclyl-CH₂—, R³⁷O—(CH₂)ⱼ—[(CH₂)ᵢO]ₓ—(CH₂)ᵢ₁—N(R³⁹)—C(O)—, R³⁷—O—C(O)—C₁-C₆alkyl-heterocyclyl-C(O)—, HOOC—C₁-C₆alkyl-N(A)-CH₂—, (HOOC)(NR⁹R¹⁰)—C₁-C₆alkyl-N(A)-CH₂—, R³⁷O—C(O)—C₁-C₆alkyl-C(O)—, (R⁹)(R¹⁰)N—C₁-C₆alkyl-C(O)-heterocyclyl-CH₂—, cycloalkyl-N(R³⁹)—C(O)—O—C₁-C₆alkyl-, R³⁷—O—C₁-C₆alkyl-O—C₁-C₆alkyl-C(O)—, (R⁹)(R¹⁰)N—C(O)—C₁-C₆alkyl-heterocyclyl-CH₂—, (R⁹)(R¹⁰)N—C₁-C₆alkyl-C(O)—O—C₁-C₆alkyl-heterocyclyl-CH₂—, NC—C₁-C₆alkyl-heterocyclyl-CH₂—, F₃C—C₁-C₆alkyl-heterocyclyl-CH₂—, C₁-C₆alkyl-C(O)—O—C₁-C₆alkyl-C(O)-(5 to 10-membered heterocyclyl)-C₁-C₆alkyl-, (optionally substituted 8- to 10-membered fused heterocyclyl)-C₁-C₆alkyl-, F-heterocyclyl-C₁-C₆alkyl-, heteroaryl-C₁-C₆alkyl-heterocyclyl-C₁-C₆alkyl-, R³⁷—C₁-C₆alkyl-O—C₁-C₆alkyl-heterocyclyl-C₁-C₆alkyl-, R³⁷O—C(O)—C₁-C₆alkyl-O-heterocyclyl-C₁-C₆alkyl-, R³⁷O—C(O)—C₁-C₆alkyl-heterocyclyl-C₁-C₆alkyl-, heterocyclyl-C₁-C₆alkyl-O-aryl-N(R⁶)—C₁-C₆alkyl-, (heteroaryl substituted with one or more C₁-C₆alkyl)-N(R⁶)—C₁-C₆alkyl-, (C₁-C₆alkyl)₂N—C₁-C₆alkyl-aryl-N(R⁶)—C₁-C₆alkyl-, (C₁-C₆alkyl)₂N—C₁-C₆alkyl-C(O)-aryl-N(R⁶)—C₁-C₆alkyl-, heterocyclyl-C₁-C₆alkyl-O-aryl-N(R⁶)—C₁-C₆alkyl-, (R⁶)₂N-heterocyclyl-C₁-C₆alkyl-, C₁-C₆alkyl-C(O)—N(R⁶)-heterocyclyl-C₁-C₆alkyl-, C₁-C₆alkylC(O)—O—C₁-C₆alkyl-C(O)—N(R⁶)-heterocyclyl-C₁-C₆alkyl-, R³⁷O—C₁-C₆alkyl-C(O)—N(R⁶)-heterocyclyl-C₁-C₆alkyl-, heteroaryl-C₁-C₆alkyl-C(O)—N(R⁶)-heterocyclyl-C₁-C₆alkyl-, C₁-C₆alkyl-S(O)₂—N(R⁶)-heterocyclyl-C₁-C₆alkyl-, C₁-C₆alkyl-O—C(O)—N(R⁶)-heterocyclyl-C₁-C₆alkyl-, C₁-C₆alkyl-N(R⁶)—C(O)—N(R⁶)-heterocyclyl-C₁-C₆alkyl-, C₁-C₆alkyl-heterocyclyl-C(O)—N(R⁶)-heterocyclyl-C₁-C₆alkyl-, R³⁷O—C₁-C₆alkyl-N(R⁶)—C(O)—N(R⁶)-heterocyclyl-C₁-C₆alkyl-, (heterocyclyl optionally substituted with one or more C₁-C₆alkyl)-C₁-C₆alkyl-, (C₁-C₆alkyl)₂N—C₁-C₆alkyl-, C₁-C₆alkyl-heterocyclyl-C(O)—C₁-C₆alkyl-, heterocyclyl-C(O)—C₁-C₆alkyl-, C₁-C₆alkyl-O—C(O)—C₁-C₆alkyl-, C₁-C₆alkyl-O—C(O)—C₁-C₆alkyl-heteroaryl-N(R⁶)—C(O)—C₁-C₆alkyl-, (C₁-C₆alkyl)₂N-heterocyclyl-C(O)—C₁-C₆alkyl-, heteroaryl-C₁-C₆alkyl-N(R⁶)—C(O)—C₁-C₆alkyl-, (Boc)(H)N-heterocyclyl-C(O)—C₁-C₆alkyl-, C₁-C₆alkyl-O—C(O)-heterocyclyl-C(O)—C₁-C₆alkyl-, Boc-heterocyclyl-C(O)—C₁-C₆alkyl-, Ac—O—C₁-C₆alkyl-C(O)-heterocyclyl-C(O)—C₁-C₆alkyl-, R³⁷O—C₁-C₆alkyl-C(O)-heterocyclyl-C(O)—C₁-C₆alkyl-, (Boc)(H)N—C₁-C₆alkyl-C(O)-heterocyclyl-C(O)—C₁-C₆alkyl-, NH₂—C₁-C₆alkyl-C(O)-heterocyclyl-C(O)—C₁-C₆alkyl-, (C₁-C₆alkyl)(H)N—C(O)-heterocyclyl-C(O)—C₁-C₆alkyl-, NH₂-heterocyclyl-C(O)—C₁-C₆alkyl-, R³⁷O—C₁-C₆alkyl-O—C₁-C₆alkyl-heterocyclyl-C(O)—, C₁-C₆alkyl-O—C(O)—N(R⁶)-heterocyclyl-C(O)—, (R⁶)(R⁶)N-heterocyclyl-C(O)—, (R⁶)(R⁶)N-heterocyclyl-C₁-C₆alkyl-, heterocyclyl-O—C₁-C₆alkyl-, C₁-C₆alkyl-N(R⁶)—C(O)—N(R⁶)-heterocyclyl-C(O)—, (R⁶)(R⁶)N—C(O)-heterocyclyl-O—C₁-C₆alkyl-, C₂-C₆alkenyl-C(O)—N(R⁶)-heterocyclyl-C₁-C₆alkyl-, R³⁷O—C₁-C₆alkyl-C(O)-heterocyclyl-O—C₁-C₆alkyl-, R^{37a}—C₁-C₆alkyl-N(R⁶)-heterocylcyl-C₁-C₆alkyl-, R³⁷O—(CH₂)ⱼ—[(CH₂)₁O]ₓ—C₁-C₆alkyl-N(R⁶)-heterocyclyl-C₁-C₆alkyl-, halogen-C₁-C₆alkyl-heterocyclyl-C₁-C₆alkyl-, halogen-C₁-C₆alkyl-N(R⁶)-heterocyclyl-C₁-C₆alkyl-, R³⁷O—C(O)—C₁-C₆alkyl-N(R⁶)-heterocyclyl-C₁-C₆alkyl-, R³⁷—O—C(O)—C₁-C₆alkyl-N(R⁶)—C(O)—N(R⁶)-heterocyclyl-C₁-C₆alkyl-, (C₁-C₆alkyl)(H)N—C(O)-heterocyclyl-N[C₁-C₆alkyl-C(O)—OH]—C₁-C₆alkyl-, C₁-C₆alkyl-O—C(O)-heterocylcyl-C₁-C₆alkyl-, HO—C(O)-heterocyclyl-C₁-C₆alkyl-, C₁-C₆alkyl-heterocyclyl-C(O)-heterocyclyl-C₁-C₆alkyl-, R³⁷O—C₁-C₆alkyl-N(R⁶)—C(O)-heterocyclyl-C₁-C₆alkyl-, (R⁶)(R⁶)N—C₁-C₆alkyl-N(R⁶)—C(O)-heterocyclyl-C₁-C₆alkyl-, (C₁-C₆alkyl)(C₁-C₆alkyl)N-heterocyclyl-C₁-C₆alkyl-, R³⁷O—C₁-C₆alkyl-C(O)-[(C₁-C₆alkyl)(C₁-C₆alkyl)heterocyclyl]-C₁-C₆alkyl-, C₂-C₆alkenyl-C(O)-[(C₁-C₆alkyl)(C₁-C₆alkyl)heterocyclyl]-C₁-C₆alkyl-, R³⁷—O—C₁-C₆alkyl-[(C₁-C₆alkyl)(C₁-C₆alkyl)heterocyclyl]-C₁-C₆alkyl-, C₁-C₆alkyl-O—C₁-C₆alkyl-NR(⁶)—C₁-C₆alkyl-, C₁-C₆alkyl-O—C₁-C₆alkyl-N[C(O)—NH—C₁-C₆alkyl]-C₁-C₆alkyl-, C₁-C₆alkyl-O—C₁-C₆alkyl-N[C(O)—C₁-C₆alkyl]-C₁-C₆alkyl-, C₁-C₆alkyl-O—C₁-C₆alkyl-[C(O)—C₁-C₆alkyl-OH]-C₁-C₆alkyl-, R³⁷O—C(O)—C₁-C₆alkyl-C(O)-heterocyclyl-C₁-C₆alkyl-, R³⁷O—C(O)—C₁-C₆alkyl-heterocyclyl-C₁-C₆alkyl-, spiro-heterocyclyl-C₁-C₆alkyl-, R³⁷O—C₁-C₆alkyl-C(O)-spiro-heterocyclyl-C₁-C₆alkyl-, R³⁷O—C₁-C₆alkyl-C(O)-heterocyclyl-C₁-C₆alkyl-, C₁-C₆alkyl-heterocyclyl-C₁-C₆alkyl-, C₁-C₆alkyl-C(O)—O—C₁-C₆alkyl-C(O)-heterocyclyl-C₁-C₆alkyl-, heterocyclyl-C₁-C₆alkyl-C(O)-heterocyclyl-C₁-C₆alkyl-, (R⁶)(R⁶)N—C₁-C₆alkyl-C(O)-heterocyclyl-C₁-C₆alkyl-, heterocyclyl-C(O)-heterocyclyl-C₁-C₆alkyl-, (R⁶)(R⁶)N—C₂-C₆alkenyl-C(O)-heterocyclyl-C₁-C₆alkyl-, heterocyclyl-C₂-C₈alkenyl-C(O)-heterocyclyl-C₁-C₆alkyl-, (R⁶)(R⁶)N—C₁-C₆alkyl-N(R⁶)—C₁-C₆alkyl-C(O)-heterocyclyl-C₁-C₆alkyl-, heterocyclyl-C(O)—, (R⁶)(R⁶)N—C(O)-heterocyclyl-C₁-C₆alkyl-, R³⁷O—C(O)—C₁-C₆alkyl-N(R⁶)—C(O)-heterocyclyl-C₁-C₆alkyl-, C₂-C₆alkenyl-C(O)—O—C₁-C₆alkyl-N(R⁶)—C(O)-heterocyclyl-C₁-C₆alkyl-, (R⁶)(R⁶)N—C(O)-heterocyclyl-C(O)—, R³⁷O—C₁-C₆alkyl-N(R⁶)—C(O)-heterocyclyl-C₁-C₆alkyl-, R³⁷O—C₁-C₆alkyl-heterocyclyl-C₁-C₆alkyl-(heterocyclyl)-, R³⁷O—C(O)—C₁-C₆alkyl-heterocyclyl-C(O)—, R³⁷O—C₁-C₆alkyl-heterocyclyl-C(O)—, R³⁷O—C₁-C₆alkyl-C(O)-heterocyclyl-C(O)—, C₁-C₆alkyl-O—C(O)—N(R⁶)—C₁-C₆alkyl-C(O)—O—C₁-C₆alkyl-C(O)-heterocyclyl-C₁-C₆alkyl-, R³⁷O—(CH₂)ₙ[(CH₂)ᵢO]ₓ—C₁-C₆alkyl-N(R⁶)—C(O)-heterocyclyl-C₁-C₆alkyl-, HO-heterocyclyl-C₁-C₆alkyl-, R³⁷O-cycloalkyl-C(O)-heterocyclyl-C₁-C₆alkyl- and R³⁷O—(CH₂)ₙ[(CH₂)ᵢO]ₓ—C₁-C₆alkyl-C(O)—N(R⁶)-heterocyclyl-C₁-C₆alkyl;
A is selected from the group consisting of —C(O)—C₁-C₆alkyl-N(R³⁹)—C(O)—C₁-C₆alkyl-N(R⁹)(R¹⁰), —C(O)—N(R³⁹)—C₁-C₆alkyl, —C(═NR³⁷)—C₁-C₆alkyl, —C(O)—(CH₂)ₙ—S(O)₂—C₁-C₆alkyl, —C(O)—N(R³⁹)-cycloalkyl, —C(O)—N(R⁹)(R¹⁰), (R³⁷O)(R^{37a}0)P(O)O—C₁-C₆alkyl-C(O)—, —C(═NR³⁷)—H and —C₁-C₆alkyl-CF₃;
each R⁶ is independently H or C₁-C₆alkyl;
R³⁷ is selected from the group consisting of H, C₁-C₆alkyl and C₃-C₁₀cycloalkyl;
R^{37a} is selected from the group consisting of H, C₁-C₆alkyl and C₃-C₁₀cycloalkyl;
j is an integer ranging from 0 to 4, alternatively 0 to 2;
i is 2 or 3;
x is an integer ranging from 0 to 6, alternatively 2 or 3;
i1 is 2 or 3;
j1 is an integer ranging from 0 to 4, alternatively 1 or 2;
n is an integer ranging from 0 to 4;
R³⁹ is selected from the group consisting of H, —OH, C₁-C₆ alkyl, C₃-C₁₀ cycloalkyl, —(CH₂)ₙ₂(C₆-C₁₀ aryl), —(CH₂)ₙ₂(C₅-C₁₀ heteroaryl), —(CH₂)ₙ₂(5-10 membered heterocyclyl), —(CH₂)ₙ₂—O—(CH₂)ᵢ₂OR³⁷ and —(CH₂)ₙ₂OR³⁷, wherein the alkyl, aryl, heteroaryl and heterocyclyl moieties of the foregoing R³⁹ groups are optionally substituted;
R⁹ is selected from the group consisting of H, —OH, C₁-C₆ alkyl, C₃-C₁₀ cycloalkyl, —(CH₂)ₙ₃(C₆-C₁₀ aryl), —(CH₂)ₙ₃(C₅-C₁₀ heteroaryl), —(CH₂)ₙ₃(5-10 membered heterocyclyl), —(CH₂)ₙ₃O(CH₂)ᵢ₃OR³⁷ and —(CH₂)ₙ₃OR³⁷, wherein the alkyl, aryl, heteroaryl and heterocyclyl moieties of the foregoing R⁹ groups are optionally substituted;
R¹⁰ is selected from the group consisting of H, —OH, C₁-C₆ alkyl, C₃-C₁₀ cycloalkyl, —(CH₂)ₙ₄(C₆-C₁₀ aryl), —(CH₂)ₙ₄(C₅-C₁₀ heteroaryl), —(CH₂)ₙ₄(5-10 membered heterocyclyl), —(CH₂)ₙ₄O(CH₂)ᵢ₄OR³⁷ and —(CH₂)ₙ₄OR³⁷, wherein the alkyl, aryl, heteroaryl and heterocyclyl moieties of the foregoing R¹⁰ groups are optionally substituted;
R²⁰ is selected from the group consisting of —H, halogen, trihalomethyl, —CN, —NO₂, —NH₂, —OR³, C₃₋₆-cycloalkyl, C₁₋₆-alkoxy, C₃₋₆-cycloalkoxy, CF₃, CCl₃, —OCF₃, —NR³R⁴, —S(O)₀₋₂R³, —S(O)₂NR³R³, —C(O)OR³, —C(O)NR³R³, —N(R³)SO₂R³, —N(R³)C(O)R³, —N(R³)C(O)OR³, —C(O)R³, —C(O)SR³, C₁-C₄ alkoxy, C₁-C₄ alkylthio, —O(CH₂)ₙ₆aryl, —O(CH₂)ₙ₆heteroaryl, —(CH₂)ₙ₆(aryl), —(CH₂)ₙ₆(heteroaryl), C₁-C₆ alkyl, C₂-C₆ alkenyl, C₂-C₆ alkynyl, —CH₂(CH₂)₀₋₄-T², an optionally substituted C₁₋₄ alkylcarbonyl, C₁₋₄ alkoxy, an amino optionally substituted by C₁₋₄ alkyl optionally substituted by C₁₋₄ alkoxy, —(CH₂)ₙ₆P(═O)(C₁-C₆alkyl)₂, a saturated or unsaturated three- to seven-membered cycloalkyl or heterocyclic group, —SiMe₃ and —SbF₅;
n2 is an integer ranging from 0 to 6;
i2 is an integer ranging from 2 to 6;
n3 is an integer ranging from 0 to 6;
i3 is an integer ranging from 2 to 6;
n4 is an integer ranging from 0 to 6; and
i4 is an integer ranging from 2 to 6
each R³ is independently selected from the group consisting of —H and R⁴;
R⁴ is selected from the group consisting of a (C₁-C₆)alkyl, an aryl, a lower arylalkyl, a heterocyclyl and a lower heterocyclyl-alkyl, each of which is optionally substituted, or
R³ and R⁴, taken together with a common nitrogen to which they are attached, form an optionally substituted five- to seven-membered heterocyclyl, the optionally substituted five- to seven-membered heterocyclyl optionally containing at least one additional annular heteroatom selected from the group consisting of N, O, S and P;
T² is selected from the group consisting of —OH, —OMe, —OEt, —NH₂, —NHMe, —NMe₂, —NHEt and —NEt₂, and wherein the aryl, heteroaryl, C₁-C₆ alkyl, C₂-C₆ alkenyl, and C₂-C₆ alkynyl are optionally substituted; and
n6 is an integer ranging from 0 to 6.

2. The compound according to claim 1, wherein the compound is

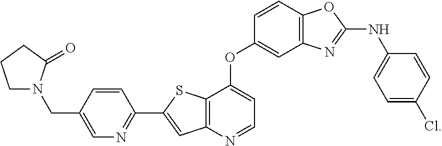 [SMILES: O=C1CCCN1Cc1ccc(-c2cc3nccc(Oc4ccc5oc(Nc6ccc(Cl)cc6)nc5c4)c3s2)nc1]

3. The compound according to claim 1, wherein the compound is

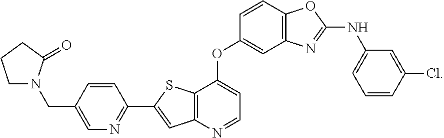 [SMILES: O=C1CCCN1Cc1ccc(-c2cc3nccc(Oc4ccc5oc(Nc6cccc(Cl)c6)nc5c4)c3s2)nc1]

4. The compound according to claim 1, wherein the compound is

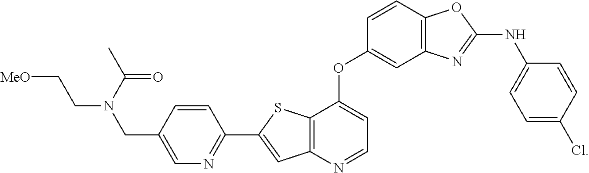 [SMILES: COCCN(Cc1ccc(-c2cc3nccc(Oc4ccc5oc(Nc6ccc(Cl)cc6)nc5c4)c3s2)nc1)C(C)=O]

5. The compound according to claim 1, wherein the compound is

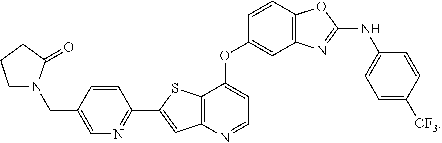 [SMILES: O=C1CCCN1Cc1ccc(-c2cc3nccc(Oc4ccc5oc(Nc6ccc(C(F)(F)F)cc6)nc5c4)c3s2)nc1]

6. The compound according to claim 1, wherein the compound is

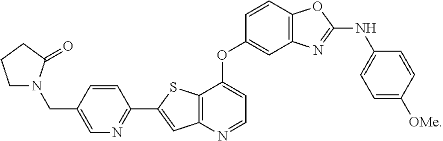 [SMILES: COc1ccc(Nc2nc3cc(Oc4ccnc5cc(-c6ccc(CN7CCCC7=O)cn6)sc45)ccc3o2)cc1]

7. The compound according to claim 1, wherein the compound is

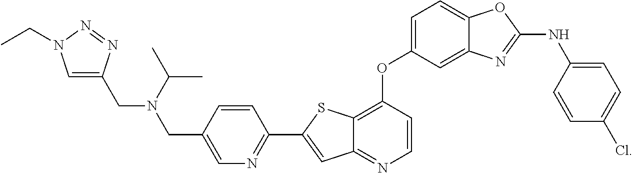 [SMILES: CCn1cc(CN(Cc2ccc(-c3cc4nccc(Oc5ccc6oc(Nc7ccc(Cl)cc7)nc6c5)c4s3)nc2)C(C)C)nn1]

8. The compound according to claim 1, wherein the compound is

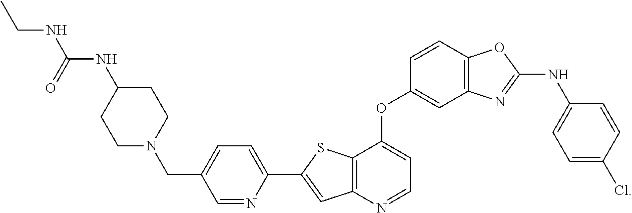 [SMILES: CCNC(=O)NC1CCN(Cc2ccc(-c3cc4nccc(Oc5ccc6oc(Nc7ccc(Cl)cc7)nc6c5)c4s3)nc2)CC1]

9. The compound according to claim 1, wherein the compound is

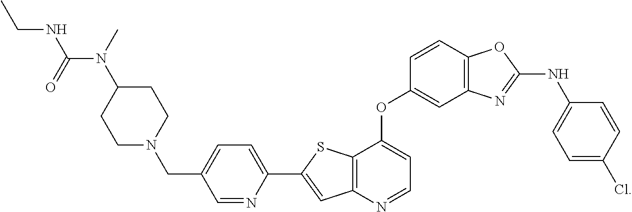 [SMILES: CCNC(=O)N(C)C1CCN(Cc2ccc(-c3cc4nccc(Oc5ccc6oc(Nc7ccc(Cl)cc7)nc6c5)c4s3)nc2)CC1]

10. The compound according to claim 1, wherein the compound is

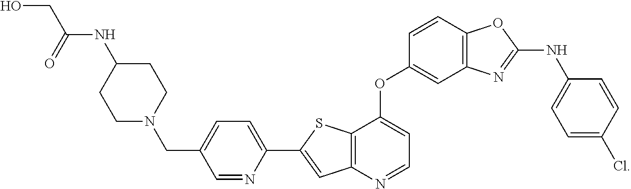 [SMILES: O=C(CO)NC1CCN(Cc2ccc(-c3cc4nccc(Oc5ccc6oc(Nc7ccc(Cl)cc7)nc6c5)c4s3)nc2)CC1]

11. The compound according to claim 1, wherein the compound is

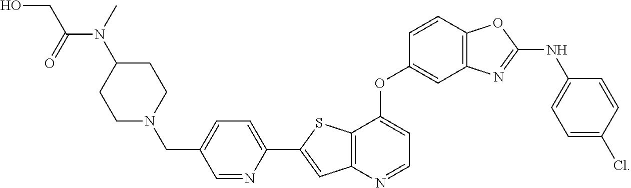 [SMILES: CN(C(=O)CO)C1CCN(Cc2ccc(-c3cc4nccc(Oc5ccc6oc(Nc7ccc(Cl)cc7)nc6c5)c4s3)nc2)CC1]

12. A composition comprising a compound according to claim 1 and a pharmaceutically acceptable carrier.

13. A method of treating an opthalmic disease, condition or disorder, the method comprising administering to a patient in need thereof a therapeutically effective amount of a compound according to claim 1 or a composition thereof, wherein the ophthalmic disease, disorder or condition is selected from the group consisting of (a) a disease, disorder or condition caused by choroidal angiogenesis, (b) diabetic retinopathy and (c) retinal oedema.

14. The method according to claim 13, wherein the ophthalmic disease, disorder or condition is age-related macular degeneration.
